(19) United States
(12) Patent Application Publication
(10) Pub. No.: US 2016/0130665 A1
(43) Pub. Date: May 12, 2016

(54) PROGNOSTIC METHODS AND SYSTEMS FOR CHRONIC LYMPHOCYTIC LEUKEMIA

(71) Applicant: Cancer Genetics, Inc., Rutherford, NJ (US)

(72) Inventors: Raju S.K. Chaganti, Hillsdale, NJ (US); Jane Houldsworth, Franklin Lakes, NJ (US)

(21) Appl. No.: 14/938,230

(22) Filed: Nov. 11, 2015

Related U.S. Application Data
(60) Provisional application No. 62/078,151, filed on Nov. 11, 2014.

(51) Int. Cl. C12Q 1/68

(52) U.S. Cl. CPC C12Q 1/6886; C12Q 2600/118; C12Q 2600/158; C12Q 2600/156

(57) ABSTRACT

The present invention provides systems useful for risk stratification of chronic lymphocytic leukemia (CLL) patients. The systems can include a microarray and a decision tree having steps for stratification of one or more CLL patients into prognostic groups. The invention further provides methods for risk stratification of CLL patients. The methods can include detecting the presence of alterations, such as copy number alterations, in sample genetic material from each of one or more CLL patients and then stratifying the one or more CLL patients into prognostic groups.

### CROSS-REFERENCE TO RELATED APPLICATIONS

This application claims the benefit of U.S. Patent Application No. 62/078,151, filed Nov. 11, 2014, which is herein incorporated by reference in its entirety for all purposes.

### FIELD OF THE INVENTION

The present invention provides a tool useful in the prognosis of chronic lymphocytic leukemia (CLL). The tool can utilize a specific array-comparative genomic hybridization genome scanning technique to determine the prognosis of a CLL patient. The invention thus also provides methods for the prognosis of such malignancies, preferentially with minimal invasiveness.

### REFERENCE TO A SEQUENCE LISTING

A sequence listing is incorporated herein by reference in its entirety. The listing, in ASCII format, was created on Nov. 11, 2015, is named 471798SEQLIST.txt, and is 2.43 kilobytes in size.

### BACKGROUND OF THE INVENTION

Chronic lymphocytic leukemia (CLL) is a type of mature B-cell neoplasm that occurs almost exclusively in adults with a median age at diagnosis of 65 to 68 years. It comprises approximately 10% of all adult hematologic malignancies, but 40% of leukemias in individuals over 65 years of age. In the U.S., approximately 15,000 new cases are diagnosed each year (Jemal et al., CA Cancer J. Clin. 59:225-249 (2009)). At the present time, CLL is often detected in asymptomatic patients with an elevated lymphocyte count in a routine full blood count (Hallek et al., Blood 111:5446-5456 (2008)). Definitive diagnosis is based on a lymphocytosis and characteristic lymphocyte morphology and immunophenotype (Hallek et al., Blood 111:5446-5456 (2008)). In this disease where some patients have aggressive disease requiring immunochemotherapy (fludarabine, cyclophosphamide, rituximab) and where others will survive for decades without therapy, there have been reports of the development of a prognostic index based on both clinical and laboratory features (Shanafelt et al., Cancer 115:363-372 (2009); Wierda et al., Blood 109:4679-4685 (2007)). With morphologic examination, diagnosis is also based on flow cytometry (kappa/lambda to assess clonality), and the distinguishing immunophenotype is CD5+, CD23+, FMC-7−, and CD20 dim. Fluorescence in situ hybridization (FISH) is recommended for the detection of 11q-, 13q-, +12, and 17p- which have prognostic value, and of t(11;14)(q13;q32) to distinguish CLL from mantle cell lymphoma (MCL) (Zenz et al., Best Pract. Res. Clin. Haematol. 20:439-453 (2007)). Mutation status of the variable region of IGH also has prognostic value where unmutated (<2% compared with germline) is associated with aggressive disease (Hamblin, Best Pract. Res. Clin. Haematol. 20:455-468 (2007)). CD38 and ZAP70 expression, as assessed by flow cytometry, are considered surrogates for IGH mutation status.

The clinical course of patients with CLL is highly variable, underscoring the importance of risk stratification to guide clinical management (Chiorazzi et al., N. Engl. J. Med. 352:804-815 (2005)). When therapeutic intervention is considered as the disease progresses, risk stratification is recommended to include assessment of overall fitness, comorbid conditions, and a few biomarkers including sequence analysis of the clonally rearranged IGH locus (Damle et al., Blood 94:1840-1847 (1999); Hamblin et al., Blood 94:1848-1854 (1999); NCCN, Non-Hodgkin's Lymphomas, NCCN Clinical Practice Guidelines in Oncology 2011, Version 4.2011). Also assessed is the presence of somatic genomic abnormalities by FISH including loss of 13q14, the TP53 (17p13) and ATM (11q22-q23) loci, and trisomy 12 (Shanafelt et al., J. Clin. Oncol. 24:4634-4641 (2006)). Currently, this probe combination dichotomizes patients into those carrying del(17p) or del(11q) (poor prognosis) and those who do not. This has reduced prognostic value compared with the original hierarchical model, which also permitted discrimination of patients with a favorable outcome but failed to classify all specimens (Dohner et al., N. Engl. J. Med. 343:1910-1916 (2000)).

Array-based comparative genomic hybridization (aCGH) and massively parallel-sequencing technologies have provided an opportunity for more comprehensive evaluations of the CLL genome, identifying gain, loss, and other mutational events that potentially have clinicopathologic relevance.

### SUMMARY OF THE INVENTION

The present invention provides for the assessment of genomic alterations in the prognosis of chronic lymphocytic leukemia (CLL). In particular, the invention provides the ability to use genome scanning technology, such as array comparative genomic hybridization (array-CGH or aCGH), as a clinical tool for the prognosis of CLL and for risk stratification of CLL patients. The invention provides various techniques, platforms, specimen cohort sizes, and modalities that can be useful to stratify one or more CLL patients into prognostic groups.

In one aspect, the invention provides a system for risk stratification of one or more CLL patients. In certain embodiments, a system according to the invention comprises a microarray and a decision tree. In certain embodiments, the microarray comprises a substrate with a plurality of distinct genomic regions arrayed thereon. Preferably, each of the distinct genomic regions individually is capable of hybridizing to material present in sample genetic material from the one or more CLL patients. Moreover, the genomic regions represented on the microarray can be regions wherein an alteration therein is correlated to one or more CLL prognostic groups. In certain embodiments, the decision tree comprises steps for stratification of one or more CLL patients into the following groups: (i) poor prognosis: the CLL patients whose sample genetic material comprises at least one of gain of 2p, gain of 3q, gain of 8q, gain of 17q, loss of 7q, loss of 8p, loss of 11q, loss of 17p, and loss of 18p; (ii) good prognosis: the CLL patients whose sample genetic material comprises loss of 13q14 without any of the copy number alterations listed in step (i) and without any of gain of 1p, gain of 7p, gain of 12, gain of 18p, gain of 18q, gain of 19, loss of 4p, loss of 5p, loss of 6q, and loss of 7p; and (iii) intermediate prognosis: all other CLL patients. In certain embodiments, the steps for stratification occur in the following order: step (i) occurs first, step (ii) occurs second, and step (iii) occurs third. In certain embodiments, the above gains or losses are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

In another aspect, the invention provides methods for risk stratification of one or more CLL patients. In certain embodiments, a method according to the invention comprise the following steps: (a) detecting the presence of copy number alterations in sample genetic material from each of said one or more CLL patients; and (b) stratifying each of said one or more CLL patients into one of the following groups: (i) poor prognosis: the CLL patients whose sample genetic material comprises at least one of gain of 2p, gain of 3q, gain of 8q, gain of 17q, loss of 7q, loss of 8p, loss of 11q, loss of 17p, and loss of 18p; (ii) good prognosis: the CLL patients whose sample genetic material comprises loss of 13q14 without any of the copy number alterations listed in step (b)(i) and without any of gain of 1p, gain of 7p, gain of 12, gain of 18p, gain of 18q, gain of 19, loss of 4p, loss of 5p, loss of 6q, and loss of 7p; and (iii) intermediate prognosis: all other CLL patients. In certain embodiments, the steps for stratification within step (b) occur in the following order: step (b)(i) occurs first, step (b)(ii) occurs second, and step (b)(iii) occurs third. In certain embodiments, the above gains or losses are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

### BRIEF DESCRIPTION OF THE DRAWINGS

These and other features, aspects and advantages of the present invention will become better understood with regard to the following description and accompanying drawings wherein:

FIG. 1A-D show the genomic landscape of the CLL genome for datasets 1 and 2 and classify treatment-naïve CLL specimens into one of three prognostic groups according to genomic imbalance as assessed by targeted aCGH. FIG. 1A shows the genomic landscape and prognostic groups for the CLL specimens from dataset 1 (DS1). FIG. 1B shows the genomic landscape for CLL specimens from dataset 2 (DS2) classified as poor outcome. FIG. 1C shows the genomic landscape for CLL specimens from dataset 2 classified as intermediate outcome. FIG. 1D shows the genomic landscape for CLL specimens from dataset 2 classified as good outcome. All specimens in datasets 1 and 2 were obtained from CLL patients with informed consent during routine care at the North Shore-LIJ Health System.

FIG. 2A-B show Kaplan-Meier plots for treatment-naïve specimens from datasets 1 and 2 that have been classified in a hierarchical manner into one of three groups (poor, intermediate, good) based on 20 copy number alterations (CNAs). FIG. 2A shows the time to first treatment (TTFT) clinical endpoint. FIG. 2B shows the overall survival (OS) clinical endpoint.

FIG. 3A-B show classifications of 13q14 deletions into type I or type II based on exclusion or inclusion of RB1 or a part thereof as well as losses of genes involved in 13q13 deletions. FIG. 3A shows classifications for specimens in dataset 1. FIG. 3B shows classifications for specimens in dataset 2.

FIG. 3C-F show an assessment of the clinical relevance of type I and type II deletions in treatment-naïve specimens of datasets 1 and 2 in all specimens with 13q14 deletions (All) and in those with 13q14 deletions as a sole abnormality (Sole). FIG. 3C shows the TTFT clinical endpoint for all specimens with 13q14 deletions. FIG. 3D shows the TTFT clinical endpoint for specimens with 13q14 deletions as a sole abnormality. FIG. 3E shows the OS clinical endpoint for all specimens with 13q14 deletions. FIG. 3F shows the OS clinical endpoint for specimens with 13q14 deletions as a sole abnormality.

FIG. 4A-T show Kaplan-Meier plots for various CNAs. The Kaplan-Meier plots using the TTFT clinical endpoint include plots for the following CNAs: 2p gain (FIG. 4A); 3q gain (FIG. 4B); 8q gain (FIG. 4C); 17q gain (FIG. 4D); 7q loss (FIG. 4E); 8p loss (FIG. 4F); 11q loss (FIG. 4G); 13q loss (FIG. 4H); 17p loss (FIG. 4I); and 18p loss (FIG. 4J). The Kaplan-Meier plots using the OS clinical endpoints include plots for the following CNAs: 2p gain (FIG. 4K); 3q gain (FIG. 4L); 8q gain (FIG. 4M); 17q gain (FIG. 4N); 7q loss (FIG. 4O); 8p loss (FIG. 4P); 11q loss (FIG. 4Q); 13q loss (FIG. 4R); 17p loss (FIG. 4S); and 18p loss (FIG. 4T).

FIG. 5A shows Kaplan-Meier plots with TTFT as the clinical endpoint for 13q loss as a sole abnormality (Sole) and 13q loss combined with other abnormalities (Not Sole).

FIG. 5B shows Kaplan-Meier plots with TTFT as the clinical endpoint for patients exhibiting only one or more of the following CNAs (gain: 1p, 7p, 12, 18p, 18q, 19, loss: 4p, 5p, 6q, 7p) (Other abs*) or exhibiting none of the following CNAs (gain: 2p, 3q, 8q, 17q; loss: 7q, 8p, 11q, 17p, 18p; gain: 1p, 7p, 12, 18p, 18q, 19; loss: 4p, 5p, 6q, 7p; loss: 13q14) (No abs*).

FIG. 5C shows Kaplan-Meier plots with OS as the clinical endpoint for 13q loss as a sole abnormality (Sole) and 13q loss combined with other abnormalities (Not Sole).

FIG. 5D shows Kaplan-Meier plots with OS as the clinical endpoint for patients exhibiting only one or more of the following CNAs (gain: 1p, 7p, 12, 18p, 18q, 19, loss: 4p, 5p, 6q, 7p) (Other abs*) or exhibiting none of the following CNAs (gain: 2p, 3q, 8q, 17q; loss: 7q, 8p, 11q, 17p, 18p; gain: 1p, 7p, 12, 18p, 18q, 19; loss: 4p, 5p, 6q, 7p; loss: 13q14) (No abs*).

FIG. 6A-F show Kaplan-Meier plots for IGHV mutational status (mutated=Mut; unmutated=Unmut). FIG. 6A shows a plot for the good prognosis group using TTFT as the clinical endpoint. FIG. 6B shows a plot for the intermediate prognosis group using TTFT as the clinical end point. FIG. 6C shows a plot for the poor prognosis group using TTFT as the clinical endpoint. FIG. 6D shows a plot for the good prognosis group using OS as the clinical endpoint. FIG. 6E shows a plot for the intermediate prognosis group using OS as the clinical end point. FIG. 6F shows a plot for the poor prognosis group using OS as the clinical endpoint.

FIG. 7A-D show Kaplan-Meier plots using the CLL prognostic classification scheme disclosed herein (good, intermediate, and poor) on two previously untreated CLL datasets: samples from Dana Farber Cancer Institute (DFCI) and samples from Hackensack University Medical Center (HUMC). FIG. 7A shows the TTFT clinical endpoint for the samples from DFCI. FIG. 7B shows the TTFT clinical endpoint for the samples from HUMC. FIG. 7C shows the OS clinical endpoint for the samples from DFCI. FIG. 7D shows the OS clinical endpoint for the samples from HUMC.

FIG. 8 shows a decision tree based on the CLL prognostic classification scheme disclosed herein.

### DETAILED DESCRIPTION OF THE INVENTION

The invention now will be described more fully hereinafter through reference to various embodiments. These embodiments are provided so that this disclosure will be thorough and complete, and will fully convey the scope of the invention to those skilled in the art. Indeed, the invention can be embodied in many different forms and should not be construed as limited to the embodiments set forth herein; rather, these embodiments are provided so that this disclosure will satisfy applicable legal requirements. As used in the specification, and in the appended claims, the singular forms “a,” “an,” and “the” include plural referents unless the context clearly dictates otherwise.

Various technical and scientific terms are used in the present disclosure, and the meaning of said terms is understood to be as expressly defined herein or as otherwise ascertainable from the context of the present disclosure. To the extent such terms are not expressly or inherently defined herein, the meaning of such terms is understood to be the same as commonly understood by one of ordinary skill in the art to which this invention belongs.

As used herein, the term “genomic region” is intended to mean a portion of nucleic acid polymer that is contained within the genome complement of any member of the animal kingdom that may be inflicted with CLL, preferably a mammal, and even more preferably a human. The term can relate to a specific length of DNA. The term can also be used in relation to specific oligonucleotides. Location of the nucleic acid polymer within the genome can be defined with respect to either the chromosomal band in the genome or one or more specific nucleotide positions in the genome.

As used herein, the term “chronic lymphocytic leukemia,” also referred to as “CLL,” is a cancer of the blood and bone marrow that affects B lymphocytes or B cells. CLL causes an accumulation in cancer cells (i.e., B cells), which spread through the bone marrow and blood. CLL can also affect lymph nodes and other organs.

As used herein, the term “CLL patient” is intended to mean any subject for whom a CLL prognosis is desired, including, for example, subjects who have CLL (e.g., treatment-naïve patients) and subjects who are suspected of having CLL. A “subject” can be any member of the animal kingdom that may be inflicted with CLL, preferably a mammal, and even more preferably a human.

As used herein, the term “treatment-naïve patient” is intended to mean any CLL patient who has never been treated for CLL with any form of CLL therapy. Such CLL therapies include, but are not limited to, FDA-approved CLL therapies and off-label CLL therapies that are generally accepted by physicians.

As used herein, the terms “biopsy” and “biopsy specimen” are intended to mean a biological sample of tissue, cells, or liquid taken from the body of a subject.

As used herein, the term “genetic material” is intended to mean materials comprising or formed predominantly of nucleic acids. The term specifically is intended to encompass, deoxyribonucleic acids (DNA) or fragments thereof and ribonucleic acids (RNA) or fragments thereof. The term can also be used in reference to genes, chromosomes, and/or oligonucleotides and can encompass any portion of the nuclear genome and/or the mitochondrial genome of a subject. Preferably, genetic material is DNA. More preferably, genetic material is chromosomal DNA.

“Sample genetic material” and “test genetic material” are equivalent terms as used herein which refer to genetic material from a CLL patient, particularly a patient for which an assessment of genomic alterations for the determination of a prognosis is desired. Such sample genetic material or test genetic material may be referred to herein as “sample DNA” or “test DNA” when the genetic material comprises DNA. Furthermore, such sample genetic material or test genetic material can be obtained, for example, from a test sample (described below) from the CLL patient.

“Reference genetic material” as used herein includes, for example, genetic material from one or more confirmed normal, healthy individuals, particularly one or more individuals that are not known to possess in the genomes one or more of the genomic alterations that are useful for determining the prognosis or risk stratification of a CLL patient as disclosed herein. Such reference genetic material may be referred to herein as reference DNA when the genetic material comprises DNA. Furthermore, such reference genetic material can be obtained, for example, from a reference sample (described below) from a normal, healthy individual. Reference genetic material also includes genetic material from normal tissue (i.e., non-cancerous cells) from a CLL patient (i.e., the sample genetic material or test material can be from the same individual as the reference genetic material).

As used herein, the term “label” is intended to mean any substance that can be attached to genetic material so that when the genetic material binds to a corresponding site a signal is emitted or the labeled genetic material can be detected by a human observer or an analytical instrument. Labels envisioned by the present invention can include any labels that emit a signal and allow for identification of a component in a sample or reference genetic material. Non-limiting examples of labels encompassed by the present invention include fluorescent moieties, radioactive moieties, chromogenic moieties, and enzymatic moieties.

Chromosome abnormalities are often associated with cancer, and genomic rearrangement, gain/amplification, deletion (loss), uniparental disomy, and mutation are alterations that can affect gene expression (and hence function) affecting multiple disease types, such as developmental syndromes and cancer. The detection and molecular definition of these alterations has stimulated research directed at understanding not only the functional role of the involved gene(s) in disease etiology but also in normal human biology.

As used herein, the term “copy number alteration” or “CNA” refers to the increase (i.e. genomic gain) or decrease (i.e. genomic loss) in the number of copies of all or any part of a chromosomal segment as compared to the “normal” or “standard” number of copies of all or any part of that chromosomal segment. Equivalent terms for “copy number alteration” include “copy number aberration” and “copy number variation.”

As used herein, “gain” of a chromosomal segment (e.g., “gain of 3q” or “3q gain”) refers to multiplication (amplification) of all or any part thereof of the chromosomal segment resulting in increased copy number of the segment. For example, “gain of 3q” can be multiplication (amplification) within 3q26. In some embodiments, gain of a chromosomal segment is determined by assessing whether a region defined by coordinates specified as peak limits in Table 5 has been gained.

As used herein, “loss” of a chromosomal segment (e.g., “loss of 3q” or “3q loss”) refers to a deletion of all or any part thereof of the chromosomal segment resulting in decreased copy number of the segment. In some embodiments, loss of a chromosomal segment is determined by assessing whether a region defined by coordinates specified as peak limits in Table 5 has been lost. In some embodiments, loss of 7q is determined by assessing whether a region defined by the following coordinates has been lost: chr7:122,471,896-124,803,693. In some embodiments, loss of 5p is determined by assessing whether a region defined by the following coordinates has been lost: chr5:5,460,990-8,079,142.

As used herein, the term “prognosis” refers to a prediction of the probable course and/or outcome of a clinical condition or disease. A prognosis of a patient is usually made by evaluating factors or symptoms of a disease that are indicative of a favorable or unfavorable course or outcome of the disease. It is recognized that a prognosis is a prediction of the course or outcome of a condition or disease and thus will not accurately predict the disease course or outcome for every CLL patient. Instead, the term prognosis refers to an increased probability that a certain course or outcome will occur; that is, that a course or outcome is more likely to occur in a patient exhibiting a given condition when compared to those individuals not exhibiting the condition. Examples of prognoses include predicting the time to first treatment, predicting overall survival, predicting response to therapy, predicting disease-free survival (i.e., living free of the disease), predicting progression-free survival (i.e., the length of time in which a patient is living with a disease that does not get worse), or predicting event-free survival (i.e., living without the occurrence of a particular group of defined events). The prognosis of a patient can be considered as an expression of relativism (e.g., prognostic groups based on relative time to first treatment or overall survival), with many factors affecting the ultimate outcome. For example, a patient with a poor prognosis might have a predicted shorter time to first treatment than a patient with a good prognosis.

Patients can be stratified into one of at least three prognostic groups using the methods disclosed herein: good prognosis, intermediate prognosis, and poor prognosis. As used herein, the term “poor prognosis” refers to a probable outcome that would be regarded as negative for a patient as compared to the probable outcome for patients in the “intermediate prognosis” and “good prognosis” groups. For example, a “poor prognosis” can be a probable shorter time to first treatment or overall survival as compared to patients in the “intermediate prognosis” and “good prognosis” groups.

As used herein, the term “good prognosis” refers to a probable outcome that would be regarded as positive for the patient as compared to the probable outcome for patients in the “intermediate prognosis” and “good prognosis” groups. For example, a “poor prognosis” can be a probable longer time to first treatment or overall survival as compared to patients in the “intermediate prognosis” and “good prognosis” groups.

As used herein, the term “intermediate prognosis” refers to a probable outcome that would be regarded as positive for the patient as compared to the probable outcome for patients in the “poor prognosis” group but would be regarded as negative for the patient as compared to the probable outcome for patients in the “good prognosis” group.

Patients within any one of the good, intermediate, and poor prognosis groups can be further stratified into “worse prognosis” and “better prognosis” groups. As used herein, the term “worse prognosis” refers to a probable outcome that would be regarded as negative for a patient as compared to the probable outcome for patients in the “better prognosis” group. For example, a “worse prognosis” can be a probable shorter time to first treatment or overall survival as compared to patients in the “better prognosis” group.

As used herein, the term “better prognosis” refers to a probable outcome that would be regarded as positive for a patient as compared to the probable outcome for patients in the “worse prognosis” group. For example, a “better prognosis” can be a probable longer time to first treatment or overall survival as compared to patients in the “worse prognosis” group.

As used herein, the term “time to first treatment” or “TTFT” refers to the time between the date of diagnosis of a CLL patient and the date of initiation of first treatment. In specific embodiments, the first treatment comprises chemotherapeutic or immunochemotherapeutic treatment.

As used herein, the term “overall survival” or “OS” refers to the time between the date of diagnosis of a CLL patient and the date of the death of the patient. The date of death can be the date of disease-related death and/or the date of death from other causes.

The present invention provides methods and systems that are useful in the prognosis of chronic lymphocytic leukemia (CLL). The methods and systems are particularly beneficial because they can be used in new methodologies that utilize minimal available biopsy material, can be carried out with an analyte that is stable, and are less invasive than known procedures for diagnostic/prognostic purposes.

In one aspect, the invention provides a system for risk stratification one or more chronic lymphocyte leukemia (CLL) patients or for stratifying one or more CLL patients into one or more CLL prognostic groups. In certain embodiments, the one or more CLL patients are treatment-naïve patients.

In some embodiments, the system comprises a microarray. In certain embodiments, the microarray can employ array comparative genomic hybridization (array-CGH or aCGH) to assist in the detection of CNAs. Comparative genomic hybridization is described, for example, in U.S. Pat. Nos. 5,665,549; 5,721,098; 6,159,685; 7,238,484; and 7,537,895; all of which are herein incorporated by reference in their entirety for all purposes. Array-CGH involves the simultaneous hybridization of differentially labeled test and reference DNAs to a microarray (BAC or oligonucleotide-based) representative of the entire genome or parts thereof. In one embodiment of the invention, test DNA can be labeled with Cy5-dUTP (red) and reference DNA is labeled with Cy3-dUTP (green). Following hybridization and scanning, BAC/oligonucleotide probes exhibiting increased red fluorescent signal over green is reflective of increased copy number of the sequence in the test DNA relative to the reference DNA (gain or amplification), increased green signal of decreased copy number in test DNA relative to reference DNA (loss), and yellow of no copy number change in the test DNA relative to the reference DNA. Array-CGH is a useful diagnostic tool because it can utilize DNA from fresh, frozen, or formalin-fixed paraffin-embedded (FFPE) specimens and can, in array format, detect genomic gain/loss at a large number of chromosomal loci at one time.

In particular embodiments, the system can comprise a specific oligonucleotide-based array that is useful in prognosis of chronic lymphocytic leukemia (CLL). Such arrays are described in, for example, U.S. Pat. Nos. 8,557,747 and 8,580,713, both of which are herein incorporated by reference in their entirety for all purposes. Such specific oligo-based arrays can, for example, represent a plurality of distinct genomic regions that exhibit an alteration therein (e.g., gain and/or loss) in chronic lymphocytic leukemias and can be used in varying techniques, platforms, and statistical algorithms. In specific embodiments, the array can be a Mature B-cell Neoplasm Array (MatBA®).

In certain embodiments, the microarray can be an oligonucleotide array and can comprise DNA arrayed thereon corresponding to at least one genomic region wherein an alteration in the genomic region is consistent with one or more CLL prognostic groups. More particularly, the genomic regions represented on the microarray can be regions wherein a copy number alteration (CNA) (e.g., gain, loss, or both gain and loss) in the region is consistent with one or more specific CLL prognostic groups. In other words, the genomic regions included in the microarray can be regions wherein genomic CNAs are shown to correlate with one or more specific CLL prognostic groups.

In one embodiment, a microarray in a system according to the invention can comprise a substrate with a plurality of distinct genomic regions arrayed thereon. Any substrate useful in forming diagnostic arrays can be used according to the present invention. For example, glass substrates, such as glass slides, can be used. Other non-limiting examples of useful substrates include silicon-based substrates, metal incorporating substrates (e.g., gold and metal oxides, such as titanium dioxide), gels, and polymeric materials. Useful substrates can be functionalized, such as to provide a specific charge, charge density, or functional group present at the substrate surface for immobilization of materials (e.g., oligonucleotides) to the substrate.

Preferably, each of the distinct genomic regions represented on the microarray is individually capable of hybridizing to material present in a test sample and/or reference sample. Preferably, the test sample is from a CLL patient, particularly a patient for which an assessment of genomic alterations for the determination of a prognosis is desired. In certain embodiments, the test sample can comprise all or part of a biopsy or biopsy specimen. In other embodiments, the test sample can comprise tissue that is fresh, frozen, or formalin-fixed paraffin-embedded (FFPE). In further embodiments, the test sample can comprise all or part of a blood or bone marrow specimen, including, for example, Ficoll-separated blood/bone marrow mononuclear cells (MNC). In further embodiments, the test sample can comprise all or part of a biopsy specimen, including, for example, tissue, core biopsy, or fine needle aspirate. The test sample particularly can comprise genetic material (i.e., sample genetic material). Preferably, the test sample comprises material in some form capable of hybridizing to the genomic regions represented on the microarray. In specific embodiments, the test sample can comprise DNA or fragments thereof.

Likewise, in certain embodiments, the reference sample can comprise all or part of a biopsy or biopsy specimen from, for example, normal healthy individual. In other embodiments, the reference sample can comprise tissue that is fresh, frozen, or FFPE. In further embodiments, the reference sample can comprise all or part of a blood or bone marrow specimen, including, for example, Ficoll-separated blood/bone MNC. In further embodiments, the reference sample can comprise all or part of a biopsy specimen, including, for example, tissue, core biopsy, or fine needle aspirate. The reference sample particularly can comprise genetic material (i.e., reference genetic material). Preferably, the reference sample comprises material in some form capable of hybridizing to the genomic regions represented on the microarray. In specific embodiments, the reference sample can comprise DNA or fragments thereof.

In specific embodiments, the distinct genomic regions can be between about 0.3 Mbp to about 21.3 Mbp in size. In specific embodiments, the distinct genomic regions can be represented on the microarray at a resolution with an average density of about 5 kbp to about 100 kbp, about 10 kbp to about 60 kbp, about 20 kbp to about 50 kbp, or about 30 kbp to about 40 kbp. In some embodiments, the distinct genomic regions are represented on the microarray at a resolution with an average density of about 35 kbp. In other embodiments, the distinct genomic regions are represented on the microarray at a resolution with an average density of about 33 kbp, 34 kbp, 36 kbp, or 37 kbp.

In specific embodiments, the genomic regions represented on the microarray can be regions wherein a particular alteration therein is correlated to a specific CLL prognosis. The type of alteration identified can be any alteration, as otherwise described herein, that is correlated to a specific CLL prognosis. In specific embodiments, the alteration can be a copy number alteration, particularly a gain or a loss.

The microarray can provide a plurality of genomic regions, and the exact number of genomic regions can vary depending upon the desired use of the microarray, the desired specificity of the array, and other desired outcomes. Preferably, the microarray comprises a sufficient number of genomic regions to determine a specific prognosis for one or more CLL patients.

The microarray can comprise only a single genomic region useful to determine the prognosis of one or more CLL patients. For example, the microarray can comprise or consist essentially of genomic regions comprising, consisting essentially of, or consisting of all or part of one or more of the following genomic regions: 2p, 3q, 8q, 17q, 7q, 8p, 11q, 17p, 18p, 13q14, 1p, 7p, 12, 18q, 19, 4p, 5p, and 6q. In some embodiments, the microarray comprises or consists essentially of one or more of the following genomic regions: all or part of 3q, all or part of 8q, all or part of 8p, all or part of 11q, or all or part of 17p. Preferably, the microarray comprises or consists essentially of more than one genomic region useful to determine the prognosis of one or more CLL patients. In certain embodiments, the microarray can comprise or consist essentially of a plurality of genomic regions that each can be useful for risk stratification of one or more CLL patients. As some genomic regions that may be used according to the invention can correlate to different CLL prognostic groups, it can be useful according to the invention for the microarray to include many different genomic regions having different alterations that correlate to specific CLL prognostic groups to assist in interpretation of signaling to determine the appropriate CLL prognosis for a given test sample.

The exact number of different genomic regions represented on the microarray can vary based upon the desired outcome of the test in which the array may be used. In specific embodiments, a single microarray according to the invention can comprise or consist essentially of at least 1 genomic region, at least 2 different genomic regions, at least 5 different genomic regions, at least 10 different genomic regions, at least 15 different genomic regions, at least 20 different genomic regions, at least 25 different genomic regions, at least 30 different genomic regions, at least 35 different genomic regions, at least 40 different genomic regions, at least 45 different genomic regions, at least 50 different genomic regions, at least 55 different genomic regions, at least 60 different genomic regions, at least 65 different genomic regions, at least 70 different genomic regions, at least 75 different genomic regions, or at least 80 different genomic regions. A microarray designed to detect only one CLL prognostic groups can use a smaller number of different genomic regions, while a microarray designed to detect many different CLL prognostic groups (e.g., 2, 3, 4, 5, or more) could include a much larger number of different genomic regions. Further, each different genomic region can be included in the array in multiple copies. The total number of genomic regions provided on a single microarray according to the invention thus can be greater than about 100, greater than about 250, greater than about 500, greater than about 1,000, greater than about 2,500, greater than about 5,000, greater than about 10,000, greater than about 15,000, greater than about 20,000, greater than about 25,000, greater than about 30,000, greater than about 35,000, greater than about 40,000, greater than about 45,000, or greater than about 50,000. In certain embodiments, the total number of genomic regions provided on a single microarray can be at least 2, 3, 4, 5, 6, 7, 8, 9, 10, 11, 12, 13, 14, 15, 16, 17, 18, 19, 20, 25, 30, 35, 40, 45, 50, 60, 70, 80, 90, or more different genomic regions.

In specific embodiments, the genomic regions represented on the microarray can include genomic regions comprising all or part of each of the following: (a) the genomic regions identified in Table 5; (b) 7q; and (c) 5p15. In other embodiments, the genomic regions represented on the microarray can consist essentially of the above regions. In yet other embodiments, the genomic regions represented on the microarray can consist of the above regions.

In specific embodiments, the genomic regions represented on the microarray can include genomic regions comprising each of the following: (a) regions defined by the coordinates specified as peak limits for each of the genomic regions identified in Table 5; (b) chr7:122,471,896-124,803,693; and (c) chr5:5,460,990-8,079,142. In other embodiments, the genomic regions represented on the microarray can consist essentially of the above regions. In yet other embodiments, the genomic regions represented on the microarray can consist of the above regions.

In specific embodiments, the genomic regions represented on the microarray can include all or part of each of the following genomic regions: 2p, 3q, 8q, 17q, 7q, 8p, 11q, 17p, 18p, 13q14, 1p, 7p, 12, 18q, 19, 4p, 5p, and 6q. In other embodiments, the genomic regions represented on the microarray can consist essentially of the above regions. In yet other embodiments, the genomic regions represented on the microarray can consist of the above regions.

In specific embodiments, the genomic regions represented on the microarray can include the genomic regions listed in Table 2 or genomic regions comprising all or part of each of the genomic regions listed in Table 2. In other embodiments, the genomic regions represented on the microarray can consist essentially of the above regions. In yet other embodiments, the genomic regions represented on the microarray can consist of the above regions.

In certain other embodiments, the genomic regions represented on the microarray can be identified in relation to chromosomal bands, although the region represented on the array need not necessarily include the entire band. Particularly, the plurality of genomic regions can comprise at least one chromosomal band selected from the groups shown in Tables 2 and 5 provided herein. In addition to varying based upon the different regions that may be represented on the microarray, the microarray in the system of the present invention can also vary based upon probe density within specific regions and multiplicity of arrayed oligonucleotides.

As evident from above, a microarray can be designed to incorporate genomic regions wherein a specific alteration, such as a gain or loss, correlates genetic material hybridized (e.g., DNA or fragments thereof) therewith to a specific prognosis of the respective CLL patient. Because of the identification of a large number of different genomic regions that correlate to a number of different CLL prognostic groups, it is possible according to the invention to provide a single array (e.g., a single chip or a single slide) to which a test sample can be applied and determine the prognosis of the patient from which the biopsy was derived.

In addition to the genomic regions described above that are present on the substrate, the microarray can also comprise one or more probes that may be useful for normalization of test results or to use as a comparative for analytical purposes. In some embodiments, for example, a backbone probe set may be used that covers the entire chromosomal complement. Such a backbone probe set may comprise varying numbers of probes at varying levels of resolution and preferably excludes regions of known copy number variation. In specific embodiments, such a backbone probe set may cover the entire chromosomal complement of a member of the animal kingdom that may be inflicted with CLL. In specific embodiments, such a backbone probe set may cover the entire chromosomal complement of a mammal that may be inflicted with CLL. In specific embodiments, such a backbone probe set may cover the entire human chromosomal complement. In specific embodiments, such a backbone probe set may cover the entire chromosomal complement at a resolution with an average density of about 1 Mbp.

In certain embodiments, the system comprises a decision tree or model comprising steps for stratification of one or more CLL patients into prognostic groups.

In certain embodiments, the decision tree comprises, consists essentially of, or consists of steps for stratification of each of one or more CLL patients into the following groups: (a) poor prognosis: the CLL patients whose sample genetic material comprises at least one of gain of 2p, gain of 3q, gain of 8q, gain of 17q, loss of 7q, loss of 8p, loss of 11q, loss of 17p, and loss of 18p; (b) good prognosis: the CLL patients whose sample genetic material comprises loss of 13q14 without any of the copy number alterations listed in step (a) and without any of gain of 1p, gain of 7p, gain of 12, gain of 18p, gain of 18q, gain of 19, loss of 4p, loss of 5p, loss of 6q, and loss of 7p; and (c) intermediate prognosis: all other CLL patients.

In certain embodiments, the first step is determining whether a CLL patient is in the poor prognostic group. If the patient is not in the poor prognostic group, the next step is determining whether the patient is in the good prognostic group. If the CLL patient is in neither the poor prognostic group nor the good prognostic group, the CLL patient is in the intermediate prognostic group.

In certain embodiments, the gains or losses in the steps for stratification are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

In certain embodiments, the decision tree further comprises steps for stratification of the CLL patients in the good prognosis and intermediate prognosis groups based on IGHV mutation status, wherein mutated IGHV predicts a better prognosis and unmutated IGHV predicts a worse prognosis. In certain embodiments, the decision tree further comprises steps based on other prognostic factors currently used in the medical field. Prognostication of CLL can also comprise the use of clinical features such as stage, expression of markers such as CD38 and ZAP-70 (by flow cytometry), IGHV mutation status (by PCR and sequencing), karyotype analysis, and fluorescence in situ hybridization (FISH) for the detection of gain or loss of four specific loci (13q, 11q, 17p, and 12) (see Shanafelt et al., Blood 103:1202-1210 (2004); Hallek et al., Blood 111:5446-5456 (2008)).

In certain embodiments, the decision tree is embodied in a written medium. In certain embodiments, the decision tree is embodied in a computer-readable medium. The computer-readable medium can have computer-executable code recorded thereon. The computer-readable medium can be any available tangible medium that can be accessed by a computer. Computer readable media include volatile and nonvolatile, removable and non-removable tangible media implemented in any method or technology for storage of information such as computer readable instructions, data structures, program modules, or other data. Computer-readable media include, but are not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and nonvolatile memory, and any other tangible medium that can be used to store the desired information and that can accessed by a computer including any suitable combination of the foregoing. In some embodiments, the computer-readable medium can include the “cloud” system, in which a user can store data on a remote server and later access the data or perform further analysis of the data from the remote server. The computer-readable media can be transportable such that the instructions stored thereon can be loaded onto any computer resource to implement the methods described herein.

In one embodiment, the computer-readable medium is software. Software includes, for example, instructions, data, or any combination thereof, whether referred to as software, firmware, middleware, microcode, hardware description, or otherwise. Instructions can include code in any format such as in source code format, binary code format, executable code format, or any other suitable format of code.

In some embodiments, the prognosis can be predicted time to first treatment, predicted overall survival, predicted response to therapy, predicted disease-free survival, predicted progression-free survival, and/or predicted event-free survival. In certain embodiments, the prognosis can be predicted time to first treatment and/or predicted overall survival. In other embodiments, the prognosis can be predicted time to first treatment. In yet other embodiments, the prognosis can be predicted overall survival.

In a further aspect, the present invention provides methods for risk stratification of one or more chronic lymphocytic leukemia (CLL) patients. Table 6 shows correlations between specific CNAs at specific genomic regions and various prognostic outcomes. A person skilled in the art using the present disclosure would be able to identify even further correlations between alterations at specific genomic regions and the same or other prognostic outcomes and thus could apply the presently described methods and devices in even further applications. Such further applications are intended to be encompassed by the present invention.

In some embodiments, a method for risk stratification of one or more CLL patients can comprise using one or more of the following technologies to detect CNAs in the CLL patients: karyotyping, spectral karyotyping (SKY), chromosomal comparative genomic hybridization (chromosomal-CGH), FISH, multiplex FISH (M-FISH), array-CGH, single nucleotide polymorphism array (SNP-array) analysis, polymerase chain reaction (PCR), and Southern blotting. In a clinical diagnostic setting, karyotyping, FISH, PCR, and to a much reduced extent Southern blotting, have been the technologies of choice, and the American College of Medical Genetics (ACMG) has established Standards and Guidelines for these technologies. Table 1 shows examples of technologies that are used for the examination of chromosome abnormalities with differing technical advantages and disadvantages (Bejjani and Shaffer (2008) Annu. Rev. Genomics Hum. Genet., 9:71-86.

TABLE 1
Common Technologies for Genomic Aberration Detection.

| Technique | Resolution | | Coverage | Aberrations Detected |
|---|---|---|---|---|
| Karyotype | >10 | Mbp | Whole genome | Rearrangement (balanced, unbalanced), |
| | | | | gain, loss |
| SKY | >2 | Mbp | Whole genome | Rearrangement (balanced, unbalanced), |
| | | | | gain, loss |
| Chromosomal-CGH | >2 | Mbp | Whole genome | Gain, loss |
| FISH | >20 | kbp | Probe-specific | Rearrangement (balanced, unbalanced), |
| | | | | gain, loss |
| Array-CGH | 5-100 | kbp* | Whole genome | Rearrangement (unbalanced), gain, loss |
| SNP-Array | 5 | kbp | Whole genome | Gain, loss, uniparental disomy, |
| | | | | mutation |
| PCR | <10 | | kbp | Gene-specific |
| Southern Blotting | <20 | | kbp | Gene-specific |

In some embodiments, a method for risk stratification of one or more CLL patients can comprise using next-generation sequencing to detect CNAs in the CLL patients. See, e.g., Wood et al. (2010) Nucleic Acids Res. 38:e151; Sobreira et al. (2011) Genome Research 21:1720-1727; Vergult et al. (2014) Eur. J. Hum. Genet. 22:652-659. The term “next-generation sequencing” includes sequencing methods that allow for massively parallel sequencing of clonally amplified molecules and of single nucleic acid molecules. Next-generation sequencing can also be referred to as “NGS” or “massively parallel sequencing” or “high throughput sequencing.” Non-limiting examples of next-generation sequencing include sequencing-by-synthesis using reversible dye terminators and sequencing-by-ligation (e.g., platforms employed by Illumina, Life Technologies, and Roche). Next-generation sequencing methods also include nanopore sequencing methods or electronic-detection-based methods such as Ion Torrent technology commercialized by Life Technologies. Specific examples of next-generation sequencing include massively parallel signature sequencing (MPSS), polony sequencing, 454 pyrosequencing, Illumina (Solexa) sequencing, SOLiD sequencing, ion semiconductor sequencing, DNA nanoball sequencing, Helioscope™ single molecule sequencing, single molecule SMRT™ sequencing, single molecule real-time (RNAP) sequencing, and nanopore DNA sequencing. In one embodiment, next-generation sequencing can detect CNAs by comparing the number of sequence reads in non-overlapping windows between sample genetic material from a CLL patient and control reference genetic material.

In some embodiments, a method for risk stratification of one or more CLL patients can comprise providing a system as otherwise described herein. The present invention encompasses a number of different variations of systems noted above, and all such systems could be used in the methods of the invention.

In some embodiments, a method for risk stratification of one or more CLL patients can comprise detecting the presence of copy number alterations in sample genetic material from each of said one or more patients. In some embodiments, the one or more CLL patients are treatment-naïve patients.

In further embodiments, the methods of the invention may comprise, consist essentially of, or consist of stratifying each of the one or more CLL patients into one of the following groups: (i) poor prognosis: the CLL patients whose sample genetic material comprises at least one of gain of 2p, gain of 3q, gain of 8q, gain of 17q, loss of 7q, loss of 8p, loss of 11q, loss of 17p, and loss of 18p; (ii) good prognosis: the CLL patients whose sample genetic material comprises loss of 13q14 without any of the copy number alterations listed in step (i) and without any of gain of 1p, gain of 7p, gain of 12, gain of 18p, gain of 18q, gain of 19, loss of 4p, loss of 5p, loss of 6q, and loss of 7p; and (iii) intermediate prognosis: all other CLL patients.

In certain embodiments, the first step is determining whether a CLL patient is in the poor prognostic group. If the patient is not in the poor prognostic group, the next step is determining whether the patient is in the good prognostic group. If the CLL patient is in neither the poor prognostic group nor the good prognostic group, the CLL patient is in the intermediate prognostic group.

In certain embodiments, the gains or losses in the steps for stratification are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

In some embodiments, the prognosis can be predicted time to first treatment, predicted overall survival, predicted response to therapy, predicted disease-free survival, predicted progression-free survival, and/or predicted event-free survival. In some embodiments, the poor prognosis is shorter predicted time to first treatment and/or shorter predicted overall survival and the good prognosis is longer predicted time to first treatment and/or longer predicted overall survival. In other embodiments, the poor prognosis is shorter predicted time to first treatment and the good prognosis is longer predicted time to first treatment. In yet other embodiments, the poor prognosis is shorter predicted overall survival and the good prognosis is longer predicted overall survival.

In other embodiments, the methods may comprise further stratifying the CLL patients in the good prognosis and intermediate prognosis groups based on IGHV mutation status. In some embodiments, mutated IGHV predicts a better prognosis and unmutated IGHV predicts a worse prognosis. In certain embodiments, the methods may comprise further stratifying the CLL patients based on other prognostic factors currently used in the medical field. Prognostication of CLL can also comprise the use of clinical features such as stage, expression of markers such as CD38 and ZAP-70 (by flow cytometry), IGHV mutation status (by PCR and sequencing), karyotype analysis, and fluorescence in situ hybridization (FISH) for the detection of gain or loss of four specific loci (13q, 11q, 17p, and 12) (see Shanafelt et al., Blood 103:1202-1210 (2004); Hallek et al., Blood 111:5446-5456 (2008); Dohner et al., N. Engl. J. Med. 343:1910-1916 (2000)).

In some embodiments, the worse prognosis is shorter time to first treatment and/or shorter overall survival and the better prognosis is longer time to first treatment and/or longer overall survival. In other embodiments, the worse prognosis is shorter time to first treatment and the better prognosis is longer time to first treatment. In yet other embodiments, the worse prognosis is shorter overall survival and the better prognosis is longer overall survival.

In some embodiments, a method for risk stratification of one or more chronic lymphocytic leukemia patients can comprise providing a microarray as otherwise described herein. As noted above, the present invention encompasses a number of different variations of microarrays and all such microarrays can be used in the methods of the present invention.

In certain embodiments, the methods can comprise providing a sample (e.g., test sample or reference sample) with genetic material therein. In certain embodiments, the genetic material can be labeled. In carrying out the methods of the invention, a sample for testing may be provided in a form wherein any genetic material present in the test sample already has been subjected to a labeling procedure to provide labels suitable for use according to the invention. In other embodiments, the methods can comprise the actual step of labeling the genetic material present in the sample. Any method suitable for labeling of genetic material, such as DNA, may be used according to the invention. For example, the DNA could be digested with a suitable material, such as Rsa I and/or Alu I, and then appropriately labeled. In one embodiment, fluorescent labeling may be used (such as, for example, Cyanine 5-dUTP (Cy5) or Cyanine 3-dUTP (Cy3) using Klenow DNA polymerase).

In some embodiments, labeled test genetic material (i.e., labeled sample genetic material) is provided. In some embodiments, labeled reference genetic material is provided in addition to the labeled test genetic material. Such reference genetic material can include, for example, genetic material from confirmed normal healthy individuals.

The methods of the invention can further comprise hybridizing the genetic materials (test sample and/or reference sample) with the genomic regions represented on the microarray. Any hybridization method useful in the art could be used in hybridizing the genetic materials with the genomic regions. One method could encompass combining the genetic materials, human Cot-1, a blocking agent, and a hybridization buffer, and allowing the genetic materials to hybridize with the genomic regions on the microarray for a sufficient time (e.g., about 24 hours) under acceptable conditions (e.g., a temperature of about 65° C.). Hybridization kits and techniques commercially available, such as from Agilent Technologies, could be used.

In some embodiments, the genetic materials (test and/or reference) are further hybridized with a backbone probe set arrayed on the substrate. Such a backbone probe set can be any of the backbone probe sets described above.

In some embodiments, reference genetic material is also hybridized with the genomic regions represented on the microarray (i.e., arrayed on the substrate). In some embodiments, the reference genetic material is further hybridized with the backbone probe set arrayed on the substrate.

In some embodiments, the methods can further comprise analyzing the hybridization pattern of the genetic materials (test and/or reference) to the genomic regions. Analyzing methods useful according to the present invention can vary depending upon the type of labeling used on the genetic materials. Preferably, analyzing can be carried out using equipment useful to evaluate hybridization patterns and to identify regions on the microarray where alterations in the test sample occur.

In some embodiments, the hybridization pattern of reference genetic material is analyzed in addition to the hybridization pattern of sample genetic material. In some embodiments, the methods further comprise analyzing the hybridization pattern of the sample genetic material to the distinct genomic regions relative to the hybridization pattern of the reference genetic material to the distinct genomic regions to detect the presence of copy number alterations in the sample genetic material. Such analysis can be useful to detect the presence of alterations in the genetic material from the sample relative to the reference genetic material. In some embodiments, the sample genetic material and the reference genetic material are hybridized with the distinct genomic regions represented on the microarray at the same time. In a preferred embodiment of the invention, the sample genetic material comprises a first label and the reference genetic material comprises a second label, and the first and second labels are non-identical and can be detected simultaneously when hybridized to at least one of the distinct genomic regions represented on the microarray.

In certain embodiments, the methods of the invention analyzing the hybridization pattern can involve imaging a microarray such as, for example, the imaging methods described in U.S. Pat. No. 7,636,636; herein incorporated by reference in its entirety for all purposes. Such methods can involve, for example, acquiring an image of a microarray including, for example, a target spot; processing the image to correct for background noise and chip misalignment; analyzing the image to detect target spots; analyzing the image to identify the target patch, editing debris and correcting for ratio bias; detecting number variation in the target spot by an objective statistical analysis, wherein the sample genetic material and the reference genetic material form the target spot by the hybridizing; measuring a fluorescent signal intensity of the target spot from the sample genetic material and the reference genetic material; obtaining an image; and cross-correlating the image to the image of the microarray. Such imaging methods typically the use of computer programs for analyzing the imaged microarrays. See e.g., U.S. Pat. No. 7,636,636.

### Embodiments of the Invention

Embodiments of the invention include, but are not limited to, the following embodiments:

1. A method for risk stratification of a chronic lymphocytic leukemia (CLL) patient, the method comprising, consisting essentially of, or consisting of:
(a) detecting the presence of copy number alterations in sample genetic material from said CLL patient; and
(b) stratifying said CLL patient into one of the following groups:
   (i) poor prognosis: CLL patients whose sample genetic material comprises at least one of gain of 2p, gain of 3q, gain of 8q, gain of 17q, loss of 7q, loss of 8p, loss of 11q, loss of 17p, and loss of 18p;
   (ii) good prognosis: CLL patients whose sample genetic material comprises loss of 13q14 without any of the copy number alterations listed in step (b)(i) and without any of gain of 1p, gain of 7p, gain of 12, gain of 18p, gain of 18q, gain of 19, loss of 4p, loss of 5p, loss of 6q, and loss of 7p; and
   (iii) intermediate prognosis: all other CLL patients.

2. The method of embodiment 1, wherein step (b)(i) occurs before step (b)(ii), and step (b)(ii) occurs before step (b)(iii).

3. The method of embodiment 1 or 2, wherein the gains or losses in step (b) are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

4. The method of any preceding embodiment, wherein the detecting step comprises, consists essentially of, or consist of one or more of array-based comparative genomic hybridization (aCGH), next-generation sequencing, karyotyping, spectral karyotyping (SKY), chromosomal comparative genomic hybridization (chromosomal-CGH), fluorescence in situ hybridization (FISH), multiplex FISH (M-FISH), single nucleotide polymorphism array (SNP-array) analysis, polymerase chain reaction (PCR), and Southern blotting.

5. The method of any preceding embodiment, wherein said CLL patient is a human CLL patient.

6. The method of any preceding embodiment, wherein said poor prognosis is shorter predicted time to first treatment and/or shorter predicted overall survival and said good prognosis is longer predicted time to first treatment and/or longer predicted overall survival.

7. The method of embodiment 6, wherein said poor prognosis is shorter predicted time to first treatment and wherein said good prognosis is longer predicted time to first treatment.

8. The method of embodiment 6, wherein said poor prognosis is shorter predicted overall survival and wherein said good prognosis is longer predicted overall survival.

9. The method of any preceding embodiment, further comprising further stratifying said CLL patient based on IGHV mutation status, wherein mutated IGHV predicts a better prognosis and unmutated IGHV predicts a worse prognosis for CLL patients in the good prognosis and intermediate prognosis groups.

10. The method of embodiment 9, wherein said worse prognosis is shorter predicted time to first treatment and/or shorter predicted overall survival and said better prognosis is longer predicted time to first treatment and/or longer predicted overall survival.

11. The method of embodiment 10, wherein said worse prognosis is shorter predicted time to first treatment and wherein said better prognosis is longer predicted time to first treatment.

12. The method of embodiment 10, wherein said worse prognosis is shorter predicted overall survival and wherein said better prognosis is longer predicted overall survival.

13. The method of any preceding embodiment, wherein said CLL patient is a treatment-naïve patient.

14. The method of any preceding embodiment, wherein the detecting step comprises, consists essentially of, or consists of:
(i) providing a microarray, said microarray comprising a substrate comprising a plurality of distinct genomic regions arrayed thereon;
(ii) providing said sample genetic material;
(iii) hybridizing said sample genetic material with said distinct genomic regions arrayed on said substrate; and
(iv) analyzing the hybridization pattern of said sample genetic material to said distinct genomic regions to detect the presence of copy number alterations in said sample genetic material.

15. The method of embodiment 14, wherein said sample genetic material is labeled sample genetic material.

16. The method of embodiment 14 or 15, wherein said distinct genomic regions comprise genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

17. The method of embodiment 16, wherein said distinct genomic regions consist essentially of genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

18. The method of embodiment 17, wherein said distinct genomic regions consist of genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

19. The method of embodiment 14 or 15, wherein said distinct genomic regions comprise genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

20. The method of embodiment 19, wherein said distinct genomic regions consist essentially of genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

21. The method of embodiment 20, wherein said distinct genomic regions consist of genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

22. The method of embodiment 14 or 15, wherein said distinct genomic regions comprise genomic regions comprising, consisting essentially of, or consisting of all or part of each of the genomic regions listed in Table 2.

23. The method of embodiment 22, wherein said distinct genomic regions consist essentially of genomic regions comprising, consisting essentially of, or consisting of all or part of each of the genomic regions listed in Table 2.

24. The method of embodiment 23, wherein said distinct genomic regions consist of genomic regions comprising, consisting essentially of, or consisting of all or part of each of the genomic regions listed in Table 2.

25. The method of embodiment 22, wherein said distinct genomic regions comprise the genomic regions listed in Table 2.

26. The method of embodiment 25, wherein said distinct genomic regions consist essentially of the genomic regions listed in Table 2.

27. The method of embodiment 26, wherein said distinct genomic regions consist of the genomic regions listed in Table 2.

28. The method of any one of embodiments 14-27, wherein each of said distinct genomic regions is individually capable of hybridizing to material present in said sample genetic material.

29. The method of any one of embodiments 14-28, wherein said distinct genomic regions are between about 0.3 Mbp to about 21.3 Mbp in size and are represented on said microarray at a resolution with an average density of about 35 kbp.

30. The method of any one of embodiments 14-29, wherein the providing step further comprises providing reference genetic material, wherein the hybridizing step further comprises hybridizing said reference genetic material with said distinct genomic regions arrayed on said substrate, and wherein the analyzing step further comprises analyzing the hybridization pattern of said sample genetic material to said distinct genomic regions relative to the hybridization pattern of said reference genetic material to said distinct genomic regions to detect the presence of copy number alterations in said sample genetic material.

31. The method of embodiment 30, wherein said reference genetic material is labeled reference genetic material and said sample genetic material is labeled sample genetic material.

32. The method of embodiment 30 or 31, wherein said sample genetic material and said reference genetic material are hybridized with said distinct genomic regions arrayed on said substrate at the same time.

33. The method of embodiment 31 or 32, wherein said labeled sample genetic material comprises a first label and said labeled reference genetic material comprises a second label, wherein said first label and said second label are non-identical and can be detected simultaneously when hybridized to at least one of said distinct genomic regions arrayed on said substrate.

34. The method of any one of embodiments 14-29, wherein said substrate further comprises a backbone probe set arrayed thereon that covers the entire chromosomal complement, and wherein the hybridizing step further comprises hybridizing said sample genetic material with said backbone probe set arrayed on said substrate.

35. The method of any one of embodiments 30-33, wherein said substrate further comprises a backbone probe set arrayed thereon that covers the entire chromosomal complement, and wherein the hybridizing step further comprises hybridizing said sample genetic material and said reference genetic material with said backbone probe set arrayed on said substrate.

36. The method of embodiment 34 or 35, wherein said backbone probe set covers the entire chromosomal complement at a resolution with an average density of about 1 Mbp.

37. The method of any one of embodiments 34-36, wherein said backbone probe set excludes genomic regions of known copy number variation.

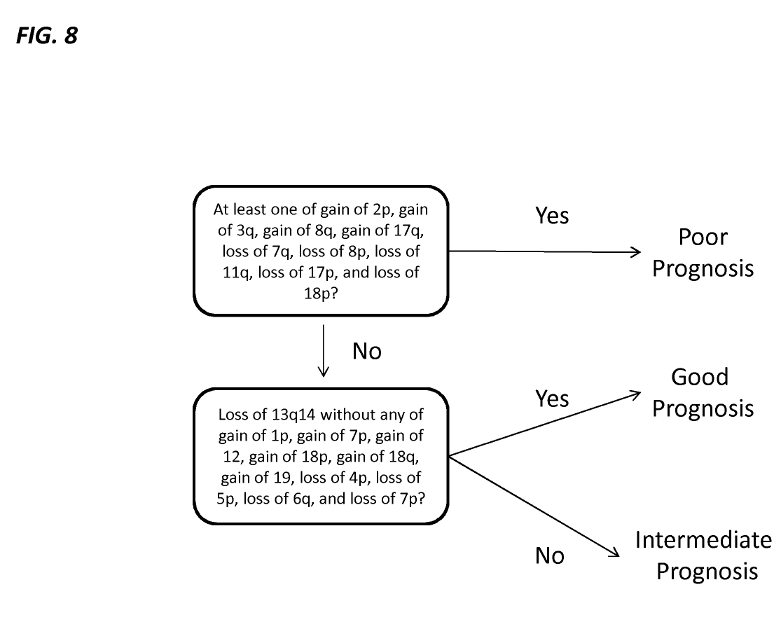

38. A system for risk stratification of a CLL patient, the system comprising, consisting essentially of, or consisting of a microarray and a decision tree comprising FIG. 8.

39. A system for risk stratification of a CLL patient, the system comprising, consisting essentially of, or consisting of a microarray and a decision tree comprising, consisting essentially of, or consisting of steps for stratification of said CLL patient into one of the following groups:
(a) poor prognosis: CLL patients whose sample genetic material comprises at least one of gain of 2p, gain of 3q, gain of 8q, gain of 17q, loss of 7q, loss of 8p, loss of 11q, loss of 17p, and loss of 18p;
(b) good prognosis: CLL patients whose sample genetic material comprises loss of 13q14 without any of the copy number alterations listed in step (a) and without any of gain of 1p, gain of 7p, gain of 12, gain of 18p, gain of 18q, gain of 19, loss of 4p, loss of 5p, loss of 6q, and loss of 7p; and
(c) intermediate prognosis: all other CLL patients.

40. The system of embodiment 39, wherein step (a) occurs before step (b), and step (b) occurs before step (c).

41. The system of any one of embodiments 38-40, wherein the gains or losses are determined by assessing gain or loss of the region defined by coordinates chr7:122,471,896-124,803,693 for 7q, the region defined by coordinates chr5:5,460,990-8,079,142 for 5p, and the regions defined by the coordinates specified as peak limits in Table 5 for the remainder of the copy number alterations.

42. The system of any one of embodiments 39-41, wherein said decision tree further comprises steps for stratification of said CLL patient based on IGHV mutation status, wherein mutated IGHV predicts a better prognosis and unmutated IGHV predicts a worse prognosis for CLL patients in the good prognosis and intermediate prognosis groups.

43. A system for risk stratification of a CLL patient, the system comprising, consisting essentially of, or consisting of a microarray and a decision tree comprising, consisting essentially of, or consisting of steps for stratifying said CLL patient according to step (b) from embodiment 1.

44. The system of embodiment 43, wherein said decision tree further comprises steps for stratifying said CLL patient according to embodiment 7.

45. The system of any one of embodiments 38-44, wherein said CLL patient is a human CLL patient.

46. The system of any one of embodiments 38-45, wherein said decision tree is embodied in a computer-readable medium.

47. The system of any one of embodiments 38-45, wherein said decision tree is embodied in a written medium.

48. The system of any one of embodiments 38-47, wherein the prognosis is predicted time to first treatment and/or predicted overall survival.

49. The system of embodiment 48, wherein the prognosis is predicted time to first treatment.

50. The system of embodiment 48, wherein the prognosis is predicted overall survival.

51. The system of any one of embodiments 38-50, wherein said CLL patient is a treatment-naïve patient.

52. The system of any one of embodiments 38-51, wherein said microarray comprises a substrate comprising a plurality of distinct genomic regions arrayed thereon.

53. The system of embodiment 52, wherein said distinct genomic regions comprise genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

54. The system of embodiment 53, wherein said distinct genomic regions consist essentially of genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

55. The system of embodiment 54, wherein said distinct genomic regions consist of genomic regions comprising, consisting essentially of, or consisting of all or part of:
(a) each of the genomic regions identified in Table 5 or regions defined by the coordinates specified as peak limits for each of the genomic regions identified in Table 5;
(b) 7q or the region between coordinates 122,471,896-124,803,693 on chromosome 7; and
(c) 5p15 or the region between coordinates 5,460,990-8,079,142 on chromosome 5.

56. The system of embodiment 52, wherein said distinct genomic regions comprise genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

57. The system of embodiment 56, wherein said distinct genomic regions consist essentially of genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

58. The system of embodiment 57, wherein said distinct genomic regions consist of genomic regions comprising, consisting essentially of, or consisting of all or part of each of the following genomic regions: 2p; 3q; 8q; 17q; 7q; 8p; 11q; 17p; 18p; 13q14; 1p; 7p; 12; 18q; 19; 4p; 5p; and 6q.

59. The system of embodiment 52, wherein said distinct genomic regions comprise genomic regions comprising, consisting essentially of, or consisting of all or part of each of the genomic regions listed in Table 2.

60. The system of embodiment 59, wherein said distinct genomic regions consist essentially of genomic regions comprising, consisting essentially of, or consisting of all or part of each of the genomic regions listed in Table 2.

61. The system of embodiment 60, wherein said distinct genomic regions consist of genomic regions comprising, consisting essentially of, or consisting of all or part of each of the genomic regions listed in Table 2.

62. The system of embodiment 59, wherein said distinct genomic regions comprise the genomic regions listed in Table 2.

63. The system of embodiment 62, wherein said distinct genomic regions consist essentially of the genomic regions listed in Table 2.

64. The system of embodiment 63, wherein said distinct genomic regions consist of the genomic regions listed in Table 2.

65. The system of any one of embodiments 56-64, wherein each of said distinct genomic regions is individually capable of hybridizing to material present in sample genetic material from said CLL patient.

66. The system of any one of embodiments 56-65, wherein said distinct genomic regions are between about 0.3 Mbp to about 21.3 Mbp in size and are represented on said microarray at a resolution with an average density of about 35 kbp.

67. The system of any one of embodiments 56-66, wherein said substrate further comprises a backbone probe set arrayed thereon that covers the entire chromosomal complement.

68. The system of embodiment 67, wherein said backbone probe set covers the entire chromosomal complement at a resolution with an average density of about 1 Mbp.

69. The system of embodiment 67 or 68, wherein said backbone probe set excludes genomic regions of known copy number variation.

70. Use of any one of the systems of embodiments 38-69 to determine the prognosis for a CLL patient.

71. Use of any one of the systems of embodiments 38-69 to determine the prognosis for a CLL patient by any one of the methods of embodiments 1-37.

### Examples

### Materials and Methods

### CLL Patient Specimens and DNA Extraction

Specimens (blood or bone marrow) were obtained from CLL patients with informed consent during routine care at the North Shore-LIJ Health System. Dataset 1 (DS1) comprised 119 cryopreserved mononuclear cells (MNC) isolated from CLL patients between 1998 and 2009, while Dataset 2 (DS2) comprised DNA extracted in the Cancer Genetics, Inc. Clinical Laboratory Improvement Amendments (CLIA)-approved laboratory from 169 blood/bone marrow specimens, consecutively ascertained during 2008 and 2009. Selection of cases was based on classification as CLL according to the World Health Organization (WHO) classification scheme (Swerdlow et al., WHO Classification of Tumours of Haematopoietic and Lymphoid Tissues, Lyon: IARC (2008)), and availability of a specimen (MNC or DNA) for study (see Supplemental Table I from Houldsworth et al., Leukemia & Lymphoma 55:920-928 (2014)). Across both datasets, 228 patients were untreated at the time of sampling and 60 were treated. DNA was also extracted from an independent validation dataset of cryopreserved MNCs from 65 similarly selected CLL specimens obtained from patients with consent at the Hackensack University Medical Center (HUMC). For six of these specimens CD19-immunomagnetic positive selection was performed prior to DNA extraction, on account of low absolute lymphocyte counts. Copy number data assessed using Affymetrix 6.0 SNP arrays were made available for 124 previously untreated prospectively enrolled CLL patients, performed with consent at the Dana Farber Cancer Institute (DFCI) (Brown et al., Clin. Cancer Res. 18:3791-3802 (2012)). All studies were performed with respective Institutional Review Board (IRB) approval.

Custom aCGH

The custom oligonucleotide array was designed within eArray (Agilent Technologies, Inc.) with a 4×44K format comprising 301 features (probes) represented five times to permit the assessment of reproducibility of each hybridization, a backbone of 3,100 features (oligonucleotides) in duplicate representing the entire genome at an average resolution of 1 Mbp, and 17,348 features (oligonucleotides) in duplicate representing eighty regions of the human genome ranging in size from 0.3 Mbp to 21.3 Mbp at an average resolution of 35 kbp (detailed below and also described in U.S. Pat. Nos. 8,557,747 and 8,580,713, both of which are herein incorporated by reference in their entirety for all purposes). Following aCGH as described in detail in below, data extraction was performed (Feature Extraction Version 10.7.3.1, Agilent), duplicate probes averaged, and the circular binary segmentation (CBS) method used to define segments (p=0.01) with the DNA copy package in R Bioconductor (Version 2.10). Genomic Identification of Significant Targets In Cancer (GISTIC, Version 0.9.2) was applied after removal of known normal copy number variants (Database of Genomic Variants, found at projects.tcag.ca/variation) with a minimum acceptable segment of eight contiguous probes and an acceptable false discovery rate (FDR) Q-value for significance of 0.25. For manual examination of aberrations in the CBS-segmented profiles, median-normalization was performed. Both for GISTIC and manual examination, specimens were scored positive with log ratios≧0.15 for gain and ≦−0.15 for loss as confirmed by quantitative polymerase chain reaction (QPCR) (described below). Raw data files for DS1 and DS2 have been deposited in Gene Expression Omnibus (GEO) (GSE40834). All genomic coordinates are according to the NCBI36/hg18 assembly.

### Array Design

The eighty regions represented on the custom array are listed in Table 2 according to NCBI36, Hg18.

TABLE 2
Genomic Regions Represented on MatBA

| | Location | Size |
|---|---|---|
| Band(s) | (Mbp) | (Mbp) |
| 1p36.32-p36.23 | 1.5-9.4 | 7.9 |
| 1p21 | 94.0-107 | 13 |
| 1p13.2-p13.1 | 111.6-117.6 | 6 |
| 1q21 | 142.4-153.3 | 10.9 |
| 1q31 | 184.6-204.3 | 19.7 |
| 1q41-q44 | 236.5-244.5 | 8 |
| 2p25.3 | 2.4-4.1 | 1.7 |
| 2p16.1-p15 | 59.3-63.9 | 4.6 |
| 2p11.2-q11.2 | 88-90 | 2 |
| 2q13-2q14.1 | 113.6-114.2 | 0.6 |
| 2q24 | 154.6-169.5 | 14.9 |
| 3p22 | 32.1-42 | 9.9 |
| 3p14.1-p13 | 69.9-73.7 | 3.8 |
| 3q12.2-q12.3 | 102.0-103.2 | 1.2 |
| 3q21.2 | 126.4-126.7 | 0.3 |
| 3q22 | 131.5-140.4 | 8.9 |
| 3q26.1-q26.2 | 161.2-172.5 | 11.3 |
| 3q26.31 | 173-175 | 2 |
| 3q27 | 184.2-189.4 | 5.2 |
| 4p15 | 24.9-34.7 | 9.8 |
| 4q11-q12 | 52-56 | 4 |
| 4q24 | 102-104.8 | 2.8 |
| 4q34.3-q35 | 178.3-189.9 | 11.6 |
| 5p15 | 0-10 | 10 |
| 5q13.2-5q13.3 | 73-76 | 3 |
| 5q31.3 | 140-141 | 1 |
| 6p25 | 0-7 | 7 |
| 6p21.31-p21.2 | 35.7-37.7 | 2 |
| 6p21.1 | 41-43 | 2 |
| 6q12 | 66.9-67.2 | 0.3 |
| 6q16 | 92-104.8 | 12.8 |
| 6q21 | 108-110.5 | 2.5 |
| 6q22 | 113.9-130.4 | 16.5 |
| 6q23.3-q24 | 137.2-149.1 | 11.9 |
| 6q25 | 152.2-153.2 | 1 |
| 7p22 | 0-7.2 | 7.2 |
| 7p21.3-p21.2 | 12.9-14.6 | 1.7 |
| 7q31 | 107.2-126.9 | 19.7 |
| 8p23 | 0-12.7 | 12.7 |
| 8p21.3 | 18.7-23.2 | 4.5 |
| 8p12-p11.23 | 37.4-39.4 | 2 |
| 8q21.2 | 86.6-86.9 | 0.3 |
| 8q24.21 | 127.3-131.5 | 4.2 |
| 9p24.2-p24.1 | 4.0-6.0 | 2 |
| 9p21 | 19.9-32.8 | 12.9 |
| 9q22 | 89.6-101.6 | 12 |
| 9q33.2-q34.1 | 122-132 | 10 |
| 10p14 | 6.7-12.3 | 5.6 |
| 10p12.31-p12.2 | 21.6-24.1 | 2.5 |
| 10q23.2 | 87.9-89.8 | 1.9 |
| 11p13 | 33.0-34.5 | 1.5 |
| 11q13 | 63.1-76.7 | 13.6 |
| 11q22.1-q22.2 | 100.8-102.2 | 1.4 |
| 11q22.3-q23 | 106.7-120.7 | 14 |
| 11q25 | 132.5-134.5 | 2 |
| 12p13.1 | 12.6-14.8 | 2.2 |
| 12q13.1-q13.2 | 44.6-56.5 | 11.9 |
| 12q15 | 66-69.8 | 3.8 |
| 13q14 | 39.5-52.2 | 12.7 |
| 13q31 | 77.8-93.8 | 16 |
| 13q33-q34 | 100.5-114.1 | 13.6 |
| 14q12 | 23.5-32.5 | 9 |
| 14q32 | 90.1-105.1 | 15 |
| 15q21.1 | 44.0-45.5 | 1.5 |
| 15q23-q24 | 65-75 | 10 |
| 16p13.3 | 0-6.3 | 6.3 |
| 16p13.13 | 10.3-12.3 | 2 |
| 16p11.1-p11.2 | 27.6-38.2 | 10.6 |
| 16q24 | 83.6-88.8 | 5.2 |
| 17p13 | 0-11.2 | 11.2 |
| 17q22-q23.1 | 53-55 | 2 |
| 17q24.2-25.1 | 64.1-69.9 | 5.8 |
| 18p11 | 0-16.1 | 16.1 |
| 18q21 | 41.8-59.8 | 18 |
| 18q23 | 75.1-75.4 | 0.3 |
| 19p13.3-p13.2 | 0.2-11.2 | 11 |
| 19q13.33-q13.43 | 53.8-63.8 | 10 |
| 20q13 | 41.1-62.4 | 21.3 |
| 21q21 | 15.3-30.5 | 15.2 |
| 22q12 | 20-35.9 | 15.9 |

aCGH Processing

DNA was extracted from DS1 and HUMC MNC specimens using the DNeasy Blood and Tissue Kit (QIAGEN) and considered of adequate quality for aCGH if the A260/A280 ratio was greater than or equal to 1.8 and if the A260/A230 ratio was greater or equal to 1.95. DNAs not meeting these criteria were further purified using the QIAquick PCR Purification Kit (QIAGEN). Restriction and differential labeling of CLL DNA (1 μg) and reference (MF) DNA (1 μg, equimixture male/female DNA, Promega Corp.) were performed essentially as recommended by the manufacturer (Agilent). Briefly, DNAs were digested with Rsa I and Alu I (Promega) and then labeled with Cyanine 5-dUTP (Cy5) or Cyanine 3-dUTP (Cy3) (Agilent) respectively using random primers and Klenow fragment (Agilent). Unincorporated Cy5 and Cy3 were removed and labeled DNA concentrated by centrifugation using Microcon YM-30 filter units (EMD Millipore Corp.). Prior to hybridization, Cot-1 DNA (5 μg, Life Technologies), blocking agent (Agilent), and 2× hybridization mix (Agilent) were added, followed by denaturation at 95° C. for 3 min and renaturation at 37° C. for 30 min. The slides containing four arrays were hybridized at 65° C. for 24 hours with constant rotation, and following washes (according to the manufacturer), were scanned using an Agilent Scanner.

Assessment of aCGH Sensitivity and Specificity Based on FISH Data

FISH data for the four commonly assessed loci was available for 103 specimens. When considering aberrations present in at least 25% of cells, the sensitivity of detection of aberrations by aCGH was 93.4% and specificity 98.8% for a total of 76 abnormal and 321 normal FISH results. Of the five aberrations not detected by aCGH, two were in specimens in which other aberrations were confirmed by both technologies. For four of the six aberrations discordantly detected by aCGH, the aberration was detected by FISH, but in less than 25% of cells (9-17%). For another, a separate FISH analysis performed within three months of the original sampling date, confirmed the aberration identified by aCGH. In the remaining case, a loss was detected by aCGH outside of the ATM locus detected by FISH.

### Confirmation of Aberrations by Quantitative PCR (QPCR)

QPCR was performed to confirm eight regional aCGH aberrations using the copy number assays provided below. In brief, 5 ng DNA per well were amplified in duplicate per gene per DNA, using TERT and RAG2 as control genes. The MET method was calculated using the average of the control genes for two independent equimixture male and female reference DNA dilutions and then averaged. Specimens with ratios≧1.2 were considered positive for gain, and specimens with ratios≦0.8 were considered positive for loss.

### Quantitative PCR Validation of Eight Regional Aberrations

Within GISTIC, samples were scored as positive or negative for the presence of the aberration based on the median-normalized log ratio of the peak limit. As confirmation of the selected cut-off log ratio, treatment-naive specimens in DS1 that scored positive for eight of the significant regions were evaluated by QPCR where of the total 91 aberrations found, all were confirmed with the exception of one, and for three others where the aberration detected did not include the gene tested by QPCR (see Supplemental Table I from Houldsworth et al., Leukemia & Lymphoma 55:920-928 (2014)). The copy number assays used in the present disclosure are listed in Table 3.

TABLE 3
Copy Number Assays

| Band | Gene | Copy Number Assay (ABI) |
|---|---|---|
| 8p23.1 | GATA4 | Hs01297945_cn |
| 8p21.3 | TNFRSF10B | Hs00098983_cn |
| 11q22.3 | ATM | Hs02355120_cn |
| 13q14.2 | DLEU2 | Hs03846573_cn |
| 13q14.2 | RB1 | Hs01344097_cn |
| 17p13.1 | TP53 | Hs05506931_cn |
| 2p16.1 | REL | Hs00231626_cn |
| 3q27.3 | BCL6 | Hs02145887_cn |
| 8q24.21 | MYC | Hs01764918_cn |
| 12q15 | MDM2 | Hs00738157_cn |
| 5p15.33 | TERT (Control) | Cat#4403316 |
| 11p12 | RAG2 (Control) | Hs00705088_cn |

### TP53, NOTCH1, and SF3B1 Mutation Analyses

Genomic DNA was submitted to routine bi-directional Sanger sequencing following amplification, using primers and conditions detailed below. For TP53, exons 5-9 were examined, for NOTCH1, an 845-bp fragment in exon 34, and for SF3B1, exons 14-16. Dilution studies revealed a 20-25% sensitivity of detection of heterozygous mutation.

Exons 5-9 in TP53 were analyzed for mutations by PCR amplification of two fragments (exons 5-6, and 7-9) followed by bi-directional Sanger-based sequencing analysis. The PCR primers were as follows:

Forward PCR primer (exons 5-6),
(SEQ ID NO: 1)
5′-GTTTCTTTGCTGCCGTCTTC-3′;
Reverse PCR primer (exons 5-6),
(SEQ ID NO: 2)
5′-TTGCACATCTCATGGGGTTA-3′;
Forward PCR primer (exons 7-9),
(SEQ ID NO: 3)
5′-AAAAGGCCTCCCCTGCTTGC-3′;
and
Reverse PCR primer (exons 7-9),
(SEQ ID NO: 4)
5′-TGTCTTTGAGGCATCACTGC-3′.

In each reaction, 50 ng DNA was amplified using High Fidelity AmpliTaq Gold DNA polymerase (Applied Biosystems, Foster City, Calif.) generating 590-bp (exons 5-6) and 960-bp (exons 7-9) fragments. When a respectively-sized PCR product was not observed, the PCR was repeated with 100 ng DNA. Following purification, the PCR products were bidirectionally sequenced on the ABI 3130 DNA Analyzer (Applied Biosystems) using the respective PCR amplification primers. In addition, the exons 7-9 PCR product (960 bp) was also subjected to sequencing by two additional nested primers:

Forward sequencing primer 2 (exons 7-9),
(SEQ ID NO: 5)
5′-GGGAGTAGATGGAGCCTGGTT-3′
and
Reverse sequencing primer 2 (exons 7-9),
(SEQ ID NO: 6)
5′-GTCCCATTTAAAAAACCAGGCTCCA-3′.

Primer sequences were derived from a previously published study (Puente et al., Nature 475:101-105 (2011)) or designed in the Primer 3 program (found at frodo.wi.mit.edu/primer3/) with filtering using UCSC In-Silico PCR (found at genome.ucsc.edu). After both automated and manual curation, sequences were compared to germline RefSeq sequences (NG_017013.1) using the Mutation Surveyor (Version 4.0.5, SoftGenetics, State College, Pa.). Bidirectionally confirmed variants were considered polymorphic if found in the NCBI SNP database (found at world wide web.ncbi.nlm.nih.gov/snp) or mutations as found in the IARC TP53 mutation database (found at p53.iarc.fr).

For NOTCH1, one PCR product was amplified from 50 ng of genomic DNA (as described above) using the following primers derived from previously published studies (Puente et al., Nature 475:101-105 (2011)):

Forward PCR primer (exon 34),
(SEQ ID NO: 5)
5′-GGGAGTAGATGGAGCCTGGTT-3′
and
Reverse PCR primer (exon 34),
(SEQ ID NO: 6)
5′-GTCCCATTTAAAAAACCAGGCTCCA-3′.

An 854-bp PCR product was generated and subjected to bidirectional Sanger-based sequence analysis as described above using the following sequencing primers designed to permit sequence evaluation of approximately 630 bp region of exon 34 that contains over 99% of NOTCH1 mutations detected in CLL to date (Fabbri et al., J. Exp. Med. 208:1389-1401 (2011); Puente et al., Nature 475:101-105 (2011); Rossi et al., Blood 119:521-529 (2012)):

Forward sequencing primer,
(SEQ ID NO: 7)
5′-GGCATGGTGCCGAACCAATA-3′
and
Reverse sequencing primer,
(SEQ ID NO: 8)
5′-TACTTGAAGGCCTCCGGAAT-3′.

Confirmed sequence variants were identified in comparison to the germline RefSeq sequence (NG_007458.1) and polymorphisms identified by the NCBI SNP database (found at world wide web.ncbi.nlm.nih.gov/snp).

For SF3B1, two PCR products were amplified from 50 ng of genomic DNA (as described above) using the following primers derived from previously published studies (Rossi et al., Blood 118:6904-6908 (2011)):

Forward PCR primer (exon 14),
(SEQ ID NO: 9)
5′-TCTGTTTATGGAATTGATTATGGA-3′;
Reverse PCR primer (exon 14),
(SEQ ID NO: 10)
5′-ACTAAGGAGGCTGAGCAGGA-3′;
Forward PCR primer (exons 15-16),
(SEQ ID NO: 11)
5′-TGCAGTTTGGCTGAATAGTTG-3′;
and
Reverse PCR primer (exons 15-16),
(SEQ ID NO: 12)
5′-CAAATCAAACAGTATTCGTGTAACAT-3′.

Two PCR products were generated, 478 bp (exon 14) and 609 bp (exons 15-16), respectively, and subjected to bidirectional Sanger-based sequence analysis as described above using the respective PCR amplification primers with the exception of the following: Reverse sequencing primer, SR (exon 14), 5′-CAACTTACCATGTTCAATGATTTC-3′ (SEQ ID NO: 13).

Confirmed sequence variants were identified in comparison to the germline RefSeq sequence (NG_032903.1) and polymorphisms identified by the NCBI SNP database (found at world wide web.ncbi.nlm.nih.gov/snp).

### Clinical Correlative Analyses

Pairwise comparisons between biomarkers were tested according to the Fisher's exact test. For univariate associations between biomarkers and time from diagnosis to first treatment (TTFT) or OS from diagnosis, the Kaplan-Meier method and the log-rank statistic were used. Hazard ratios were calculated using Cox regression. A multivariate Cox regression model was fit using stepwise regression methods. A p-value less than 0.05 was considered significant

### CLL Patient Datasets

Table 4 lists the characteristics of the 228 unselected treatment-naïve CLL patients in both datasets used in the present disclosure. Since DS1 was more mature, with a longer median follow-up than DS2, some analyses were independently performed on each dataset. A marginally higher relative proportion of specimens with mutated to unmutated IGHV clonal rearrangements was evident in DS2 than in DS1 (61.9% versus 53.1%), but as expected those with unmutated IGHV significantly exhibited a shorter TTFT and OS in both datasets (p<0.001). An additional 60 specimens sampled from treated CLL patients were also used (38 for DS1, 22 for DS2). Across all specimens, FISH findings for the four commonly detected aberrations were available for 103 specimens (Table 4; see also Supplemental Table I from Houldsworth et al., Leukemia & Lymphoma 55:920-928 (2014)). Of these, 87 were from treatment-naive patients, where del(17p) significantly correlated with shorter OS (p=0.004) and del(11q) exhibited a trend with shorter OS (p=0.086). These specimens were dichotomized with respect to del(17p) and/or del(11q) versus del(13q), +12 or normal, and the former group were confirmed to exhibit significantly shorter OS (p=0.005), but no significant association was found with TTFT (p=0.14).

TABLE 4
Patient Characteristics of CLL Datasets 1 and 2

| | | Dataset DS1 | | Dataset DS2 | |
|---|---|---|---|---|---|
| Untreated Prior to Sampling (n) | | 81 | | 147 | |
| Rai stage | 0 | 26 | (32.1%) | 74 | (50.3%) |
| | I-II | 42 | (51.9%) | 40 | (27.2%) |
| | III-IV | 6 | (7.4%) | 2 | (1.4%) |
| | NA | 7 | (8.6%) | 31 | (21.1%) |
| IGHV mutation status | Unmutated | 37 | (45.7%) | 51 | (34.7%) |
| | Mutated | 43 | (53.1%) | 91 | (61.9%) |

| | | | |
|---|---|---|---|
| Non-clonal | 0 | 5 | (3.4%) |

| | | | |
|---|---|---|---|
| NA | 1 | (1.2%) | 0 |

| Median Diagnosis to Sampling (months) | | 57.3 | 11.1 |
|---|---|---|---|
| Median Follow-Up (months) | | 147.9 | 64.8 |
| Treatment Events | | 43 | 25 |
| Deaths | | 20 | 20 |
| Treated Prior to Sampling (n) | | 38 | 22 |
| Total (n) | | 119 | 169 |
| FISH Aberration^{†} | Specimens (n) | 23 | 80 |
| | del(11q) | 2 | 6 |
| | +12 | 4 | 10 |
| | del(13q) | 14 | 46 |
| | del(17p) | 4 | 12 |

NA = not available;
FISH = fluorescence in situ hybridization.
^{†}Specimens with FISH on same sampling date as aCGH

### Alterations in the CLL Genome Assessed by Targeted aCGH

A targeted oligonucleotide array was designed for clinical diagnostic implementation to represent regions commonly exhibiting genomic imbalance and/or reported to have prognostic value in mature B-cell neoplasms. CBS followed by GISTIC was applied to all specimens and each dataset separately where a total of 18 significant CNAs were identified (Table 5). As confirmation of the selected cut-off log ratio in GISTIC, treatment-naive specimens in DS1 that scored positive for eight of the significant regions were evaluated by QPCR where, of the total 91 aberrations found, all were confirmed with the exception of one, and for three others where the aberration detected did not include the gene tested by QPCR (see Supplemental Table I from Houldsworth et al., Leukemia & Lymphoma 55:920-928 (2014)). Using the 103 specimens with aberrations present in at least 25% of cells by FISH, the sensitivity of detection of aberrations by aCGH was 93.4% and specificity 98.8% for the 76 abnormal and 321 normal FISH results (see Supplemental Table I from Houldsworth et al., Leukemia & Lymphoma 55:920-928 (2014)).

TABLE 5
Significant Regions of Gain and Loss as Identified by Circular Binary Segmentation (CBS) and
Genomic Identification of Significant Targets in Cancer (GISTIC) (NCBI36, Hg18 Assembly)

| | | | | Dataset 1 | | Dataset 2 | |
|---|---|---|---|---|---|---|---|
| Region Limits | | Peak Limits | | Untreated | Treated | Untreated | Treated |
| Start | End | Start | End | (n = 81) | (n = 38) | (n = 147) | (n = 22) |

Gain

| | | | | | | | | |
|---|---|---|---|---|---|---|---|---|
| 1p36.32 | 1 | 3,554,128 | 1 | 2,986,575 | 2 (2.5%) | 2 (5.3%) | 8 (5.4%) | 0 (0.0%) |
| 2p* | 1 | 95,145,278 | 3,362,198 | 3,928,623 | 6 (7.4%) | 5 (13.2%) | 5 (3.4%) | 3 (13.6%) |
| 3q* | 119,808,812 | 197,580,799 | 166,976,533 | 167,342,698 | 2 (2.5%) | 2 (5.3%) | 4 (2.7%) | 2 (9.1%) |
| 7p22.3 | 1 | 3,133,906 | 1 | 1,658,113 | 5 (6.2%) | 4 (10.5%) | 11 (7.5%) | 3 (13.6%) |
| 8q* | 79,442,457 | 143,369,815 | 119,408,616 | 146,274,826 | 2 (2.5%) | 3 (7.9%) | 6 (4.1%) | 0 (0.0%) |
| 12* | 1 | 128,433,046 | 47,778,659 | 48,150,689 | 12 (14.8%) | 4 (10.5%) | 20 (13.6%) | 1 (4.5%) |
| 17q* | 54,867,627 | 74,428,327 | 54,867,627 | 65,775,886 | 2 (2.5%) | 2 (5.3%) | 1 (0.7%) | 1 (4.5%) |
| 18p11.32 | 1 | 3,269,104 | 1 | 762,750 | 4 (4.9%) | 7 (18.4%) | 3 (2.0%) | 0 (0.0%) |
| 18q* | 48,835,323 | 62,630,655 | 51,295,555 | 60,900,467 | 1 (1.2%) | 1 (2.6%) | 6 (4.1%) | 0 (0.0%) |
| 19 | 1 | 63,553,960 | 1 | 3,852,455 | 2 (2.5%) | 1 (2.6%) | 5 (3.4%) | 0 (0.0%) |

Loss

| | | | | | | | | |
|---|---|---|---|---|---|---|---|---|
| 4p15.1 | 23,583,187 | 33,122,761 | 23,583,187 | 33,122,761 | 2 (2.5%) | 2 (5.3%) | 3 (2.0%) | 2 (9.1%) |
| 6q21 | 108,092,926 | 109,659,755 | 108,092,926 | 109,659,755 | 0 (0.0%) | 2 (5.3%) | 5 (3.4%) | 1 (4.5%) |
| 7p22.3 | 1 | 2,005,438 | 1 | 1,726,980 | 7 (8.6%) | 1 (2.6%) | 6 (4.1%) | 1 (4.5%) |
| 8p* | 1 | 24,851,740 | 10,043,135 | 10,523,548 | 3 (3.7%) | 2 (5.3%) | 5 (3.4%) | 1 (4.5%) |
| 11q* | 76,932,697 | 126,037,329 | 111,800,666 | 112,203,507 | 10 (12.3%) | 9 (23.7%) | 10 (6.8%) | 3 (13.6%) |
| 13q* | 33,438,491 | 75,377,448 | 49,568,035 | 49,830,378 | 55 (67.9%) | 21 (55.3%) | 71 (48.3%) | 9 (40.9%) |
| 17p* | 1 | 22,593,075 | 1,491,587 | 8,071,136 | 2 (2.5%) | 6 (15.8%) | 7 (4.8%) | 2 (9.1%) |
| 18p* | 1 | 18,044,710 | 2,539,225 | 4,163,324 | 3 (3.7%) | 2 (5.3%) | 6 (4.1%) | 0 (0.0%) |

*When more than half an arm was involved, the respective arm was listed.

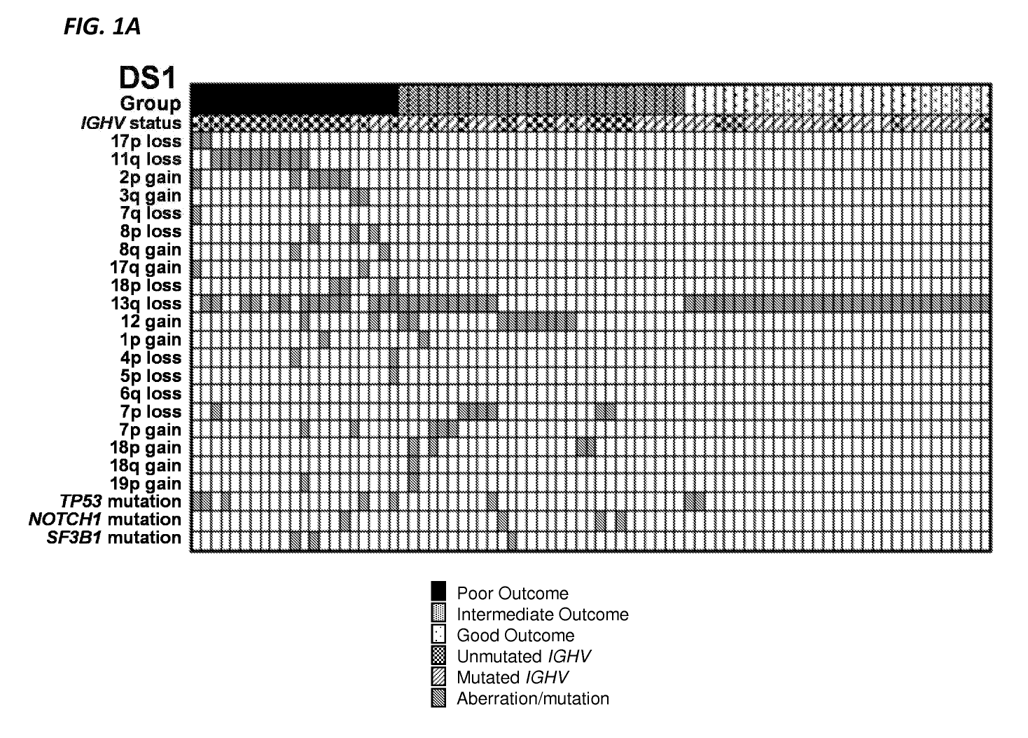

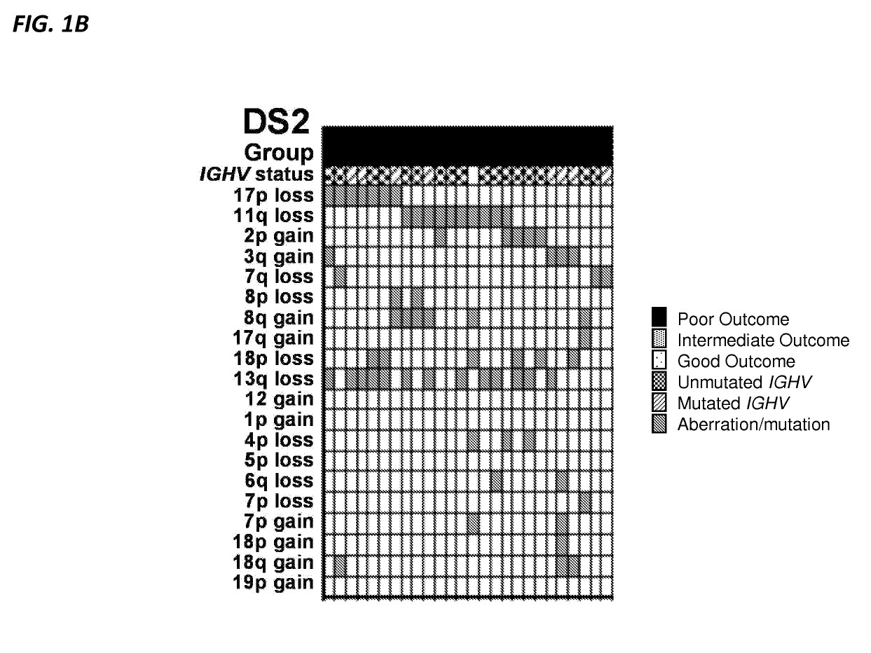

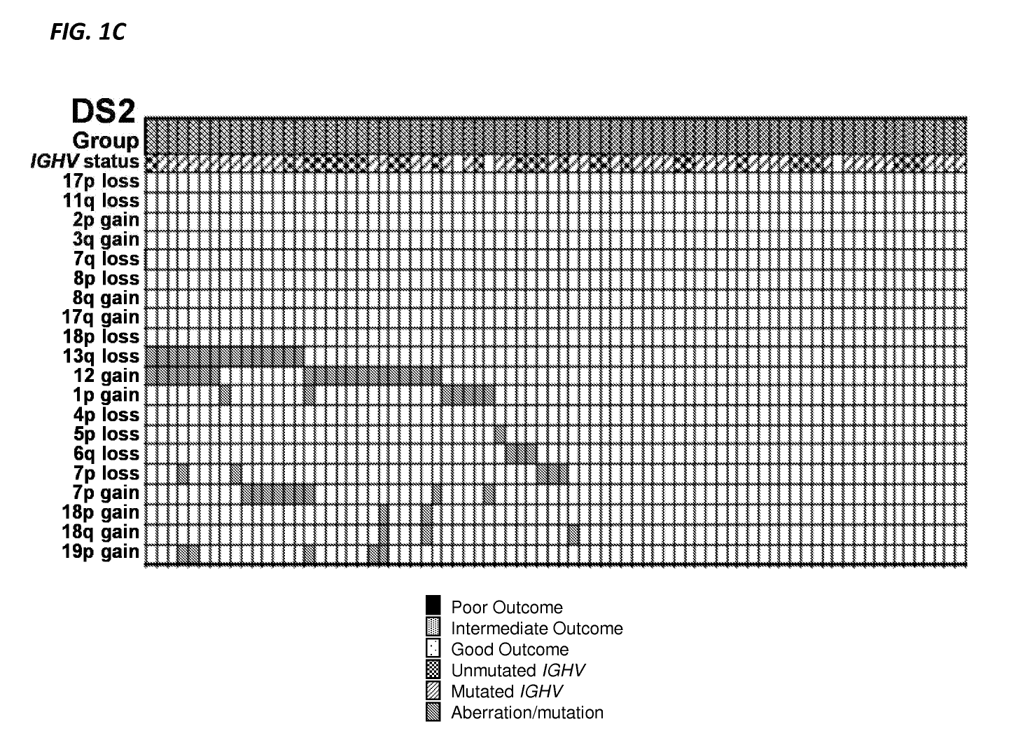

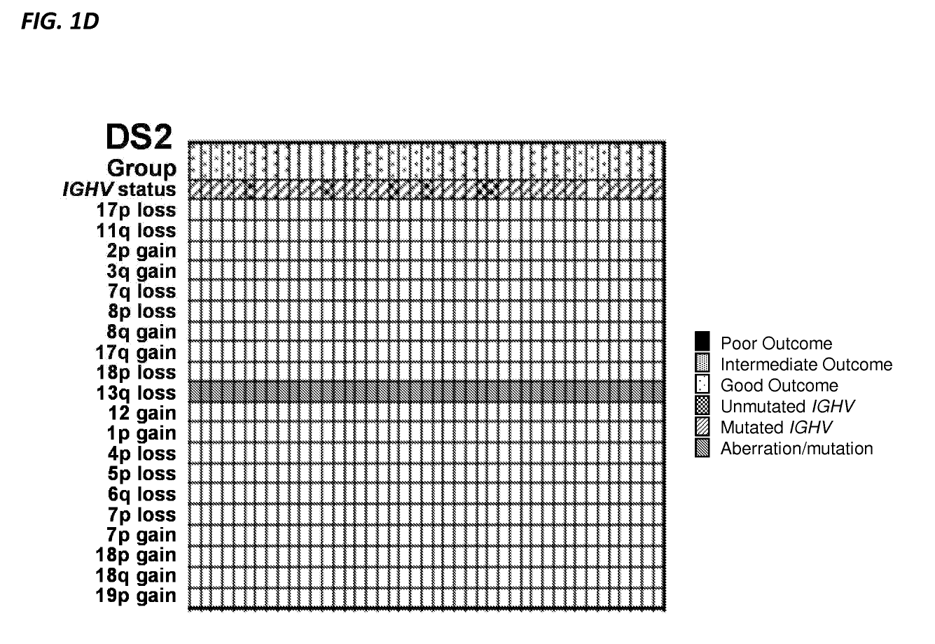

The peak limits in Table 5 provide the most important regions that need to be either gained or lost for the 18 copy number aberrations listed in the table. These are the coordinates used to categorize each sample as positive or negative for each of the 18 copy number aberrations. Considering the 18 significant CNAs, genomic gain/loss was detected in 91.4% and 72.8% of treatment-naive specimens in each respective dataset (FIGS. 1A and B). Since the percentage for DS2 was low, the median-normalized log ratios following CBS of the remaining specimens were individually examined for other aberrations that did not solely comprise backbone probes. Overlapping 7q deletions were found in two specimens and a 5p15 deletion in another. For 7q, a minimally deleted region of chr7:122,471,896-124,803,693 was found across six specimens in both datasets, and for 5p, across two specimens with a minimally deleted region of chr5:5,460,990-8,079,142 (FIGS. 1A and B). Since these aberrations were detected in treatment-naive specimens, they were included in clinical correlative analyses when recurrent within a dataset.

### Genomic Imbalance Associated with Clinical Outcome

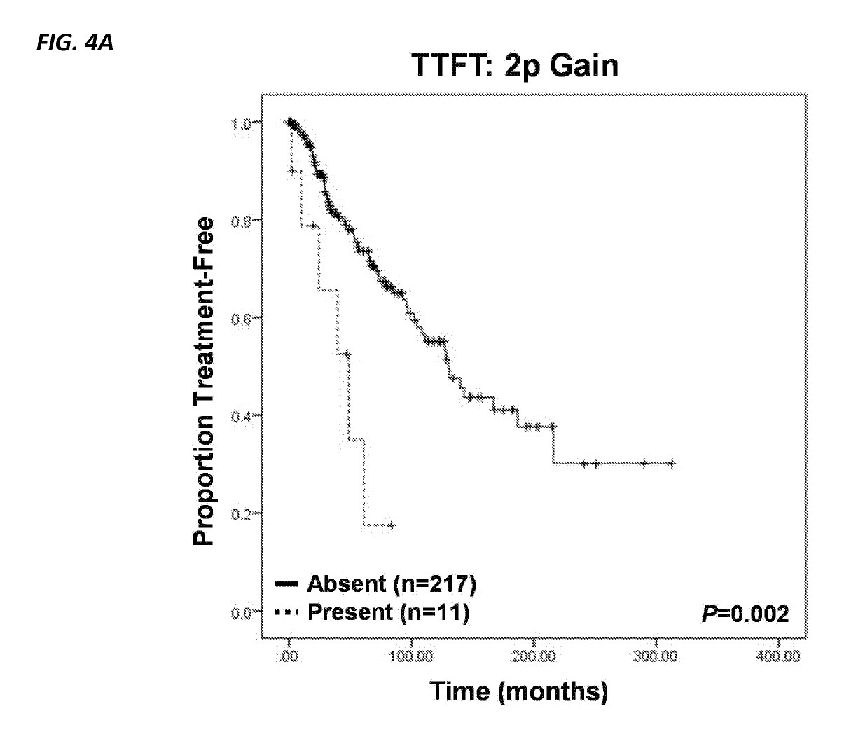

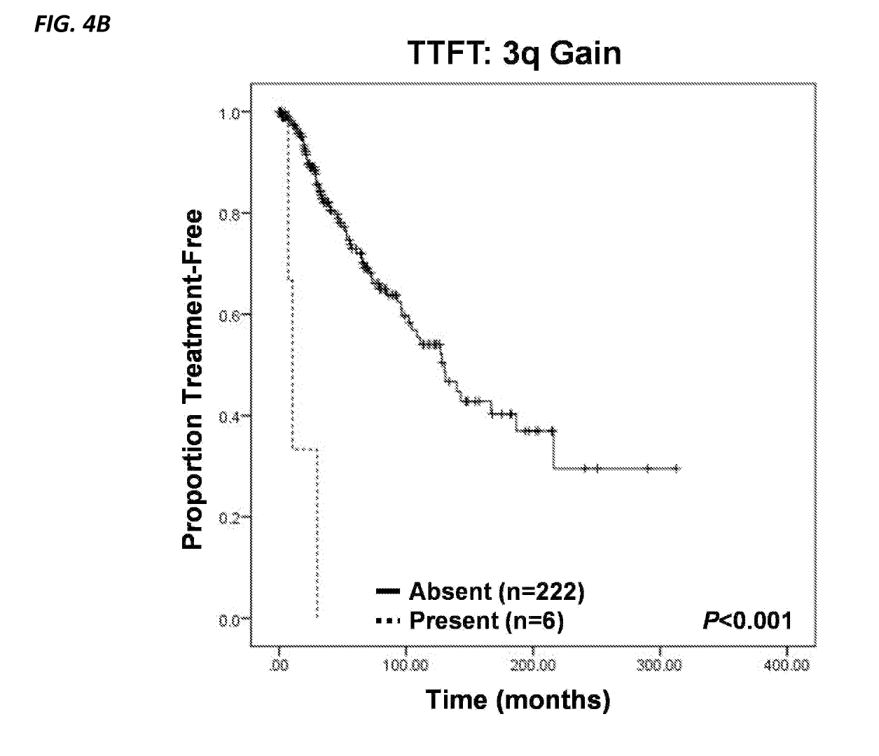

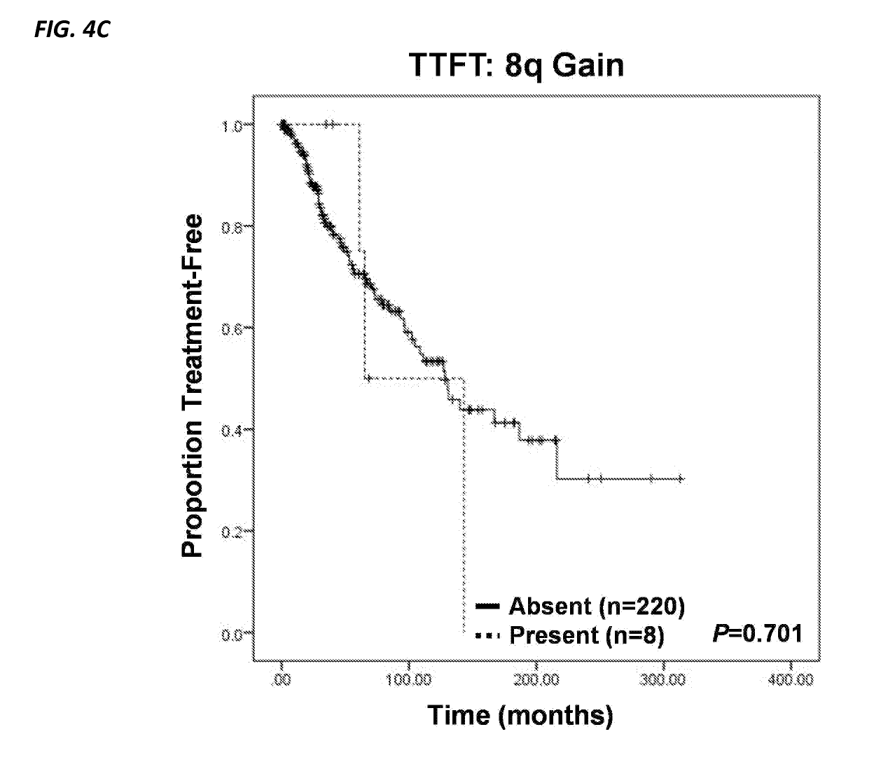

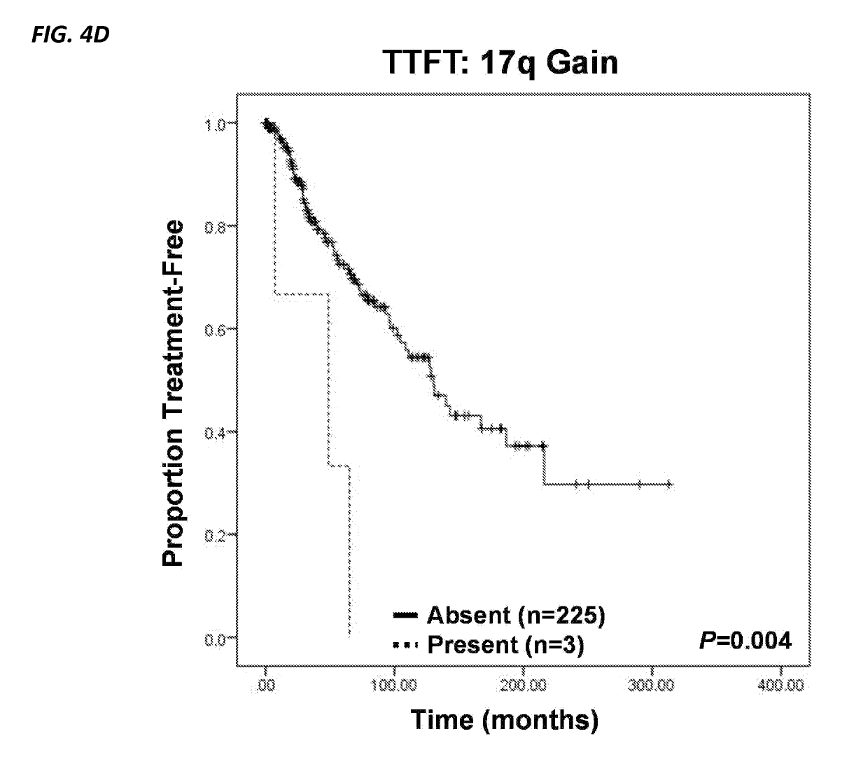

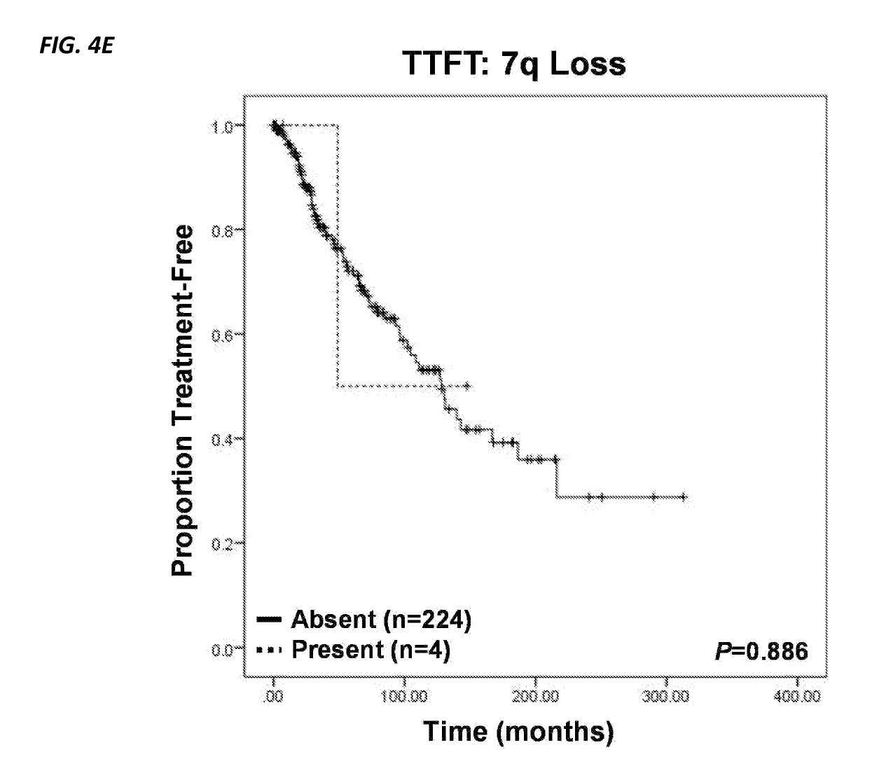

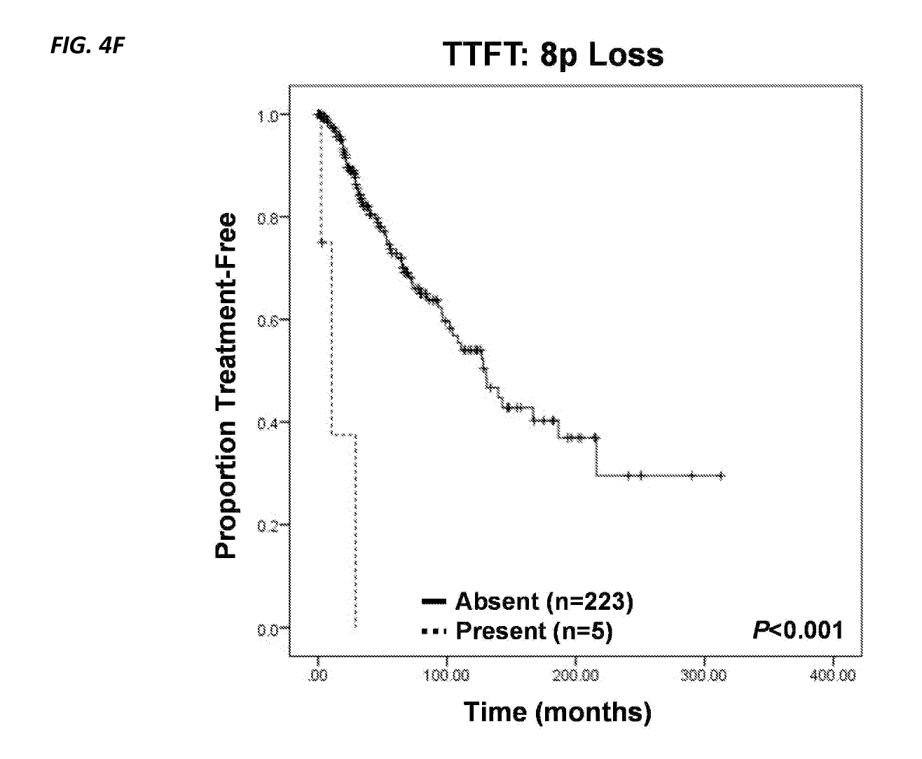

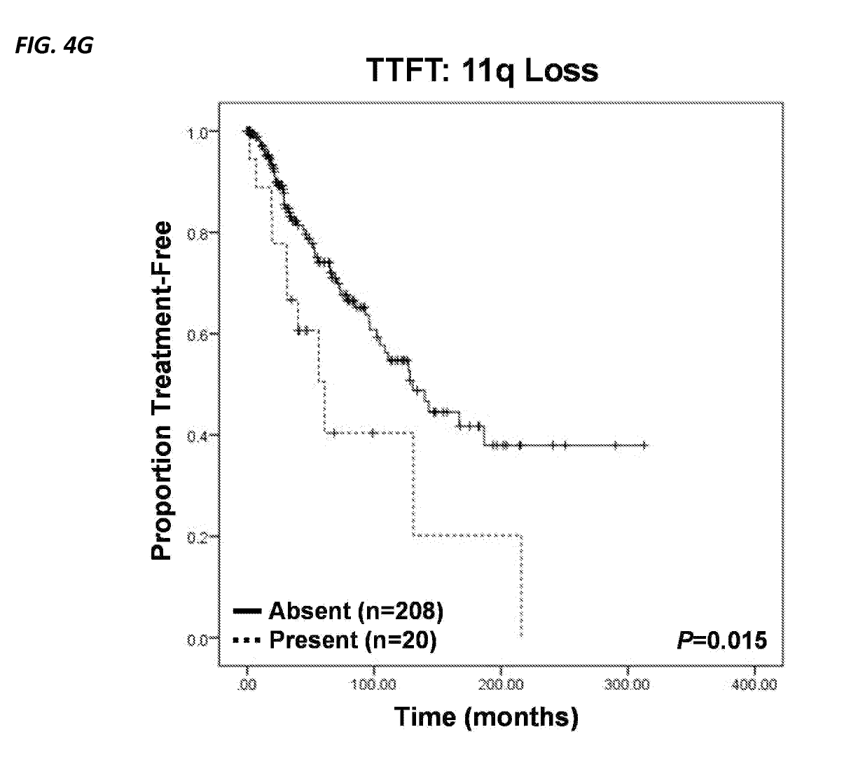

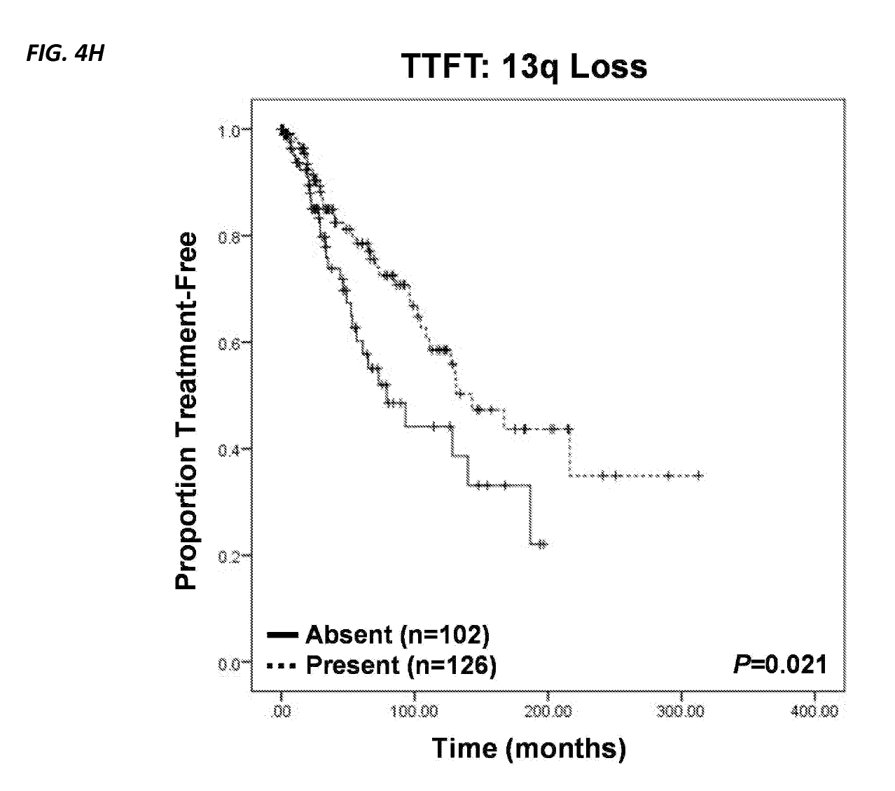

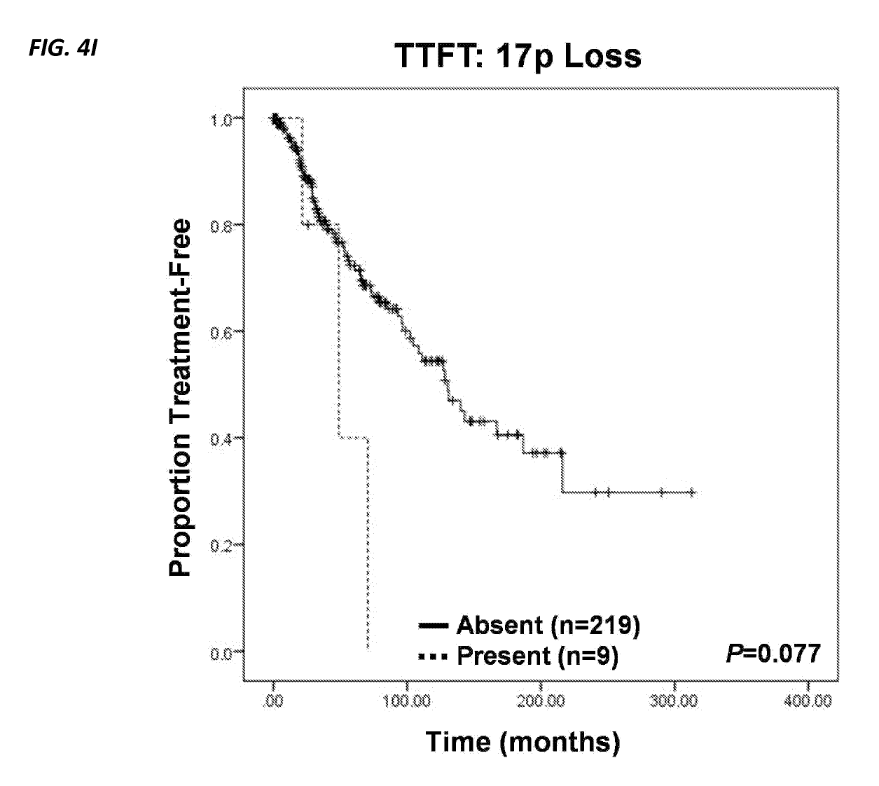

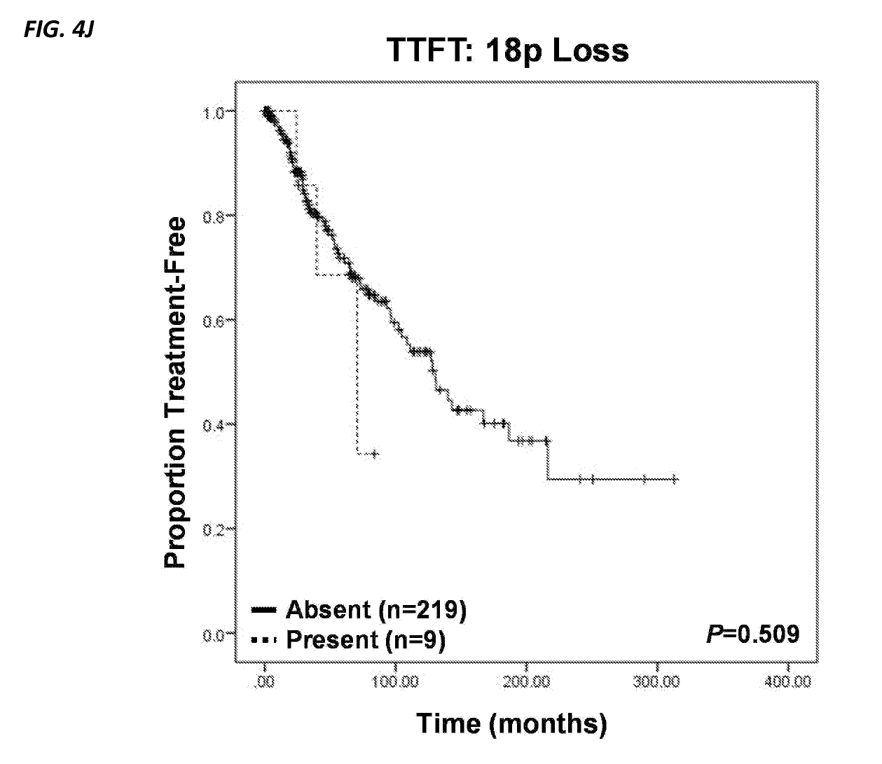

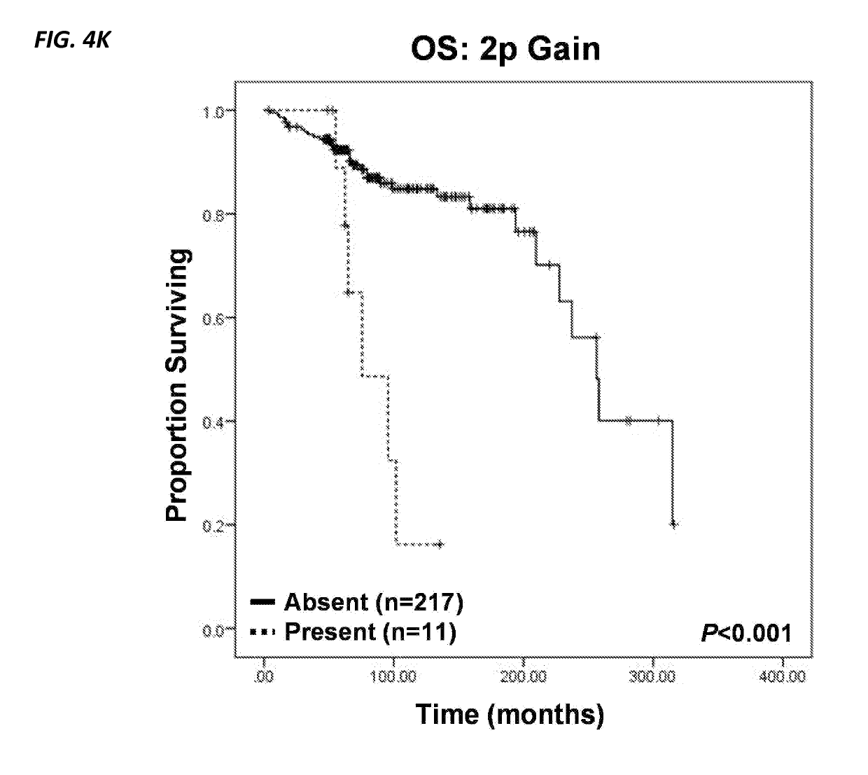

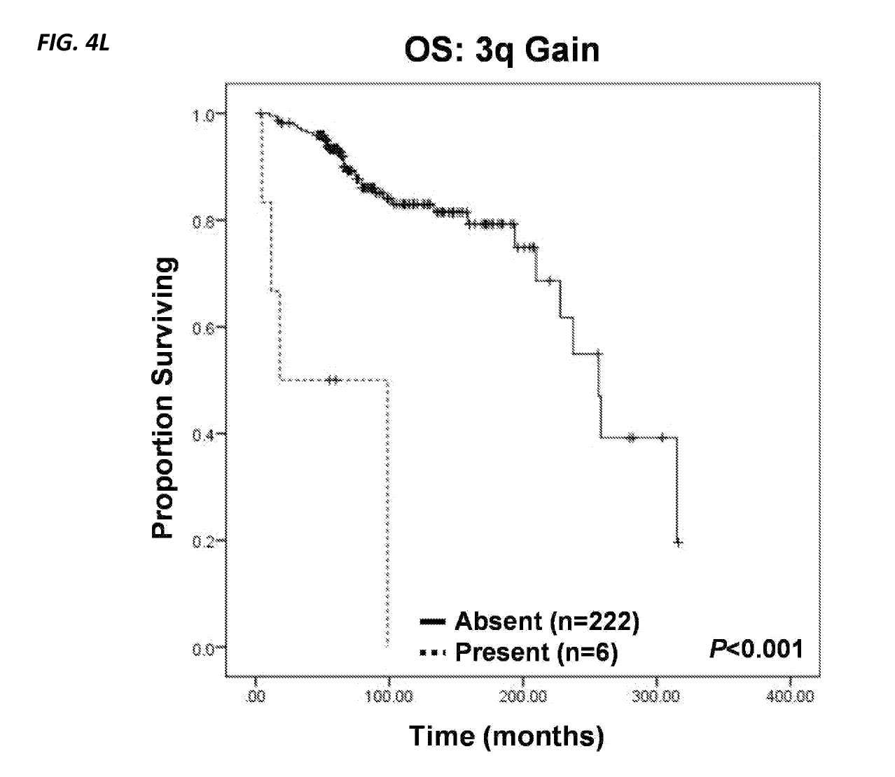

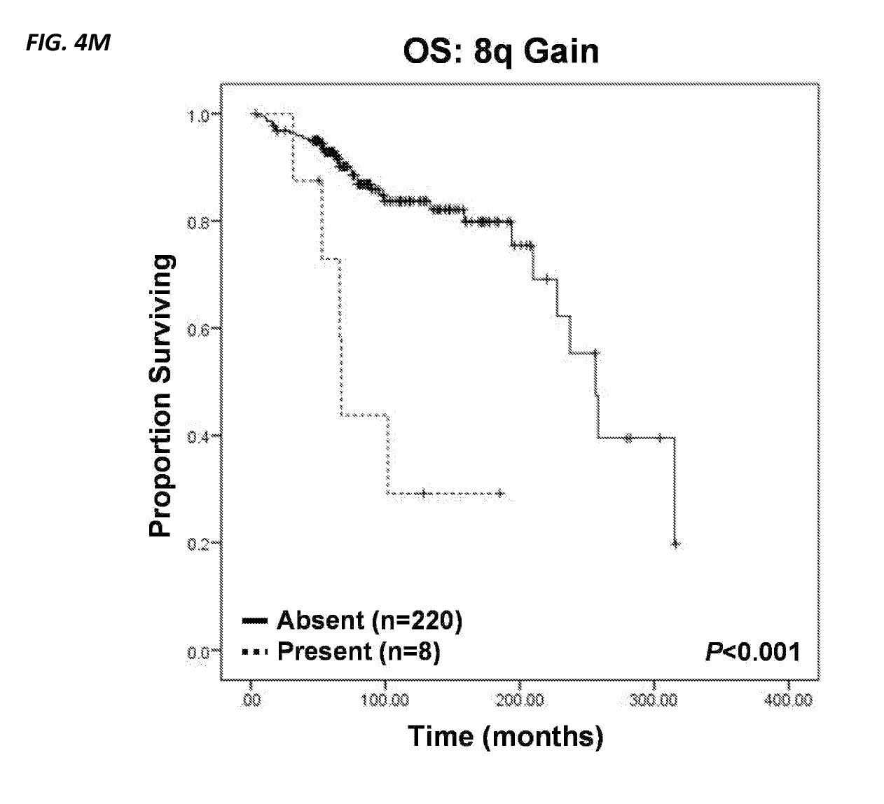

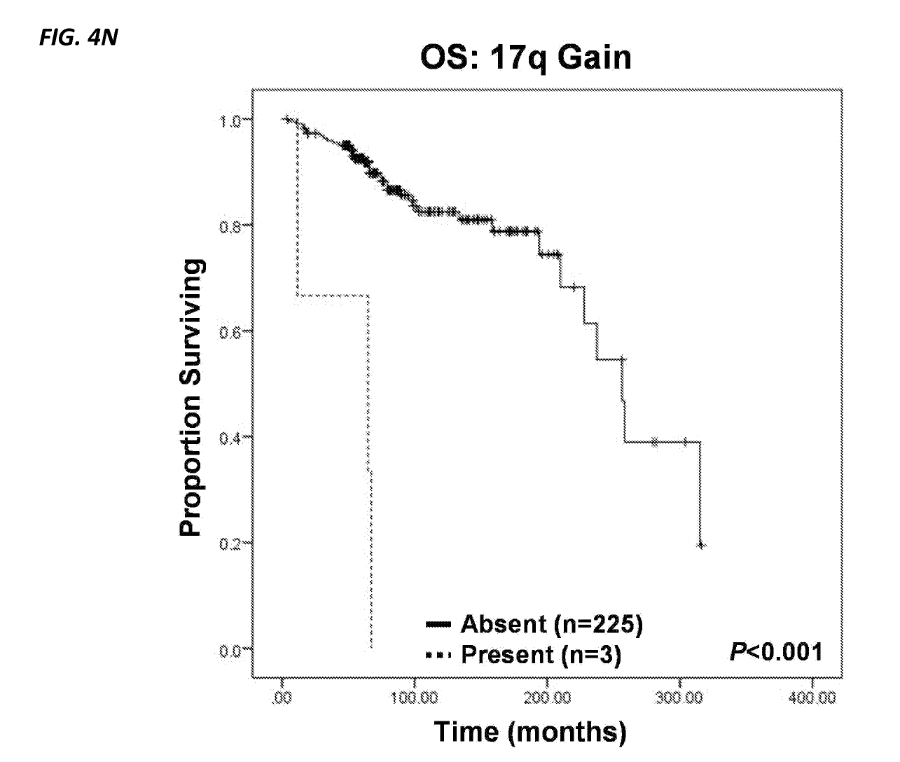

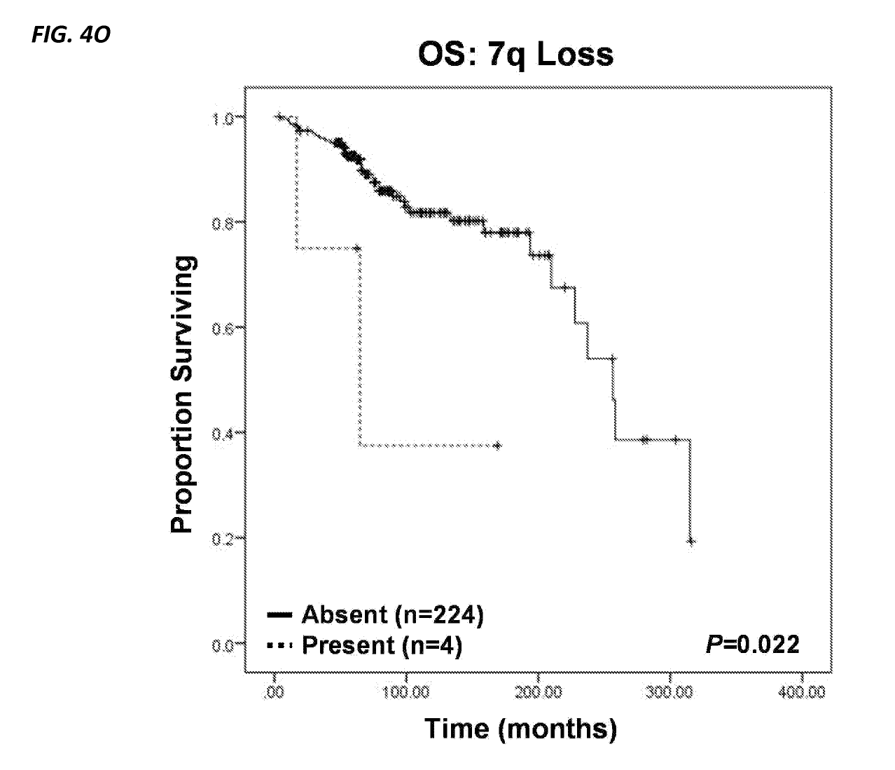

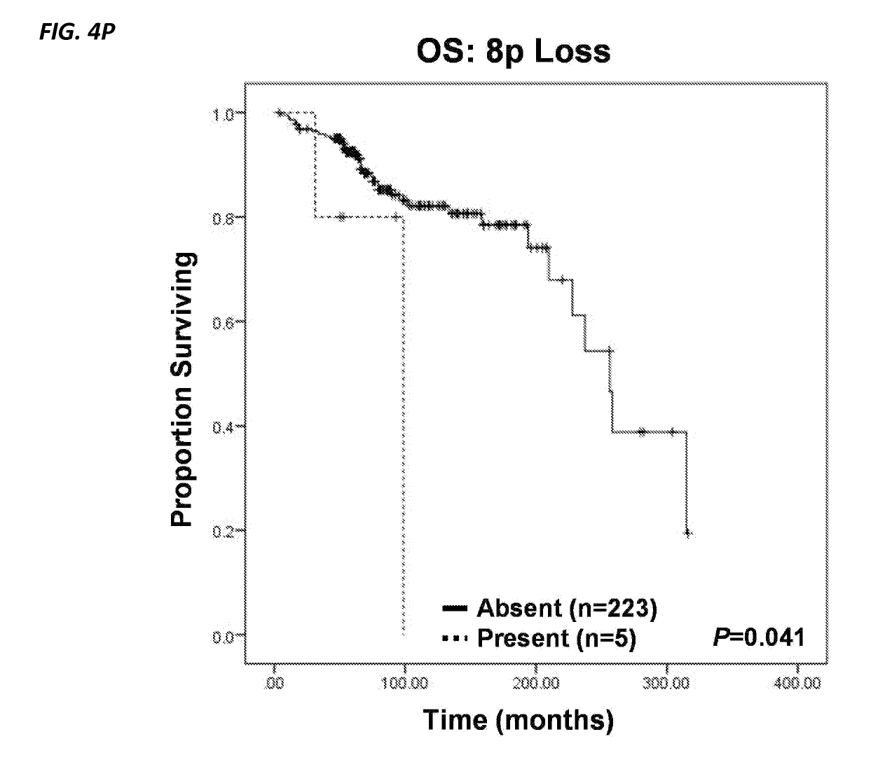

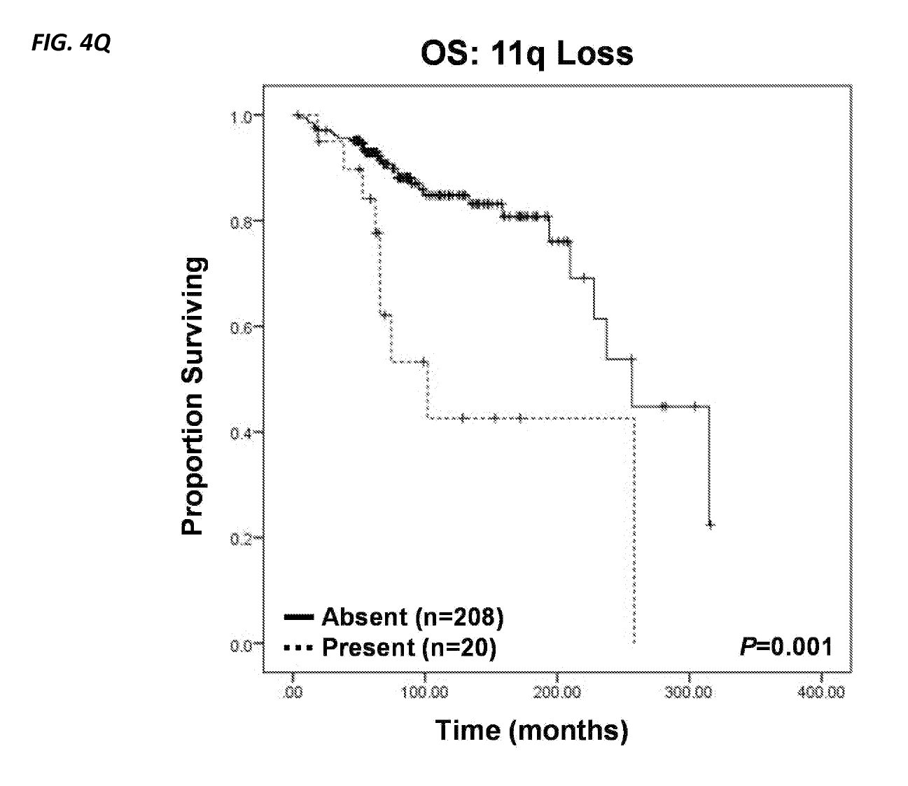

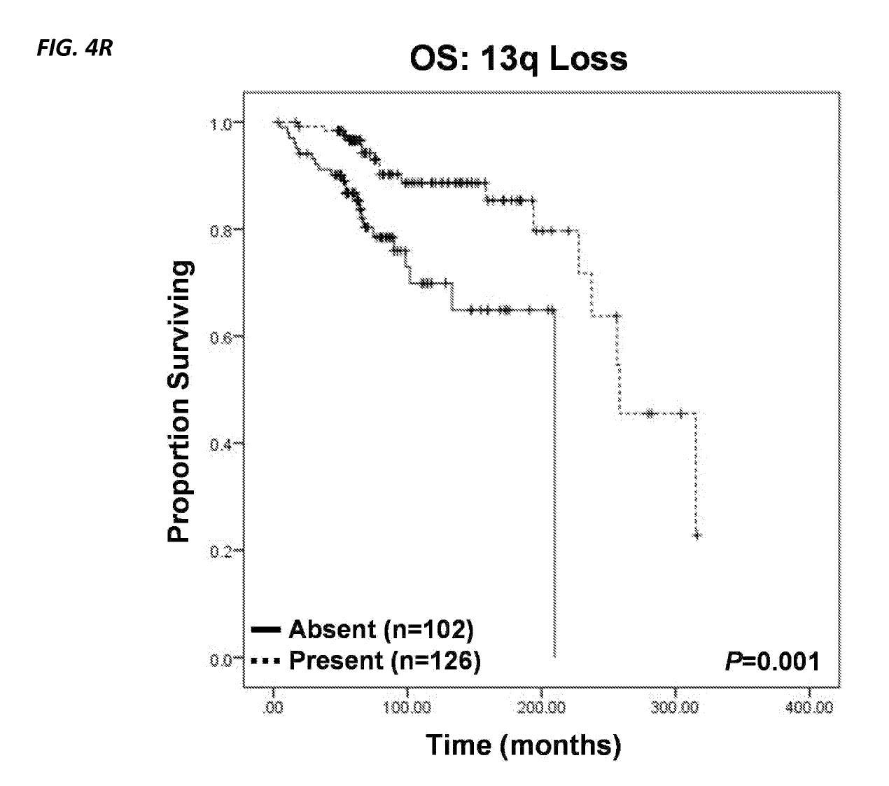

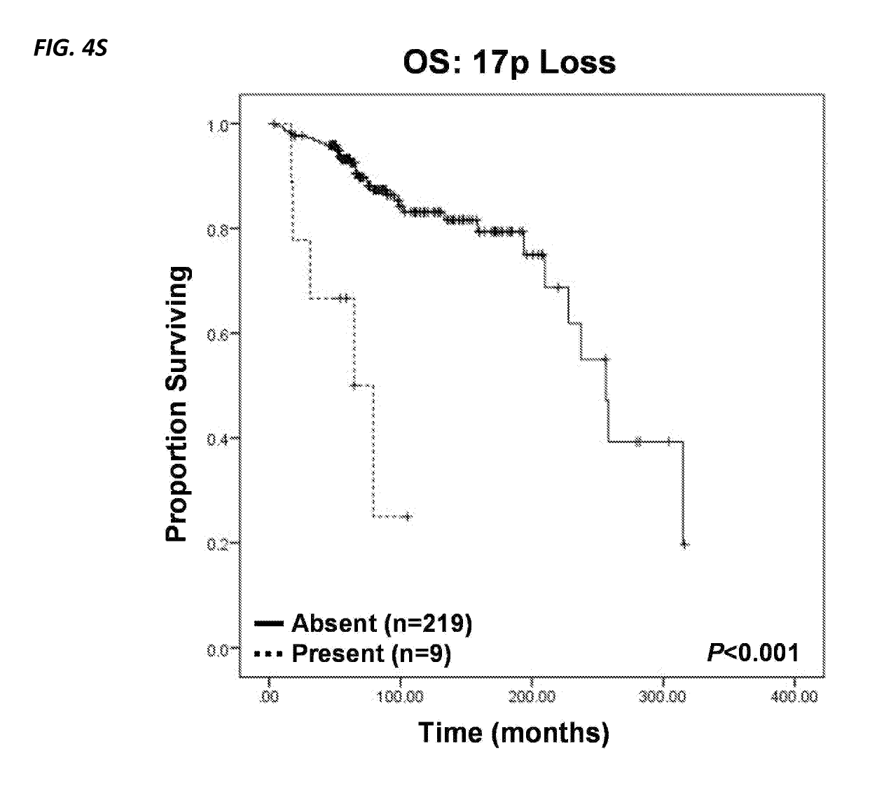

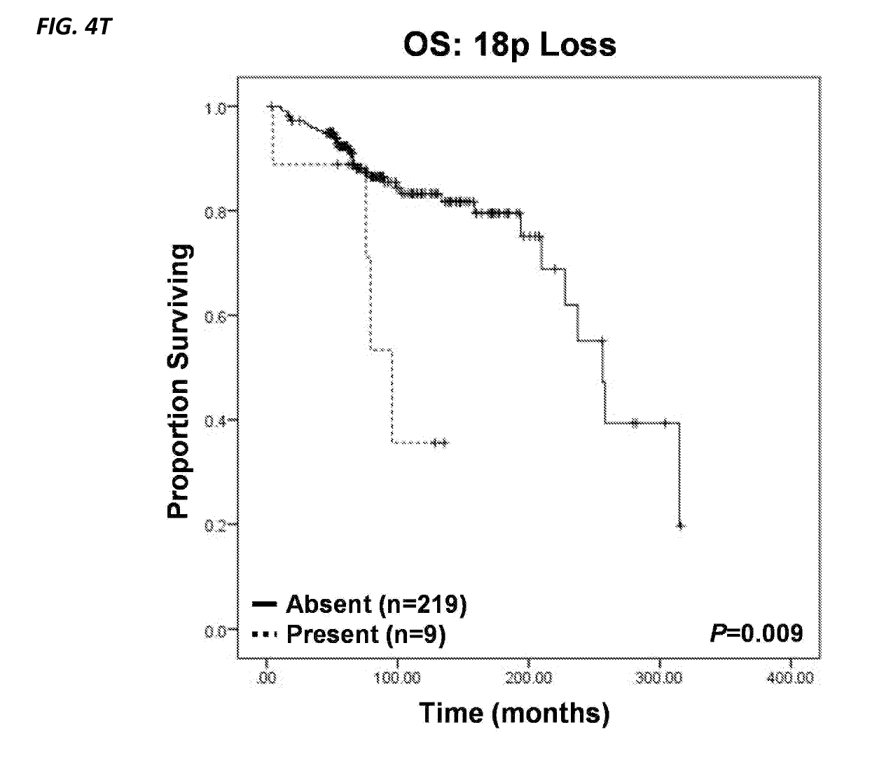

All 20 aberrations (18 from GISTIC plus losses of 5p and 7q) were independently tested for association with clinical endpoints in untreated specimens of each dataset to capture all clinically relevant aberrations. Ten CNAs significantly correlated with TTFT or OS (some with both endpoints), and with the exception of deletion of 13q14, all were associated with shorter times. Table 6 lists the ten CNAs and gives the significance of association with each endpoint for the combined datasets (Kaplan-Meier plots are given for each in FIG. 4A-T). Loss of 18p was significantly associated with 17p loss and 2p gain, as was 7q loss with 17p loss, while 8q gain was associated with 11q loss and 8p loss (Table 7). A multivariate Cox regression analysis incorporating the nine poor prognosis aCGH aberrations and IGHV status, identified 17p loss, 3q and 8q gain, and IGHV status as independent prognostic markers of OS (Table 6). Of note, Rai stage was not entered in the model due to the absence of the information for 38 of the 228 specimens. For TTFT, loss of 8p, gain of 3q, and IGHV mutation status were determined to be independent biomarkers (Table 6).

TABLE 6
Association of Genomic Aberrations with Time from Diagnosis to First Treatment (TTFT) and
Overall Survival (OS) in 228 Treatment-Naïve CLL Specimens

| | | TTFT Endpoint | | OS Endpoint | |
|---|---|---|---|---|---|
| Dataset | | 1 + 2* | p-value (HR [95% CI])^{†} | 1 + 2* | p-value (HR [95% CI])^{†} |
| Gain | chr2: 1-95, 145, 278 | 0.002 | NS | <0.001 | NS |
| | chr3: 119, 808, 812-197, | <0.001 | 0.001 (14.28[2.95-69.24]) | <0.001 | <0.001 (23.08[6.63-80.31)] |
| | 580, 799 | | | | |
| | chr8: 79, 442, 457-143, | 0.701 | NS | 0.001 | 0.001 (6.16[2.32-16.40]) |
| | 369, 815 | | | | |
| | chr17: 54, 867, 627-74, | 0.004 | NS | <0.001 | NS |
| | 428, 327 | | | | |
| Loss | chr7: 122, 471, 896-124, | 0.886 | NS | 0.022 | NS |
| | 803, 693 | | | | |
| | chr8: 1-24, 851, 740 | <0.001 | <0.001 (16.47[3.41-79.52]) | 0.041 | NS |
| | chr11: 76, 932, 697-126, | 0.015 | NS | 0.001 | NS |
| | 037, 329 | | | | |
| | chr13: 33, 438, 491-75, | 0.021 | Not entered | 0.001 | Not entered |
| | 377, 448 | | | | |
| | chr17: 1-22, 593, 075 | <0.001 | NS | <0.001 | 0.023 (3.51[1.20-10.29)] |
| | chr18: 1-18, 044, 710 | 0.509 | NS | 0.009 | NS |
| IGHV Mutation Status (unmutated) | | <0.001 | <0.001 (5.64[3.30-9.64]) | <0.001 | <0.001 (6.57[3.01-14.34]) |
| Dataset | | 1* | | 1* | |
| Mutation^{‡} | TP53 (all) | 0.767 | Not entered | 0.147 | Not entered |
| | TP53 (excluding | 0.001 | Not entered | 0.007 | Not entered |
| | IGHV mut) | | | | |
| | NOTCH1 | 0.021 | Not entered | 0.021 | Not entered |
| | SF3B1 | 0.004 | Not entered | 0.238 | Not entered |

NS = not significant;
not entered = variable not entered in multivariate regression analysis.
*= Univariate p-value from log-rank test, all variables associated with shorter TTFT and/or OS with exception of 13q.
^{†=} Multivariate p-value after variable selection (hazards ratio [95% confidence intervals]).
^{‡=} Only performed for DS1.

TABLE 7
Pairwise Comparisons of Clinically Relevant CNAs
According to Fisher's Exact Test

| | 17p | 11q | 2p | 3q | 7q | 8p | 8q | 17q | 18p | |
|---|---|---|---|---|---|---|---|---|---|---|
| | loss | loss | gain | gain | loss | loss | gain | gain | loss | IGHV |
| 17p loss | * | 1.000 | 0.373 | 0.222 | 0.008 | 0.188 | 0.255 | 0.117 | 0.036 | 0.160 |
| 11q loss | * | * | 0.057 | 1.000 | 1.000 | 0.363 | 0.001 | 1.000 | 1.000 | <0.001 |
| 2p gain | * | * | * | 1.000 | 0.185 | 0.226 | 0.303 | <0.001 | <0.001 | <0.001 |
| 3q gain | * | * | * | * | 1.000 | 0.129 | 1.000 | 0.200 | 0.200 | 1.000 |
| 7q loss | * | * | * | * | * | 1.000 | 1.000 | 1.000 | 1.000 | 0.303 |
| 8p loss | * | * | * | * | * | * | 0.008 | 1.000 | 1.000 | 1.000 |
| 8q gain | * | * | * | * | * | * | * | 1.000 | 1.000 | 0.439 |
| 17q gain | * | * | * | * | * | * | * | * | 1.000 | 0.061 |
| 18p loss | * | * | * | * | * | * | * | * | * | 0.007 |
| IGHV | * | * | * | * | * | * | * | * | * | * |

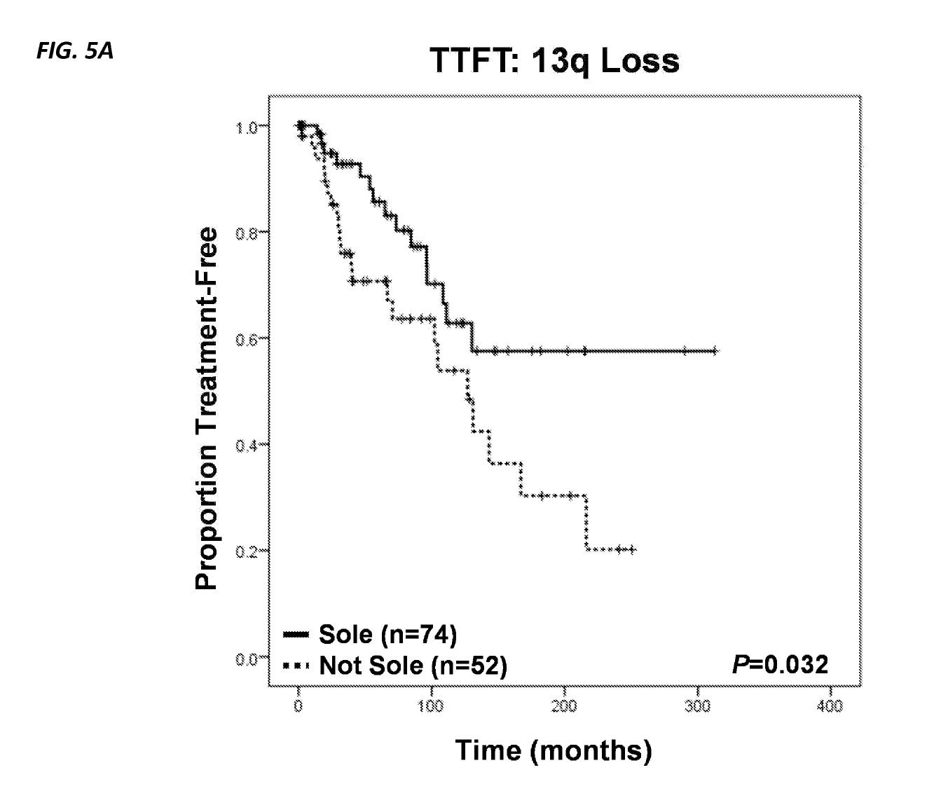

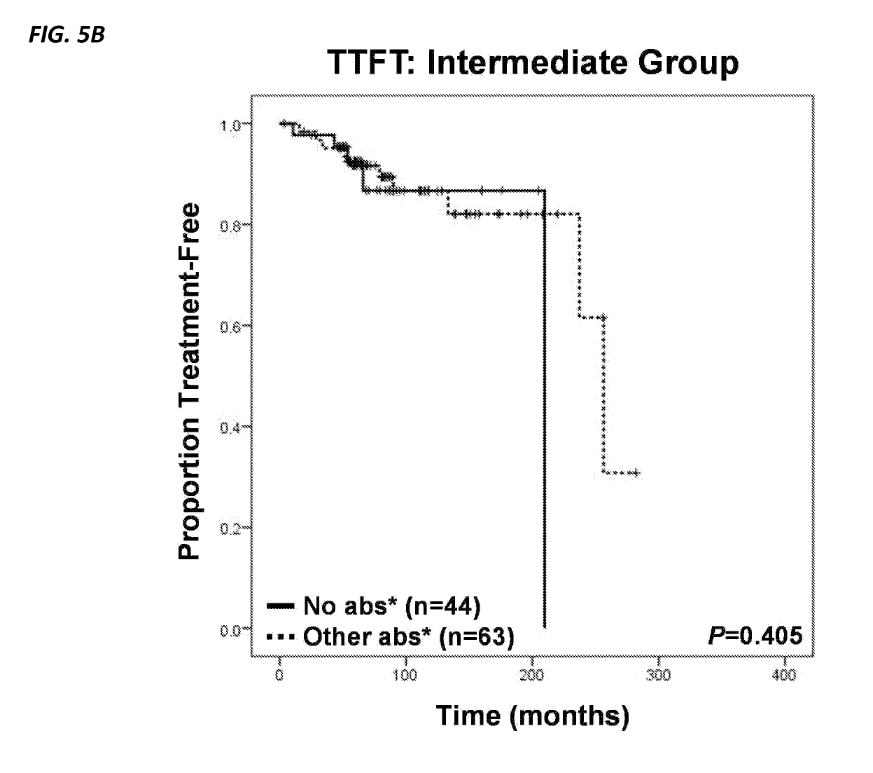

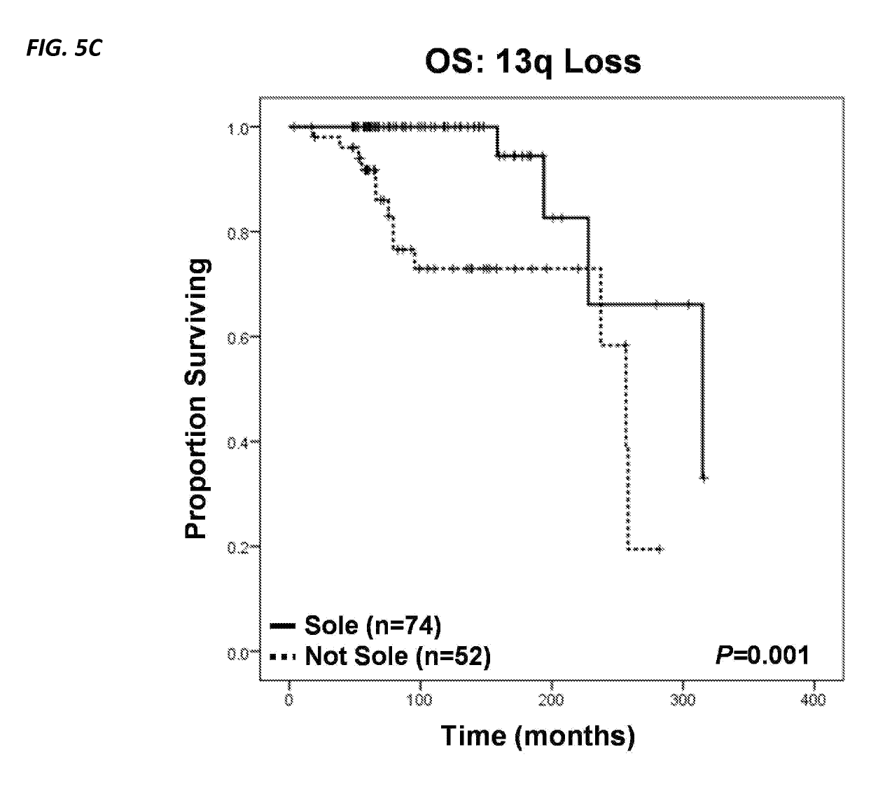

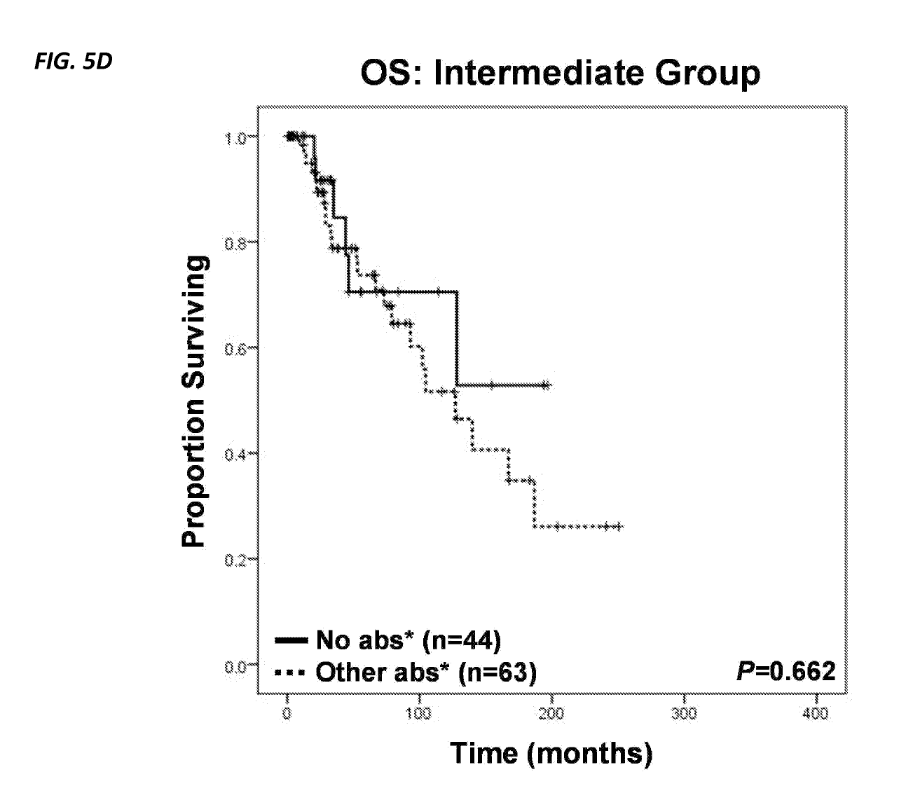

As expected, loss of 17p and 11q were amongst the nine aCGH markers univariately associated with adverse outcome. These aberrations were found in 29 treatment-naïve specimens (12.7%) across both datasets. Importantly, an additional 18 specimens (7.9%) bore at least one of the other seven poor aCGH markers: gain of 2p, 3q, 8q, or 17q, or loss of 7q, 8p, or 18p. Combined, these 47 specimens were grouped as having poor prognosis. In a hierarchical manner somewhat analogous to the previous stratification scheme based on aberrations detected by FISH (Dohner et al., N. Engl. J. Med. 343:1910-1916 (2000)), a second non-overlapping group of 74 specimens were identified that had 13q14 deletions but no additional aberrations at the ten other recurrent loci (gain: 1p, 7p, 12, 18p, 18q, 19, loss: 4p, 5p, 6q, 7p). The respective patients with 13q14 loss as a sole abnormality were grouped as having a good prognosis, as they exhibited a highly favorable outcome when compared with those with 13q14 deletions plus other aberrations (FIGS. 5A and C). Lastly, a third group comprised two subsets: 63 that only exhibited any of the ten recurrent loci used to define 13q14 loss as a sole abnormality (gain: 1p, 7p, 12, 18p, 18q, 19, loss: 4p, 5p, 6q, 7p) and 44 that did not carry any of the total 20 aberrations. Since no difference in TTFT or OS was found between these two subsets (p=0.405, 0.662, respectively), they were joined into one group (107 specimens) with an intermediate prognosis (FIGS. 5B and D).

Thus, all treatment naïve specimens in DS1 and DS2 were classified into one of three prognostic groups based on the presence/absence of the 20 CNAs (FIG. 1; see also Supplemental Table I from Houldsworth et al., Leukemia & Lymphoma 55:920-928 (2014)). In FIGS. 1A and B, each treatment-naive specimen in DS1 (n=81) and DS2 (n=147), respectively, is represented as a column. The first row of each provides the prognostic classification group according to the presence/absence of the aCGH aberrations recorded in the rows below (total of 155 losses and 109 gains). The mutation status of the IGHV clonal rearrangement in both datasets, and the presence/absence of TP53, NOTCH1, and SF3B1 mutations in DS1 are also shown. Full CNA and mutation data for each specimen are provided in Houldsworth et al., Leukemia & Lymphoma 55:920-928 (2014), herein incorporated by reference in its entirety for all purposes.

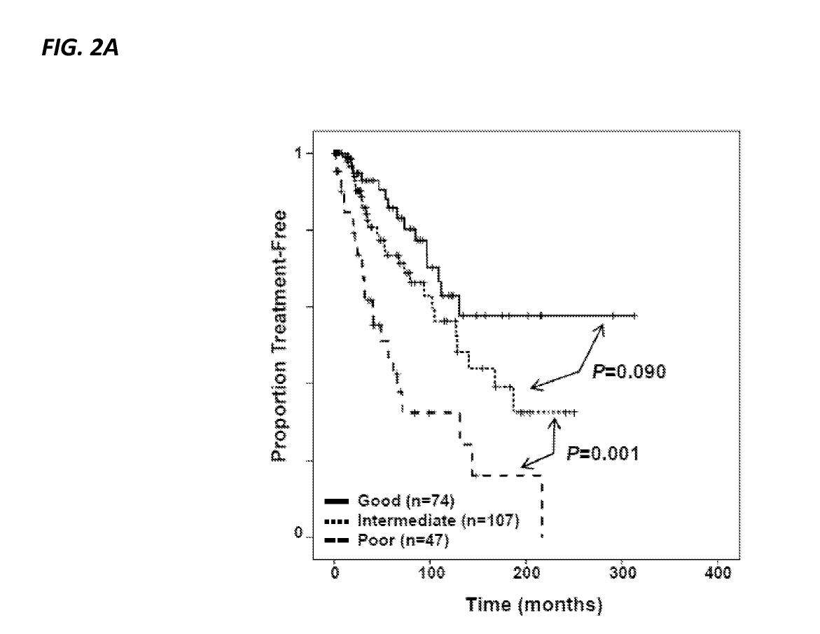

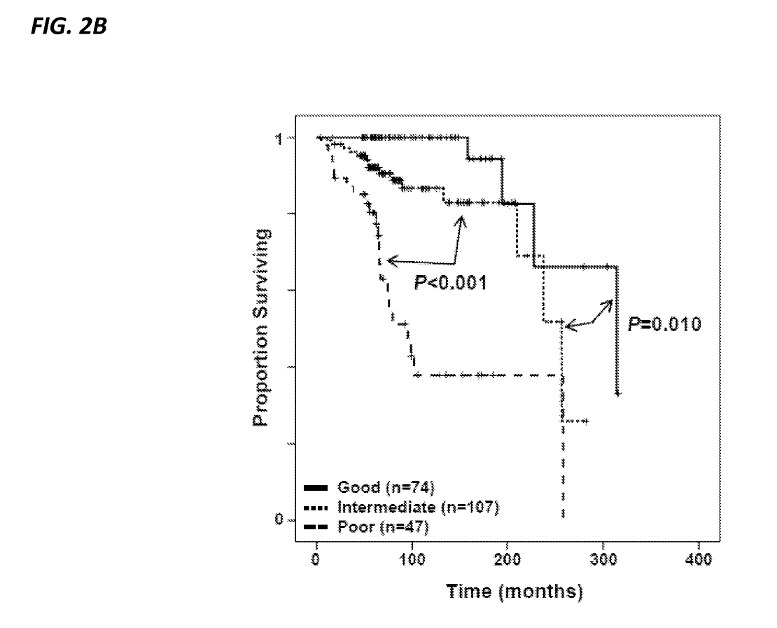

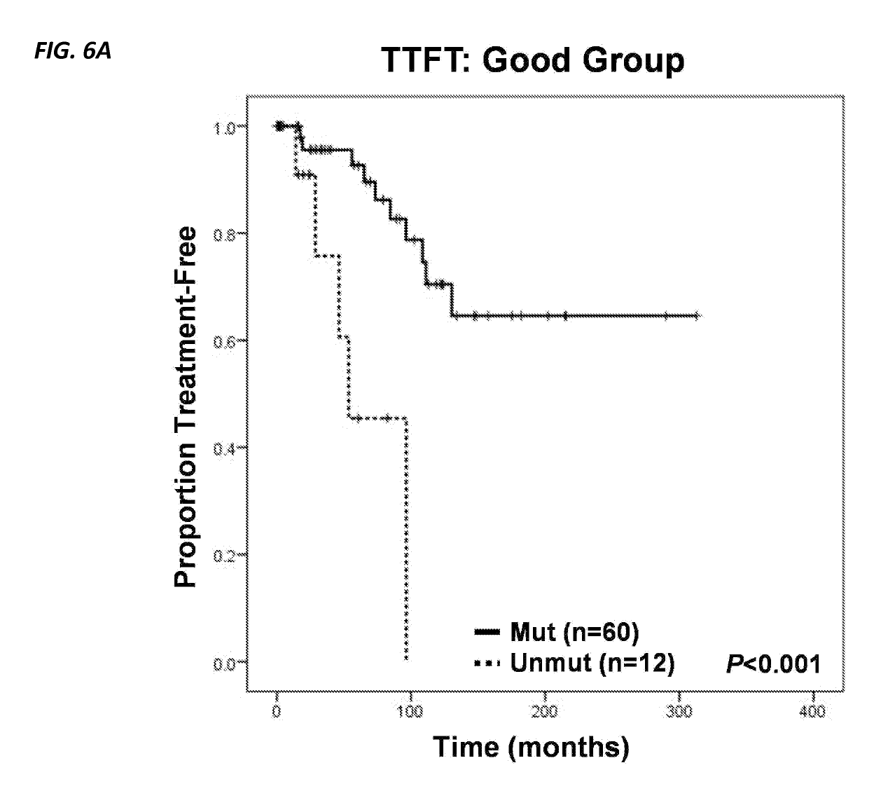

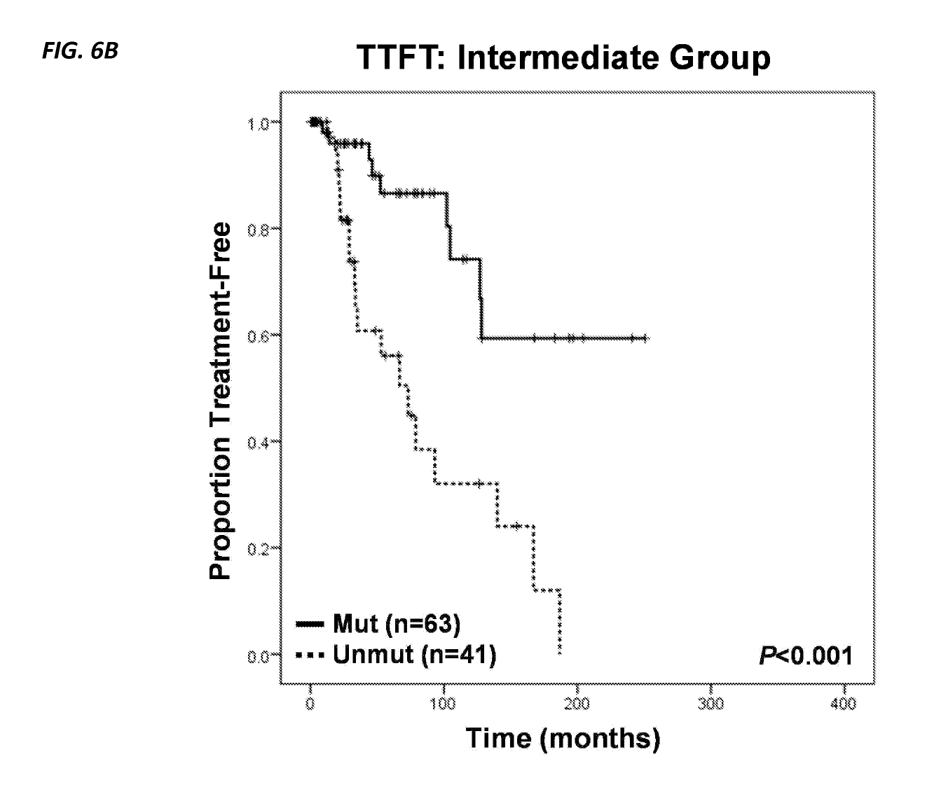

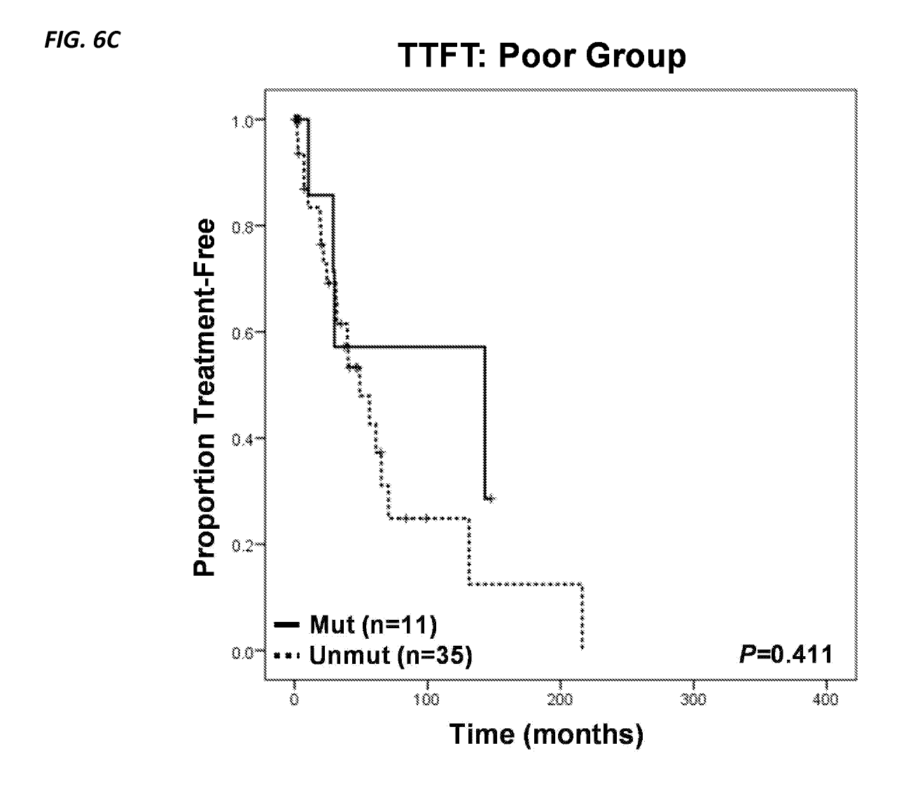

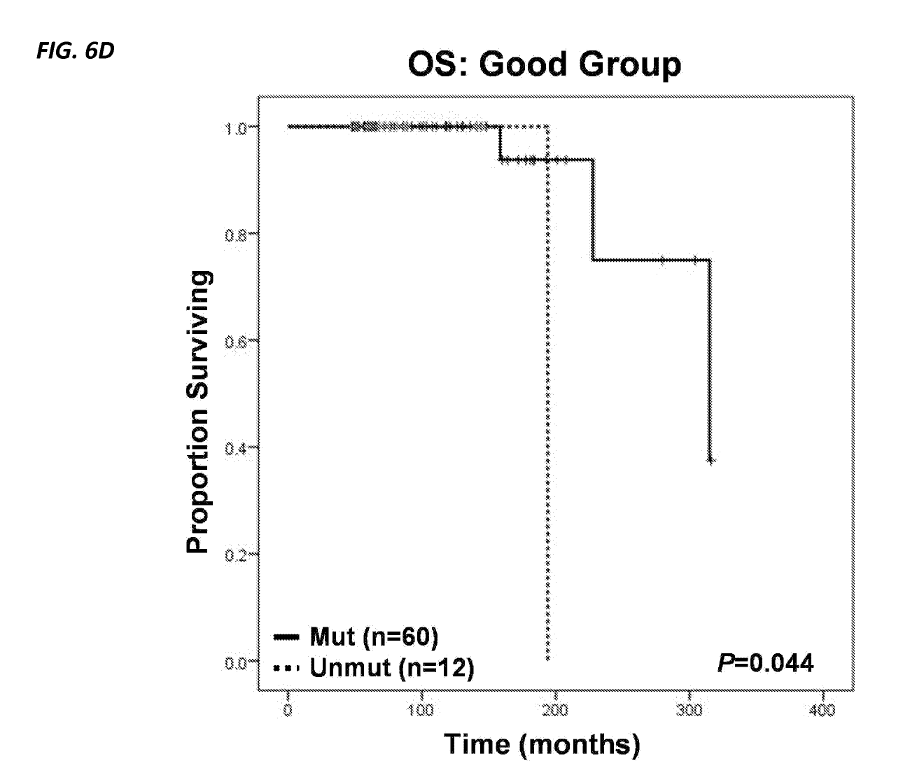

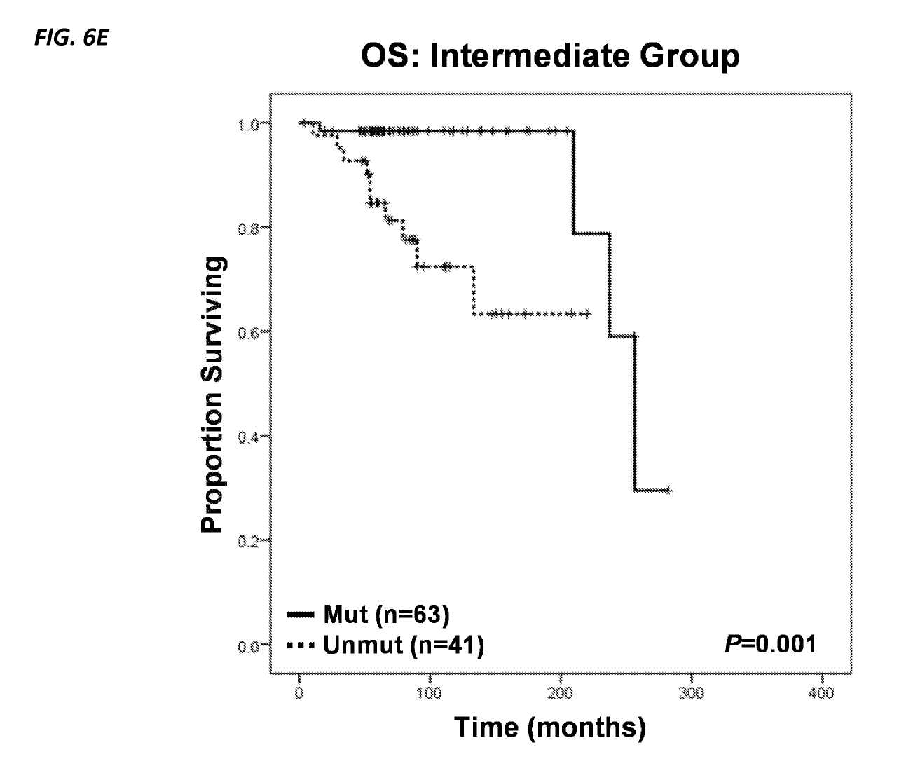

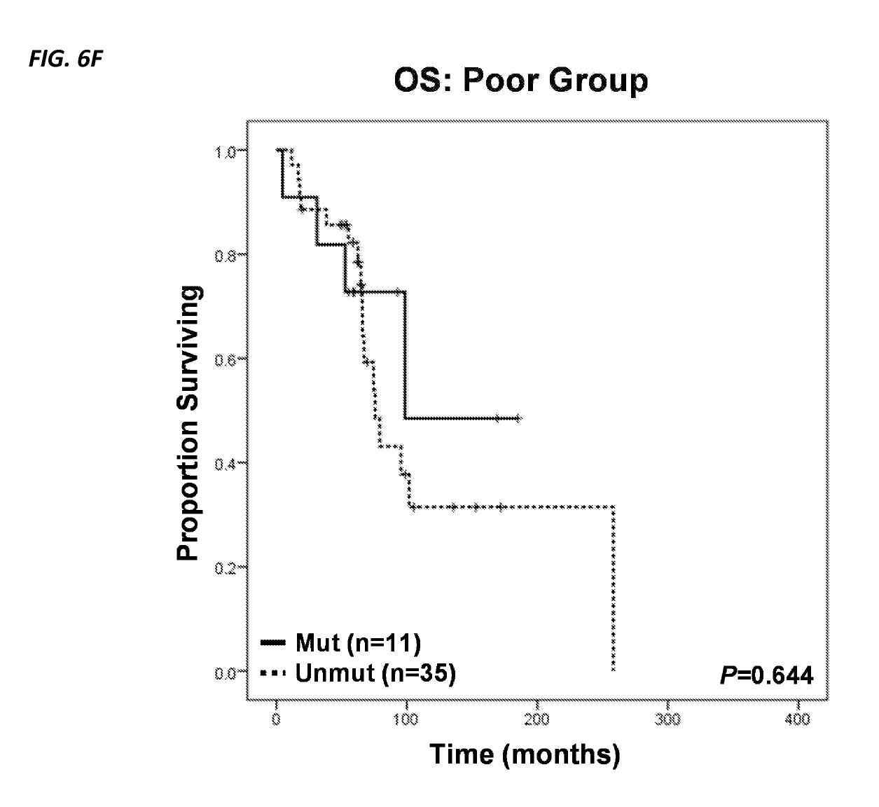

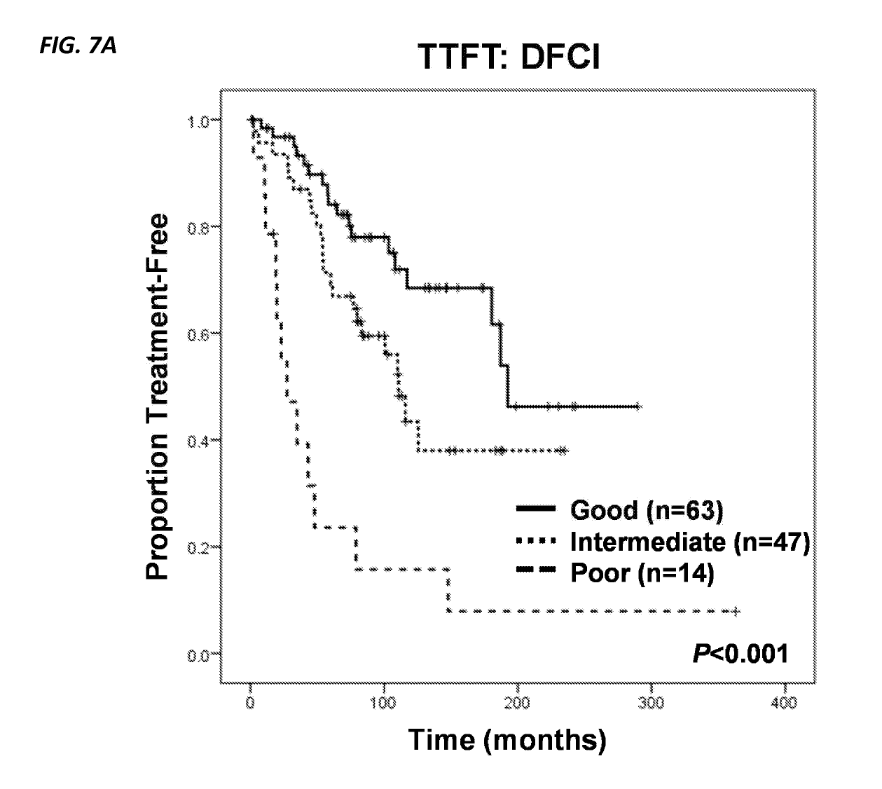

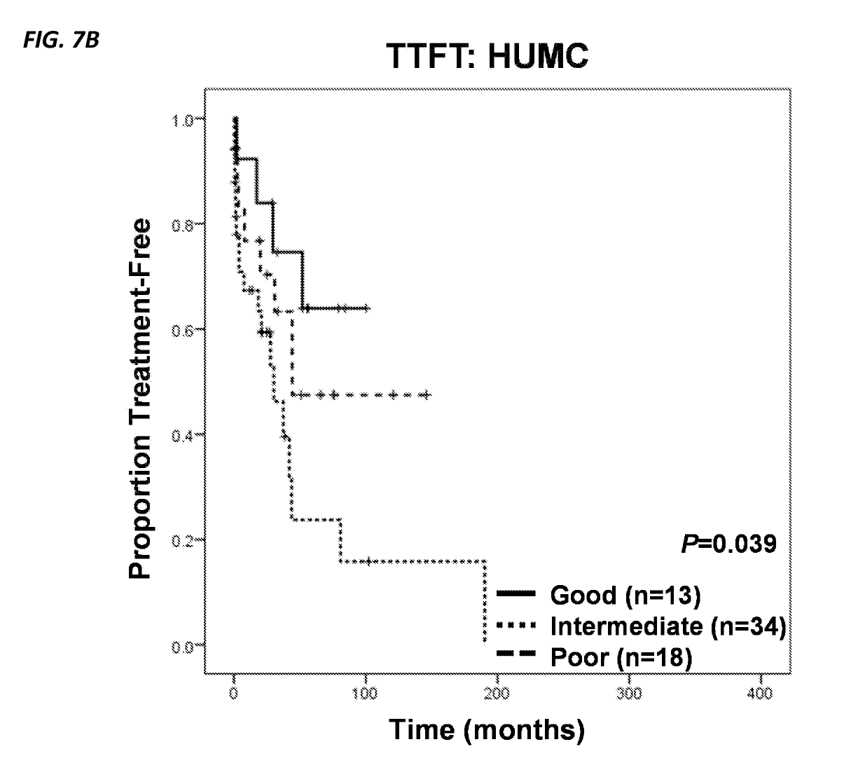

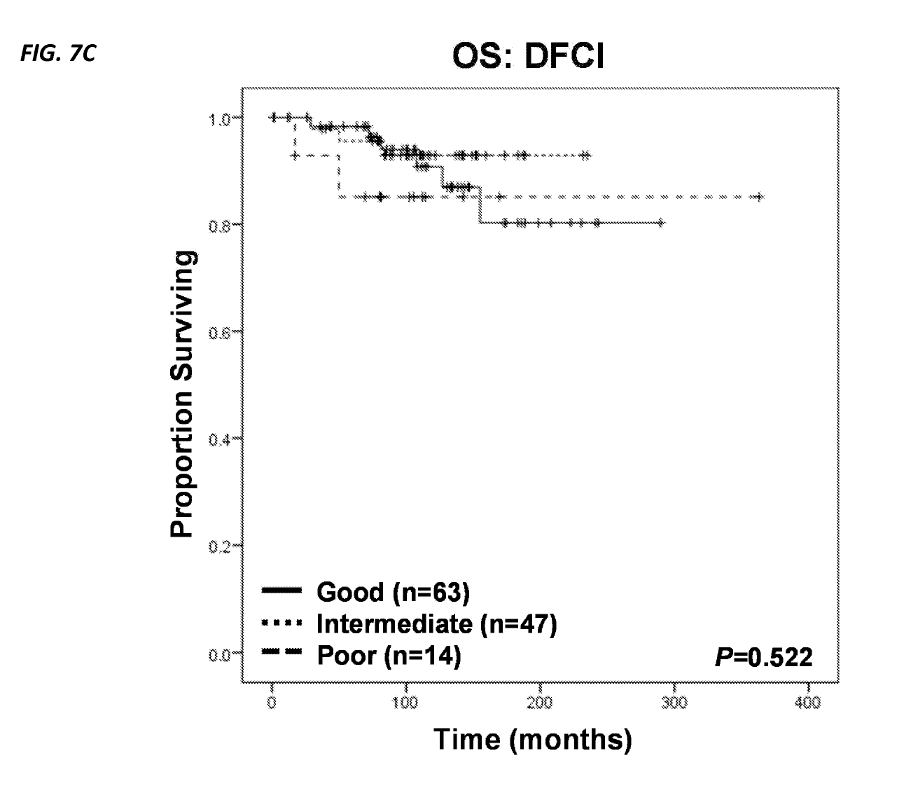

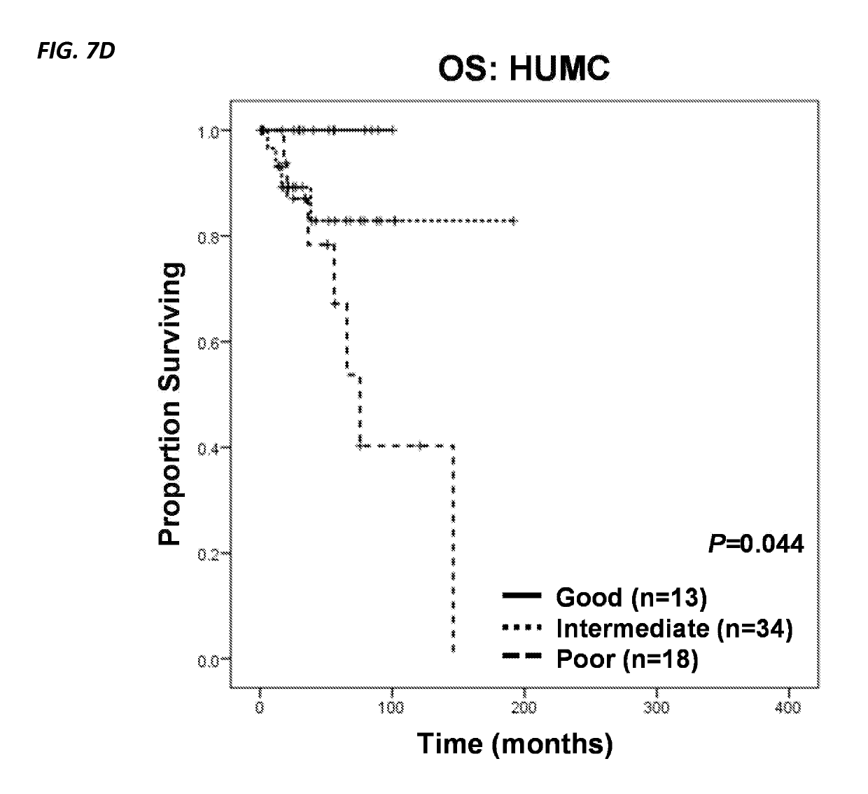

Importantly, highly significant separation was observed between the three groups when tested for association with TTFT and OS (p<0.001, FIGS. 2A and B, respectively). FIG. 2 shows Kaplan Meier plots for combined DS1 and DS2 treatment-naive specimens (n=228) classified into one of three groups (poor, intermediate, good) based on 20 CNAs in a hierarchical manner. Plots are shown for TTFT (FIG. 2A) and OS (FIG. 2B). The p-values provided are those obtained using the log rank test between good and intermediate groups and intermediate and poor groups, showing significant separation for OS and between poor and intermediate-good for TTFT. Individual specimen classifications are listed in Supplemental Table I from Houldsworth et al., Leukemia & Lymphoma 55:920-928 (2014). Pairwise, all showed significant separation except between intermediate and good prognosis groups for the TTFT endpoint (FIG. 2A). Within the good and intermediate groups, IGHV mutation status permitted additional significant stratification of patients for both endpoints (FIGS. 6A, B, D, and E). Overall then, the presence/absence of 20 CNAs as assessed by aCGH permitted classification of all CLL specimens into one of three groups that significantly correlated with time to first treatment and outcome. Validation of the hierarchical classification was performed in two previously untreated CLL datasets from independent institutions (Table 8). The first (DFCI) comprised 124 specimens submitted to high resolution single nucleotide polymorphism (SNP) array analysis and the second (HUMC), 65 specimens submitted to targeted aCGH. All specimens were classified into one of three prognostic groups according to the presence/absence of the 20 aberrations and separately tested for association with TTFT and OS (FIG. 7). In the DFCI dataset, association of aCGH outcome group with TTFT was validated (p<0.001), but not for OS (p=0.522), most likely explained by the low number of deaths in this dataset. Significant association of aCGH outcome group with OS was observed for the HUMC dataset (p=0.044), but not TTFT, where the median TTFT was only 17.2 months, being much shorter than expected for an average CLL dataset.

TABLE 8
Features of Datasets Used to Evaluate Clinical Associations

| Feature | | DS1 + DS2 | DFCI | HUMC |
|---|---|---|---|---|
| Untreated Specimens | | 228 | 124 | 65 |
| Median Diagnosis to Sampling | | 23.9 | 34.7 | Not |
| (Months) | | | | Available |
| Median Survivor Follow-Up | | 78 | 107 | 33.9 |
| (Months) | | | | |
| Treatment Events (n) | | 68 | 53 | 31 |
| Median TTFT (Months) | | 42.1 | 53.8 | 17.2 |
| Deaths (n) | | 40 | 11 | 11 |
| Median OS (months) | | 75.6 | 105.5 | 34.6 |
| aCGH Outcome | Poor | 20.60% | 11.30% | 27.70% |
| Group | Intermediate | 46.90% | 37.90% | 52.30% |
| | Good | 32.50% | 50.80% | 20.00% |

Other studies have reported an association between increased genomic complexity and adverse outcome in CLL, and a similar association was observed for the present CLL datasets (p<0.001), when those exhibiting two or more of the above 20 CNAs (72 of 228 cases) were considered complex. As expected a higher frequency of genomic complexity was noted within specimens from treated patients (29 of 60).

### Impact of Other Known Genome-Based Markers on Outcome in CLL

In order to examine the impact of TP53, NOTCH1, and SF3B1 mutations on the aCGH classification scheme, genomic DNA from specimens from untreated patients in DS1 was analyzed for TP53 (exons 5-9), NOTCH1 (exon 34), and SF3B1 (exons 14-16) mutations. TP53 mutations were identified in eight specimens (9.9%) (FIG. 1), including two with 17p13 loss and another two with another poor aCGH marker. Three were observed in specimens displaying 13q14 deletions and a mutated IGHV clonal rearrangement, in which cases, reportedly, survival is not negatively impacted by the mutation (Gonzalez et al., J. Clin. Oncol. 29:2223-2229 (2011)). In the present disclosure, collectively the presence of a TP53 mutation did not correlate with shorter TTFT or OS, but significantly correlated with adverse outcome and shorter TTFT when the three specimens with mutated IGHV were not considered positive (Table 6). Four specimens contained NOTCH1 mutations that correlated with shorter TTFT and OS (FIG. 1, Table 6) (Balatti et al., Blood 119:329-331 (2012); Puente et al., Nature 475:101-105 (2011); Rossi et al., Blood 119:521-529 (2012)). All four were in unmutated IGHV specimens, of which one carried a poor aCGH marker other than 17p or 11q loss, and one had gain of chromosome 12. As expected, the most common NOTCH1 mutation observed was ΔCT7544-7545, in three of the four (Balatti et al., Blood 119:329-331 (2012); Puente et al., Nature 475:101-105 (2011); Rossi et al., Blood 119:521-529 (2012)). SF3B1 missense mutations were detected in three specimens, all occurring in unmutated IGHV specimens and at previously reported hotspots, but correlated only with shorter TTFT (p=0.004). The presence of either clinically relevant TP53, NOTCH1, or SF3B1 mutation was in mutually exclusive specimens, and highly correlated with the presence of an unfavorable aCGH marker (66.7%), but less so when only considering aberrations associated with poor prognosis and routinely detected by FISH (del(17p), del(11q)) (33.3%).

### 13q14 Deletion Type and Association with Outcome

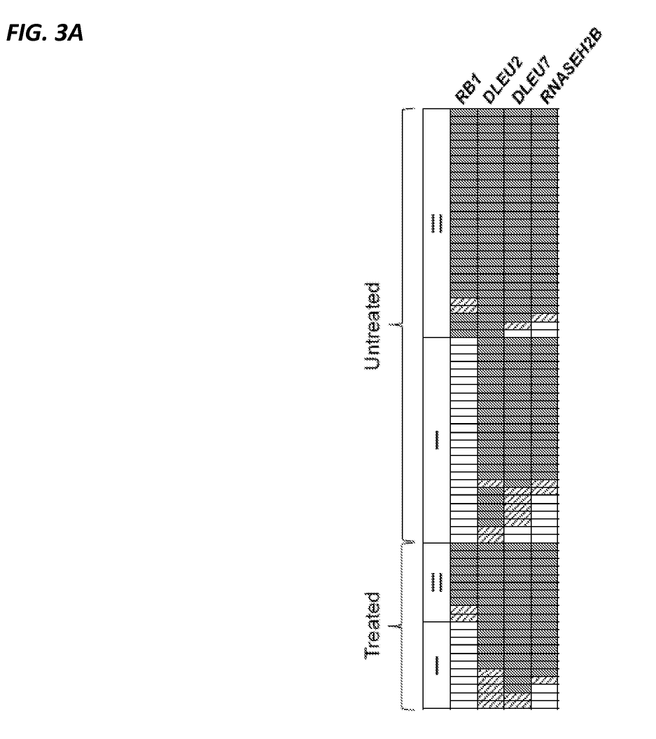

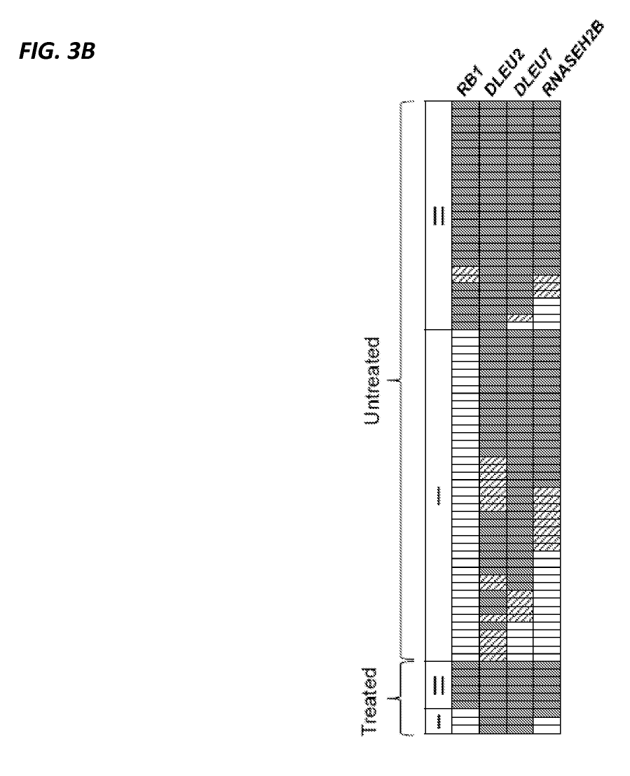

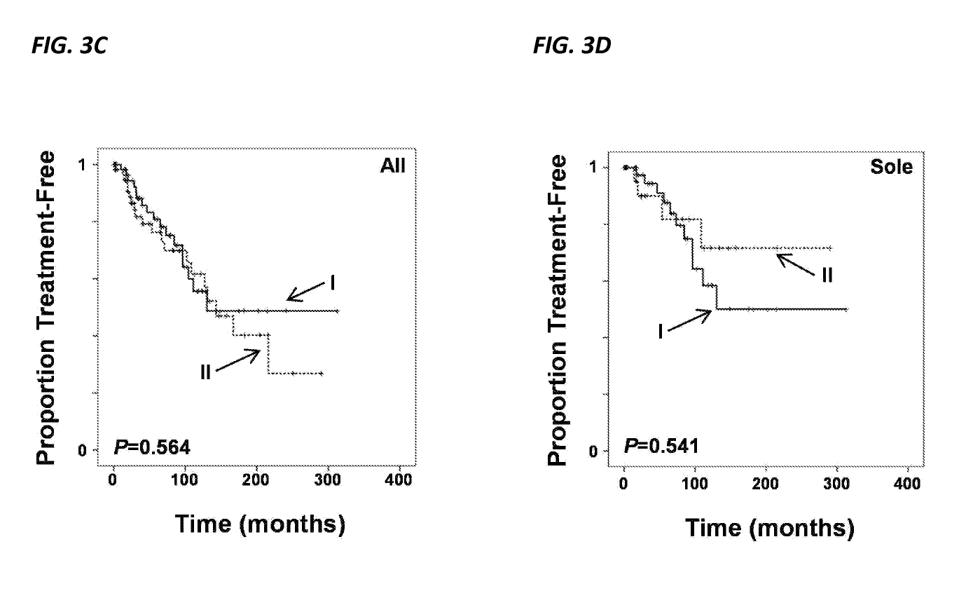

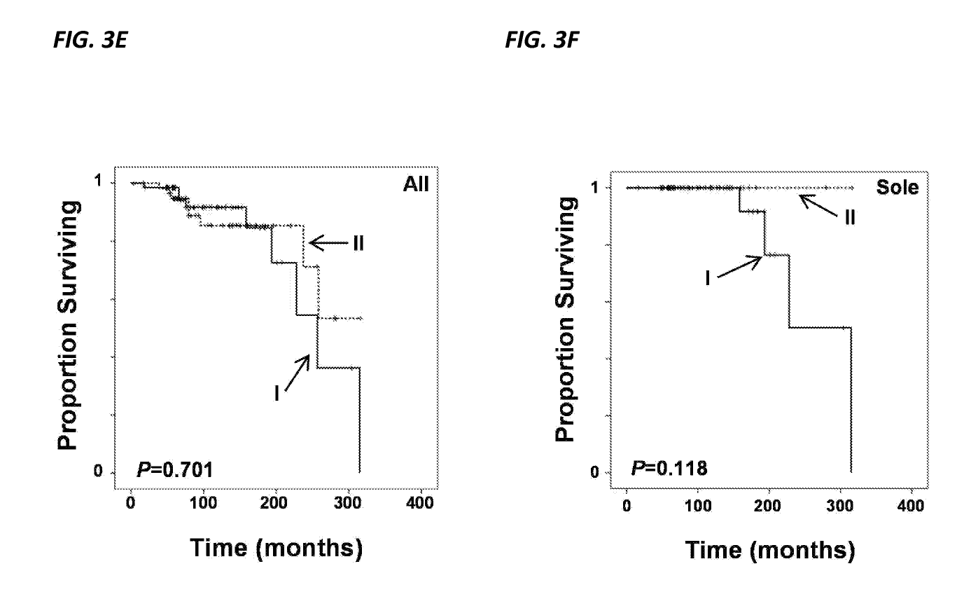

GISTIC analysis revealed that the peak region of the 13q14 deletion overlapped with the DLEU2 locus and promoter region. In order to define the 13q14 deletion in the present datasets, samples were recorded according to the CBS segmented, median-normalized log ratios at the RB1, DLEU2, DLEU7, and RNASEH2B loci (FIG. 3A-B, Table 9). In FIG. 3, specimens in DS1 (FIG. 3A) and DS2 (FIG. 3B) with 13q14 deletions were classified as type I or type II based on exclusion or inclusion of or part thereof RB1. The clinical relevance of type I or II deletion was assessed in treatment-naïve specimens of DS1 and DS2 combined (FIGS. 3C and D) in all with 13q14 deletions (All) and in those with 13q14 as a sole abnormality (Sole). Individual specimen classifications are listed in Table 9. Specimens are listed showing all (filled) or partial (hatched) loss of each of the four genic loci: RB1 (chr13:47.779-47.955 Mbp), DLEU2 (chr13:49,452-49,599 Mbp), DLEU7 (chr13:50,187-50,313 Mbp), and RNASEH2B (chr13: 50,397-50,439 Mbp) loci.

TABLE 9
Loss of Genic Loci at 13q14

| | | RB1 | DLEU2 | DLEU7 | RNASEH2B |
|---|---|---|---|---|---|
| | 13q Deletion | chr13: | chr13: | chr13: | chr13: |
| Sample ID | Type | 47.779-47.955* | 49.452-49.599 | 50.187-50.313 | 50.397-50.439 |
| DS1-1049 | I | 0 | 1 | 1 | 1 |
| DS1-1058 | II | 1 | 1 | 1 | 1 |
| DS1-1099 | I | 0 | 1 | Partial | 0 |
| DS1-1140 | II | Partial | 1 | 1 | 1 |
| DS1-1150 | II | 1 | 1 | 1 | 1 |
| DS1-1158 | II | 1 | 1 | 1 | 1 |
| DS1-1168 | II | 1 | 1 | 1 | 1 |
| DS1-1222 | II | 1 | 1 | 1 | 1 |
| DS1-1240 | II | 1 | 1 | 1 | 1 |
| DS1-1241 | II | 1 | 1 | 1 | 1 |
| DS1-1271 | I | 0 | 1 | 1 | 1 |
| DS1-1294 | II | 1 | 1 | 1 | 1 |
| DS1-1299 | II | Partial | 1 | 1 | 1 |
| DS1-1319 | II | 1 | 1 | 1 | 1 |
| DS1-1329 | II | 1 | 1 | 1 | 1 |
| DS1-1330 | I | 0 | 1 | 1 | 1 |
| DS1-1333 | II | 1 | 1 | 1 | 1 |
| DS1-1344 | I | 0 | 1 | 1 | 1 |
| DS1-1358 | II | 1 | 1 | 1 | 1 |
| DS1-1388 | I | 0 | 1 | 1 | 1 |
| DS1-156 | II | 1 | 1 | 1 | 1 |
| DS1-169 | I | 0 | 1 | 1 | 1 |
| DS1-171 | I | 0 | 1 | 1 | Partial |
| DS1-215 | II | 1 | 1 | 1 | 1 |
| DS1-257 | I | 0 | 1 | 1 | 1 |
| DS1-263 | II | 1 | 1 | 1 | 1 |
| DS1-271 | II | Partial | 1 | 1 | 1 |
| DS1-275 | I | 0 | Partial | 0 | 0 |
| DS1-276 | I | 0 | 1 | 1 | 1 |
| DS1-280 | II | 1 | 1 | 1 | 1 |
| DS1-316 | II | 1 | 1 | 1 | 1 |
| DS1-336 | I | 0 | 1 | 1 | 1 |
| DS1-342 | II | 1 | 1 | 1 | 1 |
| DS1-344 | I | 0 | Partial | 1 | 0 |
| DS1-348 | I | 0 | Partial | 1 | Partial |
| DS1-373 | I | 0 | 1 | 1 | 1 |
| DS1-377 | II | Partial | 1 | 1 | 1 |
| DS1-403 | I | 0 | 1 | Partial | Partial |
| DS1-430 | I | 0 | 1 | 1 | 1 |
| DS1-435 | II | 1 | 1 | 0 | 0 |
| DS1-453 | I | 0 | Partial | 1 | 1 |
| DS1-487 | II | 1 | 1 | 1 | 1 |
| DS1-499 | I | 0 | 1 | 1 | 1 |
| DS1-505 | II | 1 | 1 | 1 | 1 |
| DS1-574 | I | 0 | 1 | Partial | 0 |
| DS1-606 | II | 1 | 1 | Partial | 0 |
| DS1-625 | II | 1 | 1 | 1 | 1 |
| DS1-626 | II | 1 | 1 | 1 | 1 |
| DS1-654 | I | 0 | 1 | 1 | 1 |
| DS1-665 | I | 0 | 1 | Partial | 0 |
| DS1-667 | I | 0 | 1 | 1 | 1 |
| DS1-733 | I | 0 | 1 | 1 | 1 |
| DS1-738 | II | 1 | 1 | 1 | 1 |
| DS1-746 | I | 0 | 1 | 1 | 1 |
| DS1-766 | II | 1 | 1 | 1 | 1 |
| DS1-774 | I | 0 | Partial | Partial | 0 |
| DS1-794 | I | 0 | 1 | 1 | 1 |
| DS1-809 | II | 1 | 1 | 1 | 1 |
| DS1-815 | I | 0 | 1 | 1 | 1 |
| DS1-822 | I | 0 | 1 | 1 | 1 |
| DS1-834 | II | 1 | 1 | 1 | 1 |
| DS1-849 | I | 0 | 1 | 1 | 1 |
| DS1-854 | II | 1 | 1 | 1 | 1 |
| DS1-862 | I | 0 | Partial | 0 | 0 |
| DS1-863 | II | 1 | 1 | 1 | 1 |
| DS1-868 | I | 0 | 1 | 1 | 1 |
| DS1-870 | II | 1 | 1 | 1 | 1 |
| DS1-877 | I | 0 | 1 | Partial | 0 |
| DS1-880 | II | 1 | 1 | 1 | Partial |
| DS1-897 | II | 1 | 1 | 1 | 1 |
| DS1-909 | I | 0 | Partial | Partial | 0 |
| DS1-910 | II | 1 | 1 | 1 | 1 |
| DS1-923 | I | 0 | 1 | 1 | 1 |
| DS1-93 | I | 0 | 1 | 1 | 1 |
| DS1-942 | II | 1 | 1 | 1 | 1 |
| DS1-950 | I | 0 | 1 | 1 | 1 |
| DS2-102 | II | 1 | 1 | Partial | 0 |
| DS2-103 | I | 0 | 1 | 1 | 0 |
| DS2-105 | I | 0 | Partial | 1 | 1 |
| DS2-106 | I | 0 | Partial | Partial | 0 |
| DS2-107 | I | 0 | Partial | 0 | 0 |
| DS2-108 | I | 0 | 1 | 1 | 1 |
| DS2-111 | I | 0 | 1 | 1 | 1 |
| DS2-112 | II | Partial | 1 | 1 | Partial |
| DS2-113 | II | 1 | 1 | 1 | 1 |
| DS2-114 | II | 1 | 1 | 1 | Partial |
| DS2-115 | II | 1 | 1 | 1 | 1 |
| DS2-132 | I | 0 | 1 | 1 | 1 |
| DS2-135 | I | 0 | 1 | 1 | 1 |
| DS2-14 | II | 1 | 1 | 1 | 0 |
| DS2-142 | II | 1 | 1 | 1 | 1 |
| DS2-143 | I | 0 | 1 | 1 | 1 |
| DS2-15 | I | 0 | 1 | 1 | 0 |
| DS2-150 | I | 0 | 1 | 1 | 0 |
| DS2-153 | II | 1 | 1 | 1 | 1 |
| DS2-161 | I | 0 | 1 | 1 | 1 |
| DS2-162 | II | 1 | 1 | 1 | 1 |
| DS2-167 | II | 1 | 1 | 1 | 1 |
| DS2-168 | I | 0 | Partial | 1 | 1 |
| DS2-17 | II | 1 | 1 | 1 | 1 |
| DS2-171 | I | 0 | 1 | 1 | Partial |
| DS2-173 | II | 1 | 1 | 1 | 1 |
| DS2-174 | II | 1 | 1 | 1 | 1 |
| DS2-175 | II | 1 | 1 | 1 | 1 |
| DS2-178 | II | 1 | 1 | 1 | 1 |
| DS2-181 | I | 0 | 1 | 1 | 1 |
| DS2-19 | II | 1 | 1 | 1 | 1 |
| DS2-194 | II | 1 | 1 | 1 | 1 |
| DS2-204 | I | 0 | Partial | 0 | 0 |
| DS2-209 | II | 1 | 1 | 1 | 1 |
| DS2-21 | I | 0 | Partial | 0 | 0 |
| DS2-210 | I | 0 | 1 | Partial | 0 |
| DS2-216 | I | 0 | 1 | 1 | Partial |
| DS2-217 | I | 0 | 1 | 1 | 0 |
| DS2-22 | I | 0 | Partial | 0 | 0 |
| DS2-225 | I | 0 | 1 | 1 | 1 |
| DS2-23 | II | 1 | 1 | 1 | Partial |
| DS2-233 | I | 0 | 1 | Partial | 0 |
| DS2-235 | II | 1 | 1 | 1 | 1 |
| DS2-236 | I | 0 | 1 | 1 | Partial |
| DS2-237 | I | 0 | Partial | 1 | Partial |
| DS2-239 | I | 0 | 1 | 0 | 0 |
| DS2-240 | I | 0 | Partial | 1 | 1 |
| DS2-241 | I | 0 | 1 | 1 | Partial |
| DS2-243 | II | 1 | 1 | 1 | 1 |
| DS2-244 | I | 0 | 1 | 1 | 1 |
| DS2-250 | I | 0 | 1 | 1 | 1 |
| DS2-254 | II | 1 | 1 | 1 | 1 |
| DS2-256 | II | 1 | 1 | 1 | 1 |
| DS2-258 | I | 0 | Partial | 1 | Partial |
| DS2-259 | I | 0 | 1 | 1 | Partial |
| DS2-26 | II | 1 | 1 | 1 | 1 |
| DS2-28 | I | 0 | 1 | 1 | 1 |
| DS2-29 | I | 0 | 1 | 1 | 1 |
| DS2-32 | II | 1 | 1 | 1 | 1 |
| DS2-33 | I | 0 | Partial | 1 | 0 |
| DS2-41 | II | 1 | 1 | 1 | 1 |
| DS2-48 | II | 1 | 1 | 1 | 1 |
| DS2-49 | II | 1 | 1 | 1 | 0 |
| DS2-5 | II | Partial | 1 | 1 | 1 |
| DS2-51 | II | 1 | 1 | 1 | 1 |
| DS2-55 | II | 1 | 1 | 1 | 1 |
| DS2-56 | I | 0 | 1 | Partial | 0 |
| DS2-57 | I | 0 | 1 | 1 | 1 |
| DS2-61 | I | 0 | 1 | 1 | 1 |
| DS2-64 | I | 0 | 1 | 1 | 1 |
| DS2-68 | II | 1 | 1 | 1 | 1 |
| DS2-73 | I | 0 | Partial | 1 | Partial |
| DS2-75 | I | 0 | 1 | 1 | 1 |
| DS2-76 | I | 0 | 1 | 1 | 0 |
| DS2-79 | I | 0 | Partial | 1 | 1 |
| DS2-8 | II | 1 | 1 | 1 | 1 |
| DS2-83 | II | 1 | 1 | 0 | 0 |
| DS2-87 | I | 0 | 1 | 1 | 1 |
| DS2-94 | II | 1 | 1 | 1 | 1 |

*Mbp

The entire DLEU2 genic region was deleted in most cases, but partial losses were detected in 22 specimens, all of which included the MIR-15A/16.1 locus with the exception of four, for which the telomeric portion of DLEU2 was deleted along with promoter sequences. The smallest detected partial deletion of DLEU2 was in case DS2-204 of 366 kbp (chr13:49,464,630-49,830,378). In treatment-naive specimens with 13q14 deletions, 47.3% of DS1 were Type I 13q14 deletions, and 59.2% of DS2. When combined and tested for association with clinical endpoints, no significant difference in OS or TTFT was found between deletion type (FIG. 3C-D), nor when present as a sole abnormality (FIG. 3C-D). FIG. 3C shows the TTFT clinical endpoint, and FIG. 3D shows the OS clinical endpoint. The clinical relevance of the telomeric breakpoint was also examined, where the majority of cases (94.2%) exhibiting loss of DLEU2, also displayed loss or partial loss of DLEU7 (FIG. 3A-B). Fewer exhibited concurrent deletion of RNASEH2B (80.1%). No significant association with TTFT or OS was found with the extended deletion including one or both telomeric loci, with the exception of longer OS when the deletion extended only to include DLEU7 (p=0.036).

### DISCUSSION

In the present disclosure, a defined panel of genomic CNAs have been identified by aCGH that collectively allow hierarchical classification of all specimens from treatment-naïve CLL patients for risk stratification into one of three groups with poor, intermediate, or good prognosis. Nine were biomarkers of adverse outcome (gain: 2p, 3q, 8q, 17q; loss: 7q, 8p, 11q, 17p, 18p) and ten others (gain: 1p, 7p, 12, 18p, 18q, 19; loss: 4p, 5p, 6q, 7p) were used to define loss of 13q14 as a sole abnormality. Prior aCGH studies have reported associations of CNAs with outcome, but none until now have integrated the findings for definitive classification of specimens for clinical utility. Importantly, mutations in the TP53, NOTCH1, and SF3B1 genes were found to be highly correlated with the presence of a poor aCGH CNA—higher than would have been found based solely on the loss of 17p or 11q, as routinely assessed by FISH. Collectively, these findings demonstrate the utility of aCGH to detect genomic imbalance in CLL with prognostic significance in a clinical diagnostic setting.

Nine aCGH aberrations were found to be associated with adverse outcome and shorter time to first treatment, including the well-described losses of 17p and 11q. Much less is known for the low frequency gains of 2p, 3q, 8q, and 17q, and loss of 7q, 8p, and 18p. These aberrations have been reported in other CLL datasets, often at higher frequencies in progressed and relapsed patients, and sometimes with clinical relevance (Grubor et al., Blood 113:1294-1303 (2009); Rinaldi et al., Br. J. Haematol. 154:590-599 (2011); Brown et al., Clin. Cancer Res. 18:3791-3802 (2012); Gunn et al., J. Mol. Diagn. 10:442-451 (2008); Ouillette et al., Blood 118:3051-3061 (2011); Pfeifer et al., Blood 109:1202-1210 (2007); Gunnarsson et al., Haematologica 96:1161-1169 (2011); Kujawski et al., Blood 112:1993-2003 (2008); Schultz et al., Mol. Cytogenet. 4:4 (2011); Fabris et al., Am. J. Hematol. 88:24-31 (2013); Woyach et al., Br. J. Haematol. 148:754-759 (2010); Rudenko et al., Leuk. Lymphoma 49:1879-1886 (2008)). The presence of several of the poor aCGH aberrations were found to be correlated, consistent with increased genomic complexity observed in CLL specimens portending adverse outcome and less durable responses, which was also confirmed in the present disclosure (Ouillette et al., Blood 118:3051-3061 (2011); Pfeifer et al., Blood 109:1202-1210 (2007); Kujawski et al., Blood 112:1993-2003 (2008); Kay et al., Cancer Genet. Cytogenet. 203:161-168 (2010)). Other studies have implicated NCOA2, ROCK2, REL, MYCN (2p), PIK3CA (3q), CAV1 (7q), TNFSF10A/B (8p), MYC (8q), ATM (11q), and TP53 (17p) as potential target genes for the respective regions based on matched expression and mutation analyses, but their true roles in CLL remain unclear (Rinaldi et al., Br. J. Haematol. 154:590-599 (2011); Brown et al., Clin. Cancer Res. 18:3791-3802 (2012); Fabris et al., Am. J. Hematol. 88:24-31 (2013); Woyach et al., Br. J. Haematol. 148:754-759 (2010); Stankovic & Skowronska, Leuk. Lymphoma (2013); Forconi et al., Br. J. Haematol. 143:532-536 (2008)). Deletion of 6q was identified in the present disclosure as a recurrent aberration, but did not significantly correlate with disease progression or overall outcome. The clinical relevance of this CNA has been inconsistent across studies, perhaps explained by a minimally deleted region centered at 6q21 that does not include the MYB locus, commonly used in FISH for the detection of this abnormality (Gunn et al., J. Mol. Diagn. 10:442-451 (2008); Cuneo et al., Leukemia 18:476-483 (2004)).

Since the first report of the prognostic relevance of different centromeric breakpoints of deletions at 13q14, there have been other studies with mixed support for the relevance of the two types (Ouillette et al., Clin. Cancer Res. 17:6778-6790 (2011); Dal Bo et al., Genes Chromosomes Cancer 50:633-643 (2011); Mian et al., Hematol. Oncol. 30:46-49 (2012); Mosca et al., Clin. Cancer Res. 16:5641-5653 (2010); Parker et al., Leukemia 25:489-497 (2011)). In the present disclosure, an association of type with outcome was not confirmed in those having 13q14 deletion, or those detected as a sole abnormality. The significance of the clinical relevance of the telomeric breakpoints is much less known, but murine studies have revealed a role for the DLEU7/RNASE7H loci in progression of MBL to CLL, and a germline deletion of this locus has been reported in a family with CLL (Rossi et al., Blood 118:1877-1884 (2011); Brown et al., Leukemia 26:1710-1713 (2012)). Most specimens exhibited loss of DLEU7, which is perhaps not surprising given that all patients were diagnosed with CLL. Thus, despite the ability of aCGH to accurately define different size deletions at 13q14, the clinical relevance remains unclear.

Currently in CLL, determination of IGHV mutation status and detection of genomic imbalance by FISH are recommended as part of risk stratification (NCCN, Non-Hodgkin's Lymphomas, NCCN Clinical Practice Guidelines in Oncology 2011, Version 4.2011). Unfortunately, of the four loci evaluated by FISH, no additional outcome stratification is afforded within those CLL patients who do not bear 17p or 11q loss (up to 85% of unselected patients). Indeed, no difference in OS has been reported for patients with del(13q) as a sole abnormality (based on the four loci) versus those with trisomy 12 or no aberrations (Van Dyke et al., Br. J. Haematol. 148:544-550 (2010)). Importantly, the present disclosure not only identified additional patients with adverse outcome and shorter time to first treatment, other than those with del(17p) or del(11q), but it also allowed significant stratification of all remaining specimens into either a good or an intermediate prognosis group. Unlike FISH-based prognosis (Dohner et al., N. Engl. J. Med. 343:1910-1916 (2000)), the presently disclosed aCGH-based hierarchical scheme allows stratification of all specimens.

Deep sequencing studies have identified several somatic genic mutations including NOTCH1, SF3B1, and BIRC3 that associate with poor prognosis (Balatti et al., Blood 119:329-331 (2012); Fabbri et al., J. Exp. Med. 208:1389-1401 (2011); Puente et al., Nature 475:101-105 (2011); Rossi et al., Blood 119:521-529 (2012); Rudenko et al., Leuk. Lymphoma 49:1879-1886 (2008)). Disruption of BIRC3, however, is mostly evidenced as bi-allelic deletion or mono-allelic deletion with mutational inactivation of the remaining allele (Rossi et al., Blood 119:2854-2862 (2012)). In the present disclosure, deletion of the BIRC3 locus without concurrent deletion of ATM was rare, and observed in one treated (DS2-235) and one untreated specimen (DS1-1344), which also exhibited gain of 2p. All those with NOTCH1 mutations in the present disclosure also had unmutated IGHV, consistent with other studies, but only one also exhibited trisomy 12 as a sole abnormality (Balatti et al., Blood 119:329-331 (2012)). Overrepresentation of NOTCH1 mutations has been reported in cases with trisomy 12, and it is possible that differences in specimen selection could account for the differences in observed frequency (Balatti et al., Blood 119:329-331 (2012)). SF3B1 mutations occurred at a frequency comparable with other unselected untreated CLL specimen datasets and at similarly reported hotspots (Rossi et al., Blood 118:6904-6908 (2011); Wang et al., N. Engl. J. Med. 365:2497-2506 (2011)). In the present disclosure, NOTCH1, TP53, and SF3B1 mutations were found to occur largely in non-overlapping specimens that bore poor risk aCGH CNAs, often not 11q or 17p loss. This novel finding suggests that in a clinical diagnostic setting, aCGH could be utilized as a stand-alone assay to identify most CLL patients with an adverse outcome. This represents more than those currently identified by FISH alone and also identifies a large proportion of those bearing somatic mutations known to impact survival, thereby reducing the need to perform labor-intensive and costly sequence analysis for each gene for every specimen. While aCGH exhibits reduced sensitivity compared with FISH, it does, by virtue of the ability to obtain genomic gain/loss information at more loci, provide further risk stratification of patients not bearing any poor aCGH marker, and also allows an evaluation of genomic complexity, as supported by the present disclosure, that correlates with adverse outcome and is mostly observed in specimens bearing poor aCGH markers. In summary, while the CLL genome is on the whole relatively quiet, genomic imbalance as assessed by aCGH in a clinical diagnostic setting can serve as a powerful prognostic tool for risk stratification in CLL patients.

TABLE 10
Summary of SEQ ID NOS

| SEQ ID NO | AA/DNA | Description |
|---|---|---|
| 1 | DNA | Forward PCR primer (exons 5-6) |
| 2 | DNA | Reverse PCR primer (exons 5-6) |
| 3 | DNA | Forward PCR primer (exons 7-9) |
| 4 | DNA | Reverse PCR primer (exons 7-9) |
| 5 | DNA | Forward sequencing primer 2 (exons 7-9) |
| 6 | DNA | Reverse sequencing primer 2 (exons 7-9) |
| 7 | DNA | Forward sequencing primer |
| 8 | DNA | Reverse sequencing primer |
| 9 | DNA | Forward PCR primer (exon 14) |
| 10 | DNA | Reverse PCR primer (exon 14) |
| 11 | DNA | Forward PCR primer (exons 15-16) |
| 12 | DNA | Reverse PCR primer (exons 15-16) |
| 13 | DNA | Reverse sequencing primer, SR (exon 14) |

All publications and patent applications mentioned in the specification are indicative of the level of those skilled in the art to which this invention pertains. All publications and patent applications are herein incorporated by reference in their entirety for all purposes to the same extent as if each individual publication or patent application was specifically and individually indicated to be incorporated by reference.

Throughout the specification the terms “comprising” and “including” or variations such as “comprises” or “includes,” will be understood to imply the inclusion of a stated element, integer or step, or group of elements, integers or steps, but not the exclusion of any other element, integer or step, or group of elements, integers or steps.

As used herein, the term “about,” when referring to a value, is meant to encompass variations of, in some embodiments +/−50%, in some embodiments +/−20%, in some embodiments +/−10%, in some embodiments +/−5%, in some embodiments +/−1%, in some embodiments +/−0.5%, and in some embodiments +/−0.1% from the specified amount, as such variations are appropriate to perform the disclosed methods or employ the disclosed compositions.

Where a range of numerical values is recited herein, unless otherwise stated, the range is intended to include the endpoints thereof, all possible subranges within the range, and all integers and fractions within the range. It is not intended that the scope of the presently disclosed subject matter be limited to the specific values recited when defining a range.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for risk stratification of a human chronic lymphocytic leukemia (CLL) patient, the method comprising:
(a) providing a microarray comprising a substrate with a plurality of distinct genomic regions, wherein each of the distinct genomic regions is individually capable of hybridizing to sample genetic material from the CLL patient, wherein the distinct genomic regions comprise:
(i) genomic regions comprising regions defined by the coordinates specified as peak limits for each of the genomic regions identified in Table 5;
(ii) a genomic region comprising the region between coordinates 122,471,896-124,803,693 on chromosome 7; and
(iii) a genomic region comprising the region between coordinates 5,460,990-8,079,142 on chromosome 5;
(b) providing the sample genetic material and labeled reference genetic material, wherein the sample genetic material is labeled sample genetic material;
(c) hybridizing the labeled sample genetic material and the labeled reference genetic material with the distinct genomic regions arrayed on the substrate;
(d) analyzing the hybridization pattern of the labeled sample genetic material to the distinct genomic regions relative to the hybridization pattern of the reference genetic material to the distinct genomic regions to detect the presence of copy number alterations in the sample genetic material; and
(e) stratifying the CLL patient into one of the following risk groups:
(i) poor prognosis: CLL patients whose sample genetic material comprises at least one of gain of 2p, gain of 3q, gain of 8q, gain of 17q, loss of 7q, loss of 8p, loss of 11q, loss of 17p, and loss of 18p;
(ii) good prognosis: CLL patients whose sample genetic material comprises loss of 13q14 without any of the copy number alterations listed in step (e)(i) and without any of gain of 1p, gain of 7p, gain of 12, gain of 18p, gain of 18q, gain of 19, loss of 4p, loss of 5p, loss of 6q, and loss of 7p; and
(iii) intermediate prognosis: all other CLL patients.

2. The method of claim 1, wherein the distinct genomic regions comprise genomic regions comprising the regions listed in Table 2.

3. The method of claim 1, wherein the sample genetic material and the reference genetic material are hybridized with the distinct genomic regions arrayed on the substrate at the same time.

4. The method of claim 3, wherein the labeled sample genetic material comprises a first label and the labeled reference genetic material comprises a second label, wherein the first label and the second label are non-identical and can be detected simultaneously when hybridized to at least one of the distinct genomic regions arrayed on the substrate.

5. The method of claim 1, wherein the distinct genomic regions are between about 0.3 Mbp to about 21.3 Mbp in size and are represented on the microarray at a resolution with an average density of about 35 kbp.

6. The method of claim 1, wherein the substrate further comprises a backbone probe set arrayed thereon that covers the entire chromosomal complement, and wherein the hybridizing step further comprises hybridizing the sample genetic material and the reference genetic material with the backbone probe set arrayed on the substrate.

7. The method of claim 6, wherein the backbone probe set covers the entire chromosomal complement at a resolution with an average density of about 1 Mbp.

8. The method of claim 6, wherein the backbone probe set excludes genomic regions of known copy number variation.

9. The method of claim 1, wherein the CLL patient is a treatment-naïve patient.

10. The method of claim 1, wherein the poor prognosis is shorter predicted time to first treatment and/or shorter predicted overall survival and the good prognosis is longer predicted time to first treatment and/or longer predicted overall survival.

11. The method of claim 1, further comprising further stratifying the CLL patient based on IGHV mutation status, wherein mutated IGHV predicts a better prognosis and unmutated IGHV predicts a worse prognosis for CLL patients in the good prognosis and intermediate prognosis groups.

12. The method of claim 11, wherein the worse prognosis is shorter predicted time to first treatment and/or shorter predicted overall survival and the better prognosis is longer predicted time to first treatment and/or longer predicted overall survival.

13. A system for risk stratification of a human CLL patient, the system comprising:
(a) a microarray comprising a substrate with a plurality of distinct genomic regions, wherein each of the distinct genomic regions is individually capable of hybridizing to sample genetic material from the CLL patient, wherein the distinct genomic regions comprise:
(i) genomic regions comprising regions defined by the coordinates specified as peak limits for each of the genomic regions identified in Table 5;
(ii) a genomic region comprising the region between coordinates 122,471,896-124,803,693 on chromosome 7; and
(iii) a genomic region comprising the region between coordinates 5,460,990-8,079,142 on chromosome 5; and
(b) a decision tree comprising steps for stratification of the CLL patient into one of the following groups:
(i) poor prognosis: CLL patients whose sample genetic material comprises at least one of gain of 2p, gain of 3q, gain of 8q, gain of 17q, loss of 7q, loss of 8p, loss of 11q, loss of 17p, and loss of 18p;
(ii) good prognosis: CLL patients whose sample genetic material comprises loss of 13q14 without any of the copy number alterations listed in step (a) and without any of gain of 1p, gain of 7p, gain of 12, gain of 18p, gain of 18q, gain of 19, loss of 4p, loss of 5p, loss of 6q, and loss of 7p; and
(iii) intermediate prognosis: all other CLL patients.

14. The system of claim 13, wherein the distinct genomic regions comprise genomic regions comprising the regions listed in Table 2.

15. The system of claim 13, wherein the distinct genomic regions are between about 0.3 Mbp to about 21.3 Mbp in size and are represented on the microarray at a resolution with an average density of about 35 kbp.

16. The system of claim 13, wherein the substrate further comprises a backbone probe set arrayed thereon that covers the entire chromosomal complement.

17. The system of claim 16, wherein the backbone probe set covers the entire chromosomal complement at a resolution with an average density of about 1 Mbp.

18. The system of claim 16, wherein the backbone probe set excludes genomic regions of known copy number variation.

19. The system of claim 13, wherein the CLL patient is a treatment-naïve patient.

20. The system of claim 13, wherein the prognosis is predicted time to first treatment and/or predicted overall survival.


SEQUENCE LISTING (13 sequences)

SEQ ID NO: 1 - DNA, 20 bp, Artificial Sequence
  Synthetic
gtttctttgctgccgtcttc

SEQ ID NO: 2 - DNA, 20 bp, Artificial Sequence
  Synthetic
ttgcacatctcatggggtta

SEQ ID NO: 3 - DNA, 20 bp, Artificial Sequence
  Synthetic
aaaaggcctcccctgcttgc

SEQ ID NO: 4 - DNA, 20 bp, Artificial Sequence
  Synthetic
tgtctttgaggcatcactgc

SEQ ID NO: 5 - DNA, 21 bp, Artificial Sequence
  Synthetic
gggagtagatggagcctggtt

SEQ ID NO: 6 - DNA, 25 bp, Artificial Sequence
  Synthetic
gtcccatttaaaaaaccaggctcca

SEQ ID NO: 7 - DNA, 20 bp, Artificial Sequence
  Synthetic
ggcatggtgccgaaccaata

SEQ ID NO: 8 - DNA, 20 bp, Artificial Sequence
  Synthetic
tacttgaaggcctccggaat

SEQ ID NO: 9 - DNA, 24 bp, Artificial Sequence
  Synthetic
tctgtttatggaattgattatgga

SEQ ID NO: 10 - DNA, 20 bp, Artificial Sequence
  Synthetic
actaaggaggctgagcagga

SEQ ID NO: 11 - DNA, 21 bp, Artificial Sequence
  Synthetic
tgcagtttggctgaatagttg

SEQ ID NO: 12 - DNA, 26 bp, Artificial Sequence
  Synthetic
caaatcaaacagtattcgtgtaacat

SEQ ID NO: 13 - DNA, 24 bp, Artificial Sequence
  Synthetic
caacttaccatgttcaatgatttc